(19) United States
(12) Patent Application Publication
(10) Pub. No.: US 2025/0235508 A1
(43) Pub. Date: Jul. 24, 2025

(54) MODIFIED THERAPEUTIC AGENTS AND COMPOSITIONS THEREOF

(71) Applicant: THE SCRIPPS RESEARCH INSTITUTE, La Jolla, CA (US)

(72) Inventors: Weijun SHEN, San Diego, CA (US); Pengyu YANG, San Diego, CA (US); Peter G. SCHULTZ, La Jolla, CA (US)

(21) Appl. No.: 18/962,779

(22) Filed: Nov. 27, 2024

Related U.S. Application Data
(63) Continuation of application No. 17/485,171, filed on Sep. 24, 2021, now abandoned.
Application No. 17/485,171 is a continuation of application No. 15/735,898, filed on Dec. 12, 2017, now abandoned.
(60) Provisional application No. 62/180,856, filed on Jun. 17, 2015.

(51) Int. Cl. A61K 38/22; A61K 9/00; A61K 31/20; A61K 31/23; A61K 38/13; A61K 38/26; A61K 38/28; A61K 45/06; A61K 47/54; A61P 1/04; A61P 3/04; A61P 3/06; A61P 3/10; C07K 14/575; C07K 14/605

(52) U.S. Cl. CPC A61K 38/2264; A61K 9/0021; A61K 31/20; A61K 31/23; A61K 38/13; A61K 38/26; A61K 38/28; A61K 45/06; A61K 47/542; A61K 47/554; A61P 1/04; A61P 3/04; A61P 3/06; A61P 3/10; C07K 14/575; C07K 14/57563; C07K 14/5759; C07K 14/605

(57) ABSTRACT

Methods and compositions are provided for extending the half-life of a therapeutic agent. A modified therapeutic agent (mTA) comprises a therapeutic agent, a staple, and a half-life extending molecule. The mTAs disclosed herein may be used to treat a disease or a condition in a subject in need thereof.

### RELATED APPLICATIONS

This application is a continuation of U.S. patent application Ser. No. 17/485,171 filed on Sep. 24, 2021, which is a continuation of U.S. patent application Ser. No. 15/735,898 filed on Dec. 12, 2017, now abandoned, which is a 371 of International Application No. PCT/US2016/37834 filed Jun. 16, 2016, which claims the benefit of U.S. Provisional Patent Application No. 62/180,856 filed on Jun. 17, 2015, each of which is incorporated herein by reference in its entirety.

### SEQUENCE LISTING

The instant application contains a Sequence Listing submitted in electronic format. The Sequence Listing is provided as a file entitled 41135_746_302_SL.xml, created on Nov. 27, 2024, which is 67,920 bytes in size. The information in the electronic format of the Sequence Listing is incorporated by reference in its entirety.

### BACKGROUND OF THE INVENTION

The development of therapeutic agents (e.g., biological drugs) is often hampered by short half-lives. The biological half-life or elimination half-life of a substance is the time it takes for a substance (for example a metabolite, drug, signaling molecule, radioactive nuclide, or other substance) to lose half of its pharmacologic, physiologic, or radiologic activity. As a result of the short half-life, patients are often administered higher dosages more frequently, which may lead to reduced compliance, higher costs and greater risks of side effects.

Extended-release products are designed to prolong the absorption of drugs with short half-lives, thereby allowing longer dosing intervals while minimizing fluctuations in serum drug levels. Current strategies used for extending half-lives are those that increase hydrodynamic volume (PEGylation) or those that use FcRn-mediated recycling (albumin fusions). Attachment of polypeptides or lipophilic constituents to drugs has also been used to extend the half-life of a biological agent (U.S. Pat. Nos. 6,268,343; 5,750,497; 8,129,343).

The present disclosure provides modified therapeutic agents (mTAs) for improving the biological, chemical, physiologic, pharmacologic, pharmacokinetic, and/or pharmacodynamic properties of a therapeutic agent.

### SUMMARY OF THE INVENTION

Disclosed herein are modified therapeutic agents (mTAs) comprising a therapeutic agent, a first staple, and a first half-life extending molecule, a first staple, wherein the therapeutic agent is a modified or unmodified therapeutic peptide that is covalently attached to the first staple via two amino acid residues on the modified or unmodified therapeutic peptide; each of the two amino acid residues has an amine-containing sidechain for attachment to the first staple through the formation of an amide; the first half-life extending molecule is covalently attached to the first staple; and the half-life of the mTA is longer than the half-life of the unmodified therapeutic peptide alone. The first half-life extending molecule may comprise a lipid, a polyglycol region, or a combination thereof. The first half-life extending molecule may comprise a lipid. The first half-life extending molecule may comprise a lipid and a polyglycol region. The first half-life extending molecule may comprise a polyglycol region. The lipid may be selected from a group consisting of sterols, sterol derivatives, bile acids, vitamin E derivatives, fatty di-acids, fatty acids, fatty amides, fatty amines, and fatty alcohols, and derivatives thereof. The polyglycol region may comprise one or more polyethylene glycol units, polypropylene glycol units, or polybutylene glycol units, or a combination thereof. The polyglycol region may be selected from

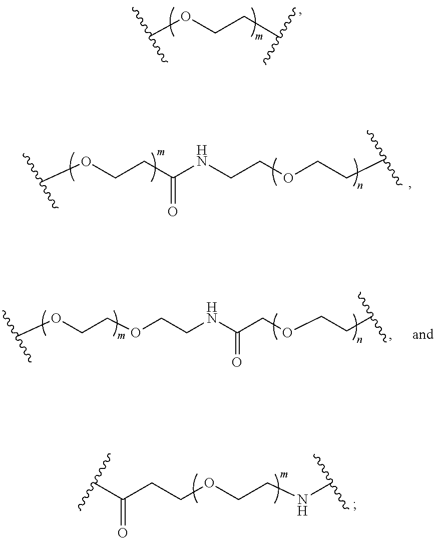

wherein
m and n are independently 1, 2, 3, 4, 5, 6, 7, 8, 9, 10, 11, 12, 13, 14, 15, 16, 17, 18, 19, or 20.

The modified therapeutic peptide may comprise one or more amino acid additions, deletions, or substitutions, or a combination thereof. The unmodified therapeutic peptide may be selected from GLP-1, glucagon, oxyntomodulin, exendin-4, GLP-2, GIP, GLP-1R/GCGR dual agonist, GLP-1R/GIPR dual agonist, and GLP-1R/GCGR/GIPR tri-agonist. The modified therapeutic peptide may be a derivative of a peptide selected from GLP-1, glucagon, oxyntomodulin, exendin-4, GLP-2, GIP, GLP-1R/GCGR dual agonist, GLP-1R/GIPR dual agonist, and GLP-1R/GCGR/GIPR tri-agonist; the derivative being a peptide comprising one or more amino acid additions, deletions, or substitutions, or a combination thereof. The modified therapeutic peptide may be a derivative of a peptide selected from GLP-1, glucagon, oxyntomodulin, exendin-4, GLP-2, and GIP; the derivative being a peptide comprising one or more amino acid additions, deletions, or substitutions, or a combination thereof. The modified or unmodified therapeutic peptide may comprise an amino acid sequence comprising at least a portion of a polypeptide sequence selected from a group consisting of SEQ ID NO: 1-30. The modified or unmodified therapeutic peptide may comprise an amino acid sequence comprising 10 or more amino acids based on or derived from a polypeptide sequence selected from a group consisting of SEQ ID NO: 1-30. The modified or unmodified therapeutic peptide may comprise an amino acid sequence that is at least about 50% homologous to an amino acid sequence selected from the group comprising SEQ ID NO: 1-30. The modified or unmodified therapeutic peptide may comprise an amino acid sequence that is at least 80% homologous to an amino acid sequence selected from the group comprising SEQ ID NO: 1-30. The modified or unmodified therapeutic peptide may comprise an amino acid sequence comprising at least a portion of a polypeptide sequence selected from a group consisting of SEQ ID NO: 1-6. The modified or unmodified therapeutic peptide may comprise an amino acid sequence comprising 10 or more amino acids based on or derived from a polypeptide sequence selected from a group consisting of SEQ ID NO: 1-6. The modified or unmodified therapeutic peptide may comprise an amino acid sequence that is at least about 50% homologous to an amino acid sequence selected from the group comprising SEQ ID NO: 1-6. The modified or unmodified therapeutic peptide may comprise an amino acid sequence that is at least 80% homologous to an amino acid sequence selected from the group comprising SEQ ID NO: 1-6. The modified or unmodified therapeutic peptide may comprise an amino acid sequence comprising at least a portion of a polypeptide sequence selected from a group consisting of SEQ ID NO: 7-30. The modified or unmodified therapeutic peptide may comprise an amino acid sequence comprising 10 or more amino acids based on or derived from a polypeptide sequence selected from a group consisting of SEQ ID NO: 7-30. The modified or unmodified therapeutic peptide may comprise an amino acid sequence that is at least about 50% homologous to an amino acid sequence selected from the group comprising SEQ ID NO: 7-30. The modified or unmodified therapeutic peptide may comprise an amino acid sequence that is at least 80% homologous to an amino acid sequence selected from the group comprising SEQ ID NO: 7-30. At least one of the two amino acid residues may be an amino acid addition or substitution on the modified therapeutic peptide. Each of the two amino acid residues may be independently selected from lysine, ornithine, diaminobutyric acid, diaminopropionic acid, and homolysine. Each of the two amino acid residues may be lysine. The mTA may further comprise a second staple. The mTA may further comprise a second half-life extending molecule. The mTA may further comprise a second staple and a second half-life extending molecule, wherein the second half-life molecule is covalently attached to the second staple. The half-life of the mTA may be 5-fold longer than the half-life of the unmodified therapeutic peptide alone.

Further disclosed herein are pharmaceutical compositions comprising an mTA disclosed herein and a pharmaceutically acceptable excipient.

Further disclosed herein are methods for treating a disease or condition in a subject in need thereof, the method comprising administering to the subject a composition comprising a therapeutically effective amount of an mTA disclosed herein. The disease or condition may be diabetes or obesity, or a medical condition associated with diabetes or obesity. The disease or condition may be non-alcoholic fatty liver disease (NAFLD), nonalcoholic steatohepatitis (NASH), or cardiovascular disease. The disease or condition may be short bowel syndrome (SBS). The disease or condition may be inflammatory bowel disease (IBD), inflammatory bowel syndrome (IBS), or psoriasis. The disease or condition may be Crohn's disease or ulcerative colitis. The disease or condition may be Alzheimer's disease, Parkinson's disease or Huntington's disease. The method of treating a disease or condition may further comprise administering to the subject one or more additional therapeutic agents.

The one or more additional therapeutic agents may be selected from a group consisting of other diabetes drugs, DPP4 inhibitors, SGLT2 inhibitors, hypoglycemic drugs and biguanidine drugs, insulin secretogogues and sulfonyl urea drugs, TZD drugs, insulin and insulin analogs, FGF21 and analogs, leptin or leptin analogs, amylin and amylin analogs, an anti-inflammatory drug, cyclosporine A or FK506, 5-ASA, and a statin, or any combination thereof.

### BRIEF DESCRIPTION OF THE DRAWINGS

The novel features of the invention are set forth with particularity in the appended claims. The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1A-C depict exemplary peptide lipid conjugates.

FIG. 2A-H depict exemplary staple-lipid constructs.

FIG. 3A-H depict exemplary peptide lipid conjugates.

FIG. 4 depicts amide-based macrocyclization.

FIG. 5 depicts the structure of mTA4 (SEQ ID NO: 31).

FIG. 6 depicts the structure of mTA6 (SEQ ID NO: 32).

FIG. 7 depicts the structure of mTA7 (SEQ ID NO: 33).

FIG. 8A depicts the in vitro activity of Ex-4, mTA4 and mTA6 in GLP-1R receptor-mediated CRE Luc reporter assay.

FIG. 8B depicts the in vitro activity of mTA4 and mTA6 in GCGR receptor-mediated CRE Luc reporter assay.

FIG. 8C depicts the in vitro activity of mTA4 and GLP-2 (G) in GLP-2R receptor-mediated CRE Luc reporter assay.

FIG. 9A depicts the plasma concentration of mTA4 in CD1 mice treated by i.v. or s.c. injections. Peptide concentrations in plasma were determined by in vitro GLP-1R or GLP-2R activity assay.

FIG. 9B depicts the plasma concentration of mTA7 in CD1 mice treated by i.v. or s.c. injections. Peptide concentrations in plasma were determined by in vitro GLP-1R or GLP-2R activity assay.

FIG. 10A depicts the effect on OGTT (Oral Glucose Tolerance Test) in DIO mice after of 2 weeks of treatment with mTA4 (s.c.).

FIG. 10B depicts the AUC of mTA4 on day 14 after.

FIG. 11A depicts the effect on body weight in DIO mice after of 2 weeks of treatment with mTA4 (s.c.).

FIG. 11B depicts the effect on food intake in DIO mice after of 10 dyas of treatment with mTA4 (s.c.).

FIG. 12 depicts the body weight gain by mTA7 after twice daily s.c. administrations in the chronic DSS model.

FIG. 13A depicts the colon length following treatment with mTA7.

FIG. 13B depicts the colon weight following treatment with mTA7.

FIG. 13C depicts the ratio between colon weight/length following treatment with mTA7.

FIG. 14A depicts the small intestine length following treatment with mTA7.

FIG. 14B depicts the small intestine weight following treatment with mTA7.

FIG. 14C depicts the ratio between small intestine weight/length following treatment with mTA7.

### DETAILED DESCRIPTION OF THE INVENTION

Disclosed herein, in some embodiments, are compounds comprising: a therapeutic peptide and a staple, wherein the staple is conjugated to each of two amino acids of the therapeutic peptide by the formation of an amide with an amine-containing sidechain of each of the two amino acids. A first region of a staple may be conjugated to a first amino acid of the therapeutic peptide and a second region of a staple may be conjugated to a second amino acid of a therapeutic peptide. The staple may stabilize the therapeutic peptide. A property of the therapeutic peptide in the composition may be different than a respective property of the therapeutic peptide alone. The property may be selected from an absorption rate constant, an absorption efficiency, an elimination rate constant, a half-life, a binding affinity, a binding efficiency, a disassociation constant, a target selectivity and a potency and in vivo efficacy. A therapeutic effect of the compound may be greater than a therapeutic effect of the therapeutic peptide alone. A therapeutic effect of the compound may be longer-lasting than a therapeutic effect of the therapeutic peptide alone.

Disclosed herein, in some embodiments, are compounds comprising: a therapeutic peptide, a staple and a non-peptide molecule, wherein the staple is conjugated to each of two amino acids of the therapeutic peptide by the formation of an amide with an amine-containing sidechain of each of the two amino acids; and the non-peptide molecule is conjugated to the staple. A first region of a staple may be conjugated to a first amino acid of the therapeutic peptide and a second region of a staple may be conjugated to a second amino acid of a therapeutic peptide. The non-peptide molecule may be conjugated to the staple such that the non-peptide molecule is distal to an active site or binding site of the therapeutic peptide. The staple may stabilize the therapeutic peptide while providing a conjugation site for the non-peptide molecule, such that the non-peptide molecule does not hinder and/or interfere with the therapeutic peptide binding to a target. A property of the therapeutic peptide in the composition may be different than a respective property of the therapeutic peptide alone. A property of the therapeutic peptide in the composition may be different than a respective property of the therapeutic peptide that is conjugated to the non-peptide molecule. The property may be selected from an absorption rate constant, an absorption efficiency, an elimination rate constant, a half-life, a binding affinity, a binding efficiency, a disassociation constant, a target selectivity and a potency and in vivo efficacy. A therapeutic effect of the compound may be greater than a therapeutic effect of the therapeutic peptide alone. A therapeutic effect of the compound may be greater than a therapeutic effect of a respective therapeutic peptide that is conjugated to the non-peptide molecule. A therapeutic effect of the compound may be longer-lasting than a therapeutic effect of the therapeutic peptide alone. A therapeutic effect of the composition may be longer-lasting than a therapeutic effect of a respective therapeutic peptide that is conjugated to the non-peptide molecule. The staple may stabilize the therapeutic peptide. The non-peptide molecule may extend a half-life of the therapeutic peptide. The non-peptide molecule may comprise a lipid moiety. The non-peptide molecule may comprise a polyethylene glycol unit.

Disclosed herein are modified therapeutic agents (mTAs). Generally, the mTA may comprise a therapeutic agent (TA), a staple, and a half-life extending molecule (HEM). The TA may be a modified or unmodified therapeutic peptide. The TA may be a modified therapeutic peptide. The modified therapeutic peptide may be a derivative of a peptide selected from GLP-1, glucagon, oxyntomodulin, exendin-4, GLP-2, and GIP; the derivative being a peptide comprising one or more amino acid additions, deletions, or substitutions, or a combination thereof. The TA may be an unmodified therapeutic peptide. The unmodified therapeutic peptide may be selected from GLP-1, glucagon, oxyntomodulin, exendin-4, GLP-2, and GIP. The staple may be covalently attached to the TA. The HEM may be attached to the staple. The staple may be attached to the TA via two amino acid residues on the modified or unmodified therapeutic peptide. One or both of the two amino acid residues may be an amino acid addition or substitution on the modified therapeutic peptide. One or both of the two amino acid residues may be selected from the group consisting of lysine, ornithine, diaminobutyric acid, diaminopropionic acid, and homolysine. One or both of the two amino acid residues may be lysine. The two amino acid residues may be at least about 4 amino acid residues apart. The two amino acid residues may be at least about 7 amino acid residues apart. The two amino acid residues may be at least about 11 amino acid residues apart. The HEM may comprise a lipid, a polyglycol region, or a combination of both. The HEM may comprise a lipid. The HEM may comprise a lipid and a polyglycol region. The HEM may comprise a polyglycol region. The HEM may comprise a peptide or protein. The lipid may be selected from a group consisting of sterols, sterol derivatives, bile acids, vitamin E derivatives, fatty di-acids, fatty acids, fatty amides, fatty amines, and fatty alcohols, and derivatives thereof. The polyglycol region may comprise one or more polyethylene glycol units, polypropylene glycol units, or polybutylene glycol units, or a combination thereof. The mTA may have more than one staple. The mTA may have more than one HEM. The mTA may have a longer half-life than the half-life of the unmodified therapeutic peptide alone. The mTA may have a higher potency than the potency of the unmodified therapeutic peptide alone. The mTA may have a higher target selectivity than the target selectivity of the unmodified therapeutic peptide alone. The mTA may have a higher binding affinity than the binding affinity of the unmodified therapeutic peptide alone.

Disclosed herein are peptide lipid conjugates (PLCs) which comprise one or more therapeutic agents (TAs), one or more staples that are directly attached to the one or more TAs; and one or more half-life extending molecules (HEMs) directly attached to the one or more staples. The one or more HEMs may comprise one or more lipids. The one or more HEMS may further comprise one or more polyethylene glycol subunits, wherein the one or more lipids are attached to the one or more polyethylene glycol subunits. Alternatively, the one or more HEMS may comprise one or more polyethylene glycol subunits. The one or more HEMS may comprise one or more peptides or proteins. The one or more HEMs may comprise one or more molecules selected from lipids, polyethylene glycol subunits, peptides, or proteins, or any combination thereof. The one or more HEMs may be a lipid. The PLCs may comprise two or more therapeutic agents. The PLCs may comprise two or more staples. The PLCs may comprise two or more HEMs. The PLCs may comprise a plurality of HEMs attached to the one or more staples.

Generally, the PLCs comprise one or more lipids attached to one or more peptide conjugates (PCs). The PLCs may comprise (a) one or more lipids; and (b) one or more peptide conjugates (PCs), each of the one or more peptide conjugates (PCs) comprising a peptide region comprising one or more peptide therapeutic agents (TA) and a staple region comprising one or more staples, the one or more staples connect two or more residues in the peptide region by the formation of an amide with an amine-containing sidechain from each of the two or more residues, wherein the one or more lipids are attached to the one or more staples. The two or more residues in the peptide region may comprise lysine, ornithine, diaminobutyric acid, diaminopropionic acid, or homolysine. The two or more residues in the peptide region may comprise lysine.

Disclosed herein are compositions comprising one or more mTAs as disclosed herein.

Disclosed herein are compositions comprising one or more PLCs as disclosed herein.

Further disclosed herein are compositions comprising one or more peptide lipid conjugates (PLCs) of Formula (I).

Disclosed herein are methods for treating a disease or condition in a subject in need thereof, the method comprising administering to the subject one or more mTAs disclosed herein.

Disclosed herein are methods for treating a disease or condition in a subject in need thereof, the method comprising administering to the subject one or more PLCs disclosed herein.

Before the present methods, kits and compositions are described in greater detail, it is to be understood that this invention is not limited to particular method, kit or composition described, as such may, of course, vary. It is also to be understood that the terminology used herein is for the purpose of describing particular embodiments only, and is not intended to be limiting, since the scope of the present invention will be limited only by the appended claims. Examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Methods and compositions are provided for producing mTAs and PLCs that extend the half-life of a therapeutic agent. These methods and compositions find therapeutic use in a number of diseases, for example, diabetes or obesity may be more effectively treated with a half-life extension molecule conjugated to a therapeutic peptide than by the therapeutic peptide alone. These and other objects, advantages, and features of the invention will become apparent to those persons skilled in the art upon reading the details of the compositions and methods as more fully described below.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Modified Therapeutic Agent (mTA)

Disclosed herein are modified therapeutic agents (mTAs) comprising a therapeutic agent (TA) and a staple. The TA may be a modified or unmodified therapeutic peptide. The TA may be covalently attached to the staple. The TA may be covalently attached to the staple via two amino acid residues on the modified or unmodified therapeutic peptide, wherein each of the two amino acid residues has an amine-containing sidechain for attachment to the staple through the formation of an amide. The two amino acid residues may be at least about 4 amino acid residues apart. The two amino acid residues may be at least about 7 amino acid residues apart. The two amino acid residues may be at least about 11 amino acid residues apart. Each of the two amino acids may be independently lysine, ornithine, diaminobutyric acid, diaminopropionic acid, or homolysine. The two amino acids may be lysine. The half-life of the mTA may be longer than the half-life of the modified or unmodified therapeutic peptide alone. The unmodified therapeutic peptide may be selected from GLP-1, glucagon, oxyntomodulin, exendin-4, GLP-2, and GIP. The modified therapeutic peptide may be a derivative of a peptide selected from GLP-1, glucagon, oxyntomodulin, exendin-4, GLP-2, and GIP; the derivative being a peptide comprising one or more amino acid additions, deletions, or substitutions, or a combination thereof.

Disclosed herein are modified therapeutic agents (mTAs) comprising a therapeutic agent (TA), a staple, and a half-life extending molecule (HEM). The TA may be a modified or unmodified therapeutic peptide. The TA may be covalently attached to the staple. The TA may be covalently attached to the staple via two amino acid residues on the modified or unmodified therapeutic peptide, wherein each of the two amino acid residues has an amine-containing sidechain for attachment to the staple through the formation of an amide. The two amino acid residues may be at least about 4 amino acid residues apart. The two amino acid residues may be at least about 7 amino acid residues apart. The two amino acid residues may be at least about 11 amino acid residues apart. Each of the two amino acids may be independently lysine, ornithine, diaminobutyric acid, diaminopropionic acid, or homolysine. The two amino acids may be lysine. The half-life of the mTA may be longer than the half-life of the modified or unmodified therapeutic peptide alone. The HEM may comprise a hydrophobic region, a hydrophilic region, or a combination thereof. The HEM may comprise a lipid, a polyglycol region, or a combination thereof. The lipid may be selected from a group consisting of sterols, sterol derivatives, bile acids, vitamin E derivatives, fatty di-acids, fatty acids, fatty amides, fatty amines, and fatty alcohols, and derivatives thereof. The polyglycol region comprises one or more polyethylene glycol units, polypropylene glycol units, or poly butylene glycol units, or a combination thereof. The HEM may comprise a protein or a peptide. The HEM may be covalently attached to the staple. The unmodified therapeutic peptide may be selected from GLP-1, glucagon, oxyntomodulin, exendin-4, GLP-2, and GIP. The modified therapeutic peptide may be a derivative of a peptide selected from GLP-1, glucagon, oxyntomodulin, exendin-4, GLP-2, and GIP; the derivative being a peptide comprising one or more amino acid additions, deletions, or substitutions, or a combination thereof. Non-limiting examples of mTAs include peptide lipid conjugates (PLCs).

Disclosed herein are modified therapeutic agents (mTAs) comprising a therapeutic agent (TA), a first staple, and a first HEM. The TA may be a modified or unmodified therapeutic peptide. The TA may be covalently attached to the first staple. The TA may be covalently attached to the first staple via two amino acid residues on the modified or unmodified therapeutic peptide, wherein each of the two amino acid residues has an amine-containing sidechain for attachment to the first staple through the formation of an amide. The two amino acid residues may be at least about 4 amino acid residues apart. The two amino acid residues may be at least about 7 amino acid residues apart. The two amino acid residues may be at least about 11 amino acid residues apart. Each of the two amino acids may be independently lysine, ornithine, diaminobutyric acid, diaminopropionic acid, or homolysine. The two amino acids may be lysine. The half-life of the mTA may be longer than the half-life of the modified or unmodified therapeutic peptide alone. The first HEM may comprise a hydrophobic region, a hydrophilic region, or a combination thereof. The first HEM may comprise a lipid, a polyglycol region, or a combination thereof. The lipid may be selected from a group consisting of sterols, sterol derivatives, bile acids, vitamin E derivatives, fatty di-acids, fatty acids, fatty amides, fatty amines, and fatty alcohols, and derivatives thereof. The polyglycol region comprises one or more polyethylene glycol units, polypropylene glycol units, or polybutylene glycol units, or a combination thereof. The first HEM may comprise a protein or a peptide. The first HEM may be covalently attached to the first staple. The mTA may further comprise a second staple. The mTA may further comprise a second HEM. The mTA may further comprise a second staple and a second HEM. The first staple and second staple may be the same or different. The first HEM and second HEM may be the same or different. The unmodified therapeutic peptide may be selected from GLP-1, glucagon, oxyntomodulin, exendin-4, GLP-2, and GIP. The modified therapeutic peptide may be a derivative of a peptide selected from GLP-1, glucagon, oxyntomodulin, exendin-4, GLP-2, and GIP; the derivative being a peptide comprising one or more amino acid additions, deletions, or substitutions, or a combination thereof. The mTA may be a PLC.

Further disclosed herein are modified therapeutic agents (mTAs) comprising a therapeutic agent (TA), a first staple, and a first HEM, wherein the therapeutic agent is a modified or unmodified therapeutic peptide that is covalently attached to the first staple via two amino acid residues on the modified or unmodified therapeutic peptide, and each of the two amino acid residues has an amine-containing sidechain for attachment to the first staple through the formation of an amide. The two amino acid residues may be at least about 4 amino acid residues apart. The two amino acid residues may be at least about 7 amino acid residues apart. The two amino acid residues may be at least about 11 amino acid residues apart. Each of the two amino acids may be independently lysine, ornithine, diaminobutyric acid, diaminopropionic acid, or homolysine. The two amino acids may be lysine. The half-life of the mTA may be longer than the half-life of the modified or unmodified therapeutic peptide alone. The first HEM may comprise a lipid, a polyglycol region, or a combination thereof. The lipid may be selected from a group consisting of sterols, sterol derivatives, bile acids, vitamin E derivatives, fatty di-acids, fatty acids, fatty amides, fatty amines, and fatty alcohols, and derivatives thereof. The polyglycol region comprises one or more polyethylene glycol units, polypropylene glycol units, or poly butylene glycol units, or a combination thereof. The first HEM may comprise a protein or a peptide. The first HEM may be covalently attached to the first staple. The mTA may further comprise a second staple. The mTA may further comprise a second HEM. The mTA may further comprise a second staple and a second HEM. The first staple and second staple may be the same or different. The first HEM and second HEM may be the same or different. The unmodified therapeutic peptide may be selected from GLP-1, glucagon, oxyntomodulin, exendin-4, GLP-2, and GIP. The modified therapeutic peptide may be a derivative of a peptide selected from GLP-1, glucagon, oxyntomodulin, exendin-4, GLP-2, and GIP; the derivative being a peptide comprising one or more amino acid additions, deletions, or substitutions, or a combination thereof. The mTA may be a PLC.

Further disclosed herein are modified therapeutic agents (mTAs) comprising a therapeutic agent (TA), a first staple, and a first HEM, wherein the therapeutic agent is a modified or unmodified therapeutic peptide that is covalently attached to the first staple via two amino acid residues on the modified or unmodified therapeutic peptide, each of the two amino acid residues has an amine-containing sidechain for attachment to the first staple through the formation of an amide, and the half-life of the mTA is longer than the half-life of the unmodified therapeutic peptide alone. The two amino acid residues may be at least about 4 amino acid residues apart. The two amino acid residues may be at least about 7 amino acid residues apart. The two amino acid residues may be at least about 11 amino acid residues apart. Each of the two amino acids may be independently lysine, ornithine, diaminobutyric acid, diaminopropionic acid, or homolysine. The two amino acids may be lysine. The first HEM may comprise a hydrophobic region, a hydrophilic region, or a combination thereof. The first HEM may comprise a lipid, a polyglycol region, or a combination thereof. The lipid may be selected from a group consisting of sterols, sterol derivatives, bile acids, vitamin E derivatives, fatty di-acids, fatty acids, fatty amides, fatty amines, and fatty alcohols, and derivatives thereof. The polyglycol region comprises one or more polyethylene glycol units, polypropylene glycol units, or poly butylene glycol units, or a combination thereof. The first HEM may comprise a protein or a peptide. The first HEM may be covalently attached to the first staple. The mTA may further comprise a second staple. The mTA may further comprise a second HEM. The mTA may further comprise a second staple and a second HEM. The first staple and second staple may be the same or different. The first HEM and second HEM may be the same or different. The unmodified therapeutic peptide may be selected from GLP-1, glucagon, oxyntomodulin, exendin-4, GLP-2, and GIP. The modified therapeutic peptide may be a derivative of a peptide selected from GLP-1, glucagon, oxyntomodulin, exendin-4, GLP-2, and GIP; the derivative being a peptide comprising one or more amino acid additions, deletions, or substitutions, or a combination thereof. The mTA may be a PLC.

Further disclosed herein are modified therapeutic agents (mTAs) comprising a therapeutic agent (TA), a first staple, and a first HEM, wherein the therapeutic agent is a modified or unmodified therapeutic peptide that is covalently attached to the first staple via two amino acid residues on the modified or unmodified therapeutic peptide, each of the two amino acid residues has an amine-containing sidechain for attachment to the first staple through the formation of an amide, and the first HEM is covalently attached to the first staple. The two amino acid residues may be at least about 4 amino acid residues apart. The two amino acid residues may be at least about 7 amino acid residues apart. The two amino acid residues may be at least about 11 amino acid residues apart. Each of the two amino acids may be independently lysine, ornithine, diaminobutyric acid, diaminopropionic acid, or homolysine. The two amino acids may be lysine. The half-life of the mTA may be longer than the half-life of the modified or unmodified therapeutic peptide alone. The first HEM may comprise a hydrophobic region, a hydrophilic region, or a combination thereof. The first HEM may comprise a lipid, a polyglycol region, or a combination thereof. The lipid may be selected from a group consisting of sterols, sterol derivatives, bile acids, vitamin E derivatives, fatty di-acids, fatty acids, fatty amides, fatty amines, and fatty alcohols, and derivatives thereof. The polyglycol region comprises one or more polyethylene glycol units, polypropylene glycol units, or polybutylene glycol units, or a combination thereof. The first HEM may comprise a protein or a peptide. The mTA may further comprise a second staple. The mTA may further comprise a second HEM. The mTA may further comprise a second staple and a second HEM. The first staple and second staple may be the same or different. The first HEM and second HEM may be the same or different. The unmodified therapeutic peptide may be selected from GLP-1, glucagon, oxyntomodulin, exendin-4, GLP-2, and GIP. The modified therapeutic peptide may be a derivative of a peptide selected from GLP-1, glucagon, oxyntomodulin, exendin-4, GLP-2, and GIP; the derivative being a peptide comprising one or more amino acid additions, deletions, or substitutions, or a combination thereof. The mTA may be a PLC.

Further disclosed herein are modified therapeutic agents (mTAs) comprising a therapeutic agent (TA), a first staple, and a first HEM, wherein the therapeutic agent is a modified or unmodified therapeutic peptide that is covalently attached to the first staple via two amino acid residues on the modified or unmodified therapeutic peptide, and each of the two amino acid residues has an amine-containing sidechain for attachment to the first staple through the formation of an amide; the first HEM is covalently attached to the first staple; and the half-life of the mTA is longer than the half-life of the unmodified therapeutic peptide alone. The two amino acid residues may be at least about 4 amino acid residues apart. The two amino acid residues may be at least about 7 amino acid residues apart. The two amino acid residues may be at least about 11 amino acid residues apart. Each of the two amino acids may be independently lysine, ornithine, diaminobutyric acid, diaminopropionic acid, or homolysine. The two amino acids may be lysine. The first HEM may comprise a hydrophobic region, a hydrophilic region, or a combination thereof. The first HEM may comprise a lipid, a polyglycol region, or a combination thereof. The lipid may be selected from a group consisting of sterols, sterol derivatives, bile acids, vitamin E derivatives, fatty di-acids, fatty acids, fatty amides, fatty amines, and fatty alcohols, and derivatives thereof. The polyglycol region comprises one or more polyethylene glycol units, polypropylene glycol units, or polybutylene glycol units, or a combination thereof. The first HEM may comprise a protein or a peptide. The mTA may further comprise a second staple. The mTA may further comprise a second HEM. The mTA may further comprise a second staple and a second HEM. The first staple and second staple may be the same or different. The first HEM and second HEM may be the same or different. The unmodified therapeutic peptide may be selected from GLP-1, glucagon, oxyntomodulin, exendin-4, GLP-2, and GIP. The modified therapeutic peptide may be a derivative of a peptide selected from GLP-1, glucagon, oxyntomodulin, exendin-4, GLP-2, and GIP; the derivative being a peptide comprising one or more amino acid additions, deletions, or substitutions, or a combination thereof. The mTA may be a PLC.

Further disclosed herein are modified therapeutic agents (mTAs) comprising a therapeutic agent (TA), a first staple, and a first HEM, wherein the therapeutic agent is a modified or unmodified therapeutic peptide that is covalently attached to the first staple via two lysine residues on the modified or unmodified therapeutic peptide and the first HEM is covalently attached to the first staple. The two lysines may be at least about 4 amino acid residues apart. The two lysines may be at least about 7 amino acid residues apart. The two lysines may be at least about 11 amino acid residues apart. The half-life of the mTA may be longer than the half-life of the modified or unmodified therapeutic peptide alone. The first HEM may comprise a hydrophobic region, a hydrophilic region, or a combination thereof. The first HEM may comprise a lipid, a polyglycol region, or a combination thereof. The lipid may be selected from a group consisting of sterols, sterol derivatives, bile acids, vitamin E derivatives, fatty di-acids, fatty acids, fatty amides, fatty amines, and fatty alcohols, and derivatives thereof. The polyglycol region comprises one or more polyethylene glycol units, polypropylene glycol units, or polybutylene glycol units, or a combination thereof. The first HEM may comprise a protein or a peptide. The mTA may further comprise a second staple. The mTA may further comprise a second HEM. The mTA may further comprise a second staple and a second HEM. The first staple and second staple may be the same or different. The first HEM and second HEM may be the same or different. The unmodified therapeutic peptide may be selected from GLP-1, glucagon, oxyntomodulin, exendin-4, GLP-2, and GIP. The modified therapeutic peptide may be a derivative of a peptide selected from GLP-1, glucagon, oxyntomodulin, exendin-4, GLP-2, and GIP; the derivative being a peptide comprising one or more amino acid additions, deletions, or substitutions, or a combination thereof. The mTA may be a PLC.

Further disclosed herein are modified therapeutic agents (mTAs) comprising a therapeutic agent (TA), a first staple, and a first HEM, wherein the therapeutic agent is a modified or unmodified therapeutic peptide that is covalently attached to the first staple via two amino acid residues on the modified or unmodified therapeutic peptide, and each of the two amino acid residues has an amine-containing sidechain for attachment to the first staple through the formation of an amide; the unmodified therapeutic peptide is selected from GLP-1, glucagon, oxyntomodulin, exendin-4, GLP-2, and GIP; the modified therapeutic peptide is a derivative of a peptide selected from GLP-1, glucagon, oxyntomodulin, exendin-4, GLP-2, and GIP, the derivative being a peptide comprising one or more amino acid additions, deletions, or substitutions, or a combination thereof; and the first HEM is covalently attached to the first staple. The two amino acid residues may be at least about 4 amino acid residues apart. The two amino acid residues may be at least about 7 amino acid residues apart. The two amino acid residues may be at least about 11 amino acid residues apart. Each of the two amino acids may be independently lysine, ornithine, diaminobutyric acid, diaminopropionic acid, or homolysine. The two amino acids may be lysine. The half-life of the mTA may be longer than the half-life of the modified or unmodified therapeutic peptide alone. The first HEM may comprise a hydrophobic region, a hydrophilic region, or a combination thereof. The first HEM may comprise a lipid, a polyglycol region, or a combination thereof. The lipid may be selected from a group consisting of sterols, sterol derivatives, bile acids, vitamin E derivatives, fatty di-acids, fatty acids, fatty amides, fatty amines, and fatty alcohols, and derivatives thereof. The polyglycol region comprises one or more polyethylene glycol units, polypropylene glycol units, or poly butylene glycol units, or a combination thereof. The first HEM may comprise a protein or a peptide. The mTA may further comprise a second staple. The mTA may further comprise a second HEM. The mTA may further comprise a second staple and a second HEM. The first staple and second staple may be the same or different. The first HEM and second HEM may be the same or different. The mTA may be a PLC.

Further disclosed herein are modified therapeutic agents (mTAs) comprising a therapeutic agent (TA), a first staple, and a first HEM, wherein the therapeutic agent is a modified or unmodified therapeutic peptide that is covalently attached to the first staple via two amino acid residues on the modified or unmodified therapeutic peptide, and each of the two amino acid residues has an amine-containing sidechain for attachment to the first staple through the formation of an amide; the first HEM is covalently attached to the first staple; and the first HEM comprises a lipid, a polyglycol region, or a combination thereof. The two amino acid residues may be at least about 4 amino acid residues apart. The two amino acid residues may be at least about 7 amino acid residues apart. The two amino acid residues may be at least about 11 amino acid residues apart. Each of the two amino acids may be independently lysine, ornithine, diaminobutyric acid, diaminopropionic acid, or homolysine. The two amino acids may be lysine. The half-life of the mTA may be longer than the half-life of the modified or unmodified therapeutic peptide alone. The lipid may be selected from a group consisting of sterols, sterol derivatives, bile acids, vitamin E derivatives, fatty di-acids, fatty acids, fatty amides, fatty amines, and fatty alcohols, and derivatives thereof. The polyglycol region comprises one or more polyethylene glycol units, polypropylene glycol units, or polybutylene glycol units, or a combination thereof. The first HEM may further comprise a protein or a peptide. The mTA may further comprise a second staple. The mTA may further comprise a second HEM. The mTA may further comprise a second staple and a second HEM. The first staple and second staple may be the same or different. The first HEM and second HEM may be the same or different. The unmodified therapeutic peptide may be selected from GLP-1, glucagon, oxyntomodulin, exendin-4, GLP-2, and GIP. The modified therapeutic peptide may be a derivative of a peptide selected from GLP-1, glucagon, oxyntomodulin, exendin-4, GLP-2, and GIP; the derivative being a peptide comprising one or more amino acid additions, deletions, or substitutions, or a combination thereof. The mTA may be a PLC.

Further disclosed herein are modified therapeutic agents (mTAs) comprising a therapeutic agent (TA), a first staple, and a first HEM, wherein the therapeutic agent is a modified or unmodified therapeutic peptide that is covalently attached to the first staple via two amino acid residues on the modified or unmodified therapeutic peptide, and each of the two amino acid residues has an amine-containing sidechain for attachment to the first staple through the formation of an amide; the unmodified therapeutic peptide is selected from GLP-1, glucagon, oxyntomodulin, exendin-4, GLP-2, and GIP; the modified therapeutic peptide is a derivative of a peptide selected from GLP-1, glucagon, oxyntomodulin, exendin-4, GLP-2, and GIP, the derivative being a peptide comprising one or more amino acid additions, deletions, or substitutions, or a combination thereof; the first HEM is covalently attached to the first staple; and the first HEM comprises a lipid, a polyglycol region, or a combination thereof. The two amino acid residues may be at least about 4 amino acid residues apart. The two amino acid residues may be at least about 7 amino acid residues apart. The two amino acid residues may be at least about 11 amino acid residues apart. Each of the two amino acids may be independently lysine, ornithine, diaminobutyric acid, diaminopropionic acid, or homolysine. The two amino acids may be lysine. The half-life of the mTA may be longer than the half-life of the modified or unmodified therapeutic peptide alone. The lipid may be selected from a group consisting of sterols, sterol derivatives, bile acids, vitamin E derivatives, fatty di-acids, fatty acids, fatty amides, fatty amines, and fatty alcohols, and derivatives thereof. The polyglycol region comprises one or more polyethylene glycol units, polypropylene glycol units, or polybutylene glycol units, or a combination thereof. The first HEM may further comprise a protein or a peptide. The mTA may further comprise a second staple. The mTA may further comprise a second HEM. The mTA may further comprise a second staple and a second HEM. The first staple and second staple may be the same or different. The first HEM and second HEM may be the same or different. The mTA may be a PLC.

Further disclosed herein are modified therapeutic agents (mTAs) comprising a therapeutic agent (TA), a first staple, and a first HEM, wherein the therapeutic agent is a modified or unmodified therapeutic peptide that is covalently attached to the first staple via two amino acid residues on the modified or unmodified therapeutic peptide, and each of the two amino acid residues has an amine-containing sidechain for attachment to the first staple through the formation of an amide; the unmodified therapeutic peptide is selected from GLP-1, glucagon, oxyntomodulin, exendin-4, GLP-2, and GIP; the modified therapeutic peptide is a derivative of a peptide selected from GLP-1, glucagon, oxyntomodulin, exendin-4, GLP-2, and GIP, the derivative being a peptide comprising one or more amino acid additions, deletions, or substitutions, or a combination thereof; the first HEM is covalently attached to the first staple; and the first HEM comprises a lipid, a polyglycol region, a peptide or protein, or a combination thereof. The two amino acid residues may be at least about 4 amino acid residues apart. The two amino acid residues may be at least about 7 amino acid residues apart. The two amino acid residues may be at least about 11 amino acid residues apart. Each of the two amino acids may be independently lysine, ornithine, diaminobutyric acid, diaminopropionic acid, or homolysine. The two amino acids may be lysine. The half-life of the mTA may be longer than the half-life of the modified or unmodified therapeutic peptide alone. The lipid may be selected from a group consisting of sterols, sterol derivatives, bile acids, vitamin E derivatives, fatty di-acids, fatty acids, fatty amides, fatty amines, and fatty alcohols, and derivatives thereof. The polyglycol region comprises one or more polyethylene glycol units, poly propylene glycol units, or polybutylene glycol units, or a combination thereof. The mTA may further comprise a second staple. The mTA may further comprise a second HEM. The mTA may further comprise a second staple and a second HEM. The first staple and second staple may be the same or different. The first HEM and second HEM may be the same or different. The mTA may be a PLC.

Further disclosed herein are modified therapeutic agents (mTAs) consisting essentially of a therapeutic agent (TA), a staple, and a half-life extending molecule (HEM). The TA may be a modified or unmodified therapeutic peptide. The TA may be covalently attached to the staple. The TA may be covalently attached to the staple via two amino acid residues on the modified or unmodified therapeutic peptide, wherein each of the two amino acid residues has an amine-containing sidechain for attachment to the staple through the formation of an amide. One or both of the two amino acid residues may be lysine, ornithine, diaminobutyric acid, diaminopropionic acid, or homolysine. The two amino acids may be lysine. The two amino acid residues may be at least about 4 amino acid residues apart. The two amino acid residues may be at least about 7 amino acid residues apart. The two amino acid residues may be at least about 11 amino acid residues apart. The half-life of the mTA may be longer than the half-life of the modified or unmodified therapeutic peptide alone. The HEM may comprise a lipid, a poly glycol region, or a combination thereof. The lipid may be selected from a group consisting of sterols, sterol derivatives, bile acids, vitamin E derivatives, fatty di-acids, fatty acids, fatty amides, fatty amines, and fatty alcohols, and derivatives thereof. The polyglycol region comprises one or more polyethylene glycol units, polypropylene glycol units, or polybutylene glycol units, or a combination thereof. The HEM may comprise a protein or a peptide. The HEM may be covalently attached to the staple. The unmodified therapeutic peptide may be selected from GLP-1, glucagon, oxyntomodulin, exendin-4, GLP-2, and GIP. The modified therapeutic peptide may be a derivative of a peptide selected from GLP-1, glucagon, oxyntomodulin, exendin-4, GLP-2, and GIP; the derivative being a peptide comprising one or more amino acid additions, deletions, or substitutions, or a combination thereof. Non-limiting examples of mTAs include peptide lipid conjugates (PLCs).

In some embodiments of the mTAs disclosed herein, the HEM is directly attached to the staple which is covalently attached to the modified or unmodified therapeutic peptide. In some embodiments, HEM attachment to the staple is preferred over HEM attachment to the modified or unmodified therapeutic peptide. In some embodiments, a first mTA, wherein the HEM is directly attached to the staple which is covalently attached to the modified or unmodified therapeutic peptide, has better activity than a second mTA, wherein the HEM is directly attached to the modified or unmodified therapeutic peptide.

### Half-Life Extending Molecules (HEMs)

Disclosed herein are modified therapeutic agents (mTAs) comprising a therapeutic agent (TA), a staple, and a half-life extending molecule (HEM), wherein the HEM is attached to the staple; the therapeutic agent is a modified or unmodified therapeutic peptide; and the half-life of the mTA is longer than the half-life of the modified or unmodified therapeutic peptide alone. The HEM may be non-proteinaceous or proteinaceous. The HEM may comprise a hydrophobic region, a hydrophilic region, or a combination thereof. The HEM may comprise a lipid, a polyglycol region, a peptide or a protein, or a combination thereof.

The HEM may be non-proteinaceous. The HEM may comprise a lipid, a poly glycol region, or a combination thereof. The HEM may comprise a hydrophobic region, a hydrophilic region, or a combination thereof. The HEM may be a non-proteinaceous polymer. Non-limiting examples of non-proteinaceous polymer include hydroxyalkyl starch, such as hydroxyethyl starch (HES), polyglycol, branched polyethylene glycols, polysialic acid, polyvinyl alcohol, polycarboxylate, poly(vinylpyrrolidone), dextran, or another biocompatible polymer.

The lipid may be selected from a group consisting of sterols, sterol derivatives, bile acids, vitamin E derivatives, fatty di-acids, fatty acids, fatty amides, fatty amines, and fatty alcohols, and derivatives thereof. The lipid may be a sterol or sterol derivative. The lipid may be a bile acid or derivative thereof. The lipid may be a vitamin E derivative. The lipid may be a fatty di-acid, fatty acid, fatty amide, fatty amine, or fatty alcohol. The fatty di-acid, fatty acid, fatty amide, fatty amine, or fatty alcohol may have 5-40 carbon atoms. The fatty di-acid, fatty acid, fatty amide, fatty amine, or fatty alcohol may have 5-30 carbon atoms. The fatty di-acid, fatty acid, fatty amide, fatty amine, or fatty alcohol may have 5-20 carbon atoms. The fatty di-acid, fatty acid, fatty amide, fatty amine, or fatty alcohol may have 6-40 carbon atoms. The fatty di-acid, fatty acid, fatty amide, fatty amine, or fatty alcohol may have 6-30 carbon atoms. The fatty di-acid, fatty acid, fatty amide, fatty amine, or fatty alcohol may have 7-40 carbon atoms. The fatty di-acid, fatty acid, fatty amide, fatty amine, or fatty alcohol may have 8-40 carbon atoms. The fatty di-acid, fatty acid, fatty amide, fatty amine, or fatty alcohol may have 9-40 carbon atoms. The fatty di-acid, fatty acid, fatty amide, fatty amine, or fatty alcohol may have 10-40 carbon atoms.

The lipid may be selected from a group consisting of propanoic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, myristic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid and hexatriacontanoic acid. The lipid may be selected from a group consisting of malonic acid, succinic acid, glutaric acid, adipic acid, pimelic acid, suberic acid, azelaic acid, sebacic acid, undecanedioic acid, dodecanedioic acid, tridecanedioic acid, tetradecanedioic acid, pentadecanedioic acid, hexadecanedioic acid, heptadecanedioic acid, octadecanedioic acid, and nonadecanedioic acid. The lipid may be selected from a group consisting of myristoleic acid, palmitoleic acid, sapienic acid, oleic acid, elaidic acid, vaccenic acid, linoleic acid, linoelaidic acid, α-linolenic acid, arachidonic acid, eicosapentanoic acid, erucic acid, docosahexaenoic acid. The lipid may be selected from a group consisting of cholesterol, 7α-OH cholesterol, 7α,25-dihydroxycholesterol, cholic acid, chenodeoxycholic acid, lithocholic acid, deoxycholic acid, glycocholic acid, glycodeoxycholic acid, glycolithocholic acid, and glycochenodeoxycholic acid.

The polyglycol region may comprise one or more polyethylene glycol units, polypropylene glycol units, or polybutylene glycol units, or a combination thereof. The polyglycol region may comprise one or more polyethylene glycol units. The polyglycol region may comprise one or more polypropylene glycol units. The polyglycol region may comprise one or more poly butylene glycol units.

The poly glycol region may comprise 10, 20, 30, 40, 50, 60, 70, 80, 90, 100, or more polyethylene glycol units, polypropylene glycol units, or polybutylene glycol units, or a combination thereof. The polyglycol region may comprise 10, 20, 30, 40, 50, 60, 70, 80, 90, 100, or more polyethylene glycol units. The polyglycol region may comprise 10, 20, 30, 40, 50, 60, 70, 80, 90, 100, or more polypropylene glycol units. The polyglycol region may comprise 10, 20, 30, 40, 50, 60, 70, 80, 90, 100, or more polybutylene glycol units.

The polyglycol region may comprise 100, 200, 300, 400, 500, 600, 700, 800, 900, 1000, or more polyethylene glycol units, polypropylene glycol units, or polybutylene glycol units, or a combination thereof. The polyglycol region may comprise 100, 200, 300, 400, 500, 600, 700, 800, 900, 1000, or more polyethylene glycol units. The polyglycol region may comprise 100, 200, 300, 400, 500, 600, 700, 800, 900, 1000, or more polypropylene glycol units. The polyglycol region may comprise 100, 200, 300, 400, 500, 600, 700, 800, 900, 1000, or more polybutylene glycol units.

The polyglycol region may comprise 1000, 2000, 3000, 4000, 5000, 6000, 7000, 8000, 9000, 10000, 11000, 12000, 13000, 14000, 15000, 16000, 17000, 18000, 19000, 20000, 25000, 30000, 35000, 40000, 45000, 50000, or more polyethylene glycol units, polypropylene glycol units, or poly butylene glycol units, or a combination thereof. The polyglycol region may comprise 1000, 2000, 3000, 4000, 5000, 6000, 7000, 8000, 9000, 10000, 11000, 12000, 13000, 14000, 15000, 16000, 17000, 18000, 19000, 20000, 25000, 30000, 35000, 40000, 45000, 50000, or more polyethylene glycol units. The polyglycol region may comprise 1000, 2000, 3000, 4000, 5000, 6000, 7000, 8000, 9000, 10000, 11000, 12000, 13000, 14000, 15000, 16000, 17000, 18000, 19000, 20000, 25000, 30000, 35000, 40000, 45000, 50000, or more polypropylene glycol units. The polyglycol region may comprise 1000, 2000, 3000, 4000, 5000, 6000, 7000, 8000, 9000, 10000, 11000, 12000, 13000, 14000, 15000, 16000, 17000, 18000, 19000, 20000, 25000, 30000, 35000, 40000, 45000, 50000, or more polybutylene glycol units.

The polyglycol region may comprise a molecular weight of 500-50,000 daltons. The polyglycol region may comprise a molecular weight of 500-40,000 daltons. The polyglycol region may comprise a molecular weight of 500-30,000 daltons. The polyglycol region may comprise a molecular weight of 500-20,000 daltons. The polyglycol region may comprise a molecular weight of 500, 600, 700, 800, 900, 1000, 2000, 3000, 4000, 5000, 6000, 7000, 8000, 9000, 10000, 15000, 20000, 25000, 30000, 35000, 40000, or 45000 daltons or more, including increments therein.

The HEM may comprise a peptide or protein. Non-limiting examples include serum albumin, transferrin, or the Fc domain of immunoglobulins, or variants thereof. Variants may occur naturally or be non-naturally occurring. Non-naturally occurring variants may be produced using mutagenesis techniques known in the art. Variants may comprise one or more conservative or non-conservative amino acid substitutions, deletions, or additions, or a combination thereof. The HEM may comprise an extended recombinant polypeptide (XTEN).

### Peptide Lipid Conjugate (PLC)

Disclosed herein are peptide lipid conjugates (PLCs) comprising one or more lipids and one or more peptide conjugates, each of the one or more peptide conjugates (PCs) comprising (a) one or more peptide regions comprising therapeutic agents (TAs); and (b) one or more staples, the one or more staples connecting two or more residues in the peptide region by the formation of an amide with an amine-containing sidechain from each of the two or more residues; wherein the one or more lipids are attached to the one or more staples. The lipid conjugate may further comprise one or more polyethylene glycol subunits. The one or more lipids may be pegylated. At least one of the two or more residues in the peptide region may be lysine, ornithine, diaminobutyric acid, diaminopropionic acid, or homolysine. The two or more residues in the peptide region may comprise lysine. The two or more residues may be at least about 4 amino acids apart. The two or more residues may be at least about 7 amino acids apart. The two or more residues may be at least about 11 amino acids apart.

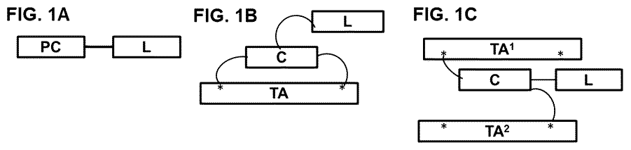

FIG. 1A-C depict schematics of exemplary peptide lipid conjugates. FIG. 1A depicts a peptide lipid conjugate comprising a peptide conjugate (PC) attached to a lipid (L). FIG. 1B depicts a peptide lipid conjugate comprising (a) a peptide conjugate comprising a staple (C) attached to two amino acid residues with amine-containing side chains (*) on a peptide therapeutic agent (TA); and (b) a lipid (L), wherein the lipid is attached to the staple. FIG. 1C depicts a peptide lipid conjugate comprising (a) a peptide conjugate comprising a staple (C) attached to two amino acid residues with amine-containing sidechains (*) on two peptide therapeutic agents (TA¹ and TA²); and (b) a lipid (L), wherein the lipid is attached to the staple. Each of the amino acid residues may independently be lysine, ornithine, diaminobutyric acid, diaminopropionic acid, or homolysine.

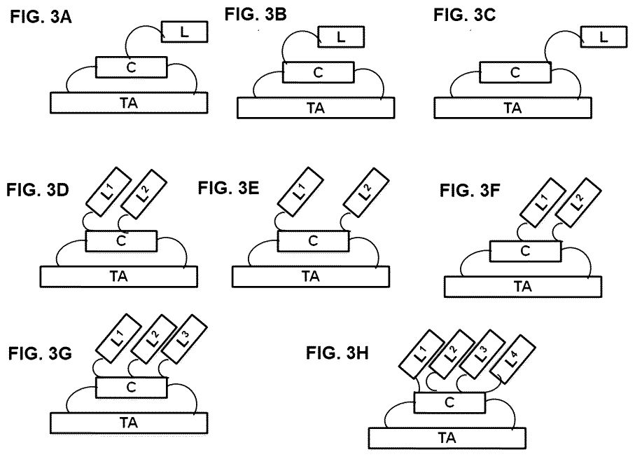

Additional exemplary peptide lipid conjugates are depicted in FIG. 3A-H.

FIG. 3A-C depict a peptide lipid conjugate comprising (a) a peptide conjugate comprising a staple (C) attached to a therapeutic agent (TA); and (b) a lipid (L). The lipid may be attached to any portion of the staple (C). For example, as shown in FIGS. 3A and 3C, the lipid may be attached to one end of the staple (C). As shown in FIG. 3B, the lipid may be attached to an internal region of the staple. FIG. 3D-F depict a peptide lipid conjugate comprising (a) a peptide conjugate comprising a staple (C) attached to a therapeutic agent (TA); and (b) two lipids (L¹ and L²). The two lipids may be attached to any portion of the staple. The two lipids may be attached to one or more ends of the staple. The two lipids may be attached to an internal region of the staple. The two lipids may be attached to an end of the staple and to an internal region of the staple. As shown in FIG. 3D, the first lipid (L¹) is attached to one end of the staple and the second lipid (L²) is attached to an internal region of the staple. As shown in FIG. 3E, the first lipid (L¹) and the second lipid (L²) are attached to opposite ends of the staple. As shown in FIG. 3F, the first lipid (L¹) is attached to an internal region of the staple and the second lipid (L²) is attached to one end of the staple. The PLCs disclosed herein may comprise (a) a peptide conjugate comprising a staple (C) and a therapeutic agent (TA); and (b) a plurality of lipids (L¹ . . . . Ln). FIG. 3G depicts a PLC comprising (a) a peptide conjugate comprising a staple (C) attached to a therapeutic agent (TA); and (b) three lipids (L¹, L² and L³). As shown in FIG. 3G, all three lipids are attached to various regions within the staple. FIG. 3H depicts a PLC comprising (a) a peptide conjugate comprising a staple (C) attached to a therapeutic agent (TA); and (b) four lipids (L¹, L², L³ and L⁴). As shown in FIG. 3H, all four lipids are attached to various regions within the staple.

The PLCs disclosed herein may have the structure:

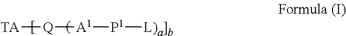

wherein:
TA is the therapeutic agent;
each Q is the same or different, and is a staple connected to two amino acid residues on the TA through the formation of an amide group with each of the two amino acid residues;
each A¹ is the same or different, and is a chemical group linking Q and P¹;
each P¹ is a bond or -PEG-A²-;
each PEG is the same or different, and is a chemical group comprising one or more polyethylene glycol subunits;
each A² is the same or different, and is a chemical group linking PEG and L;
each L is the same or different, and is a lipid derivative;
a is 1, 2, 3, 4, 5, 6, 7, 8, 9, or 10; and
b is 1, 2, 3, 4, 5, 6, 7, 8, 9, or 10.

In some embodiments of a PLC of Formula (I) disclosed herein, b is 1. In some embodiments of a PLC of Formula (I) disclosed herein, b is 2. In some embodiments of a PLC of Formula (I) disclosed herein, b is 3. In some embodiments of a PLC of Formula (I) disclosed herein, b is 4. In some embodiments of a PLC of Formula (I) disclosed herein, b is 5. In some embodiments of a PLC of Formula (I) disclosed herein, b is 6. In some embodiments of a PLC of Formula (I) disclosed herein, b is 7. In some embodiments of a PLC of Formula (I) disclosed herein, b is 8. In some embodiments of a PLC of Formula (I) disclosed herein, b is 9. In some embodiments of a PLC of Formula (I) disclosed herein, b is 10.

In some embodiments of a PLC of Formula (I) disclosed herein, Q is

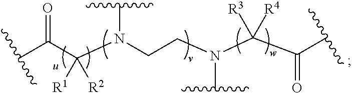

wherein
each R¹, R², R³, and R⁴ are independently selected from H, halo, CN, —SR⁵, alkyl, cycloalkyl, haloalkyl, —NR⁵R⁵, and —OR⁵;
each R⁵ is independently selected from H, alkyl, haloalkyl, arylalkyl, and heteroalkyl;
u is 1, 2, 3, 4, 5, 6, 7, 8, 9, or 10;
v is 0, 1, 2, 3, 4, 5, 6, 7, 8, or 9; and
w is 1, 2, 3, 4, 5, 6, 7, 8, 9, or 10.

In some embodiments of a PLC of Formula (I) disclosed herein, a is 1. In some embodiments of a PLC of Formula (I) disclosed herein, a is 2. In some embodiments of a PLC of Formula (I) disclosed herein, a is 3. In some embodiments of a PLC of Formula (I) disclosed herein, a is 4. In some embodiments of a PLC of Formula (I) disclosed herein, a is 5. In some embodiments of a PLC of Formula (I) disclosed herein, a is 6. In some embodiments of a PLC of Formula (I) disclosed herein, a is 7. In some embodiments of a PLC of Formula (I) disclosed herein, a is 8. In some embodiments of a PLC of Formula (I) disclosed herein, a is 9. In some embodiments of a PLC of Formula (I) disclosed herein, a is 1 or 2. In some embodiments of a PLC of Formula (I) disclosed herein, a is 1 and b is 1.

In some embodiments of a PLC of Formula (I) disclosed herein, Q is

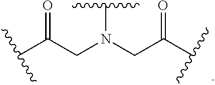 [SMILES: *C(=O)CN(*)CC(*)=O]

In some embodiments described of a PLC of Formula (I) disclosed herein, P¹ is-PEG-A².

In some embodiments of a PLC of Formula (I) disclosed herein, PEG is selected from:

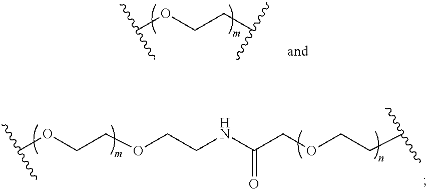

wherein
m and n are independently 1, 2, 3, 4, 5, 6, 7, 8, 9, 10, 11, 12, 13, 14, 15, 16, 17, 18, 19, or 20.

In some embodiments of a PLC of Formula (I) disclosed herein, PEG is selected from:

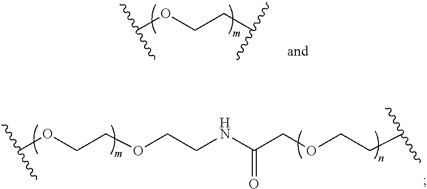

wherein
m and n are independently 1, 2, 3, or 4.

In some embodiments of a PLC of Formula (I) disclosed herein, PEG is

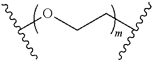

and m is 2.

In some embodiments of a PLC of Formula (I) disclosed herein, PEG is

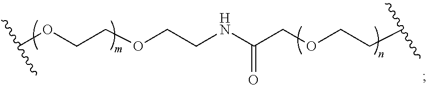

m is 2 and n is 1.

In some embodiments of a PLC of Formula (I) disclosed herein,
A¹ is selected from

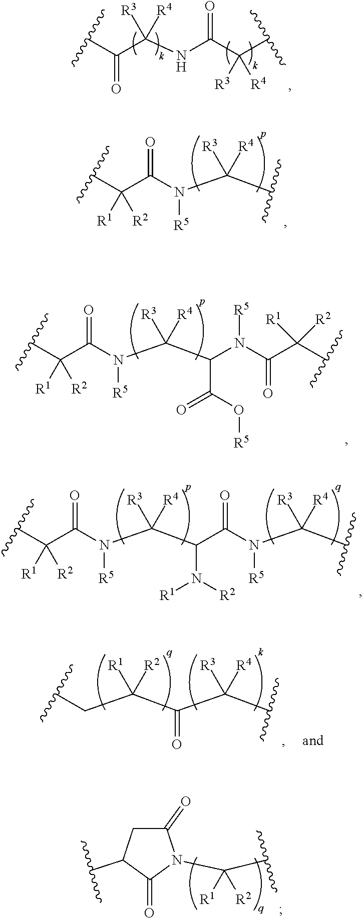

wherein
each R¹, R², R³, and R⁴ are independently selected from H, halo, CN, —SR⁵, alkyl, cycloalkyl, haloalkyl, —NR⁵R⁵, and —OR⁵;
each R⁵ is independently selected from H, alkyl, haloalkyl, arylalkyl, and heteroalkyl;
k is 1, 2, 3, 4, 5, 6, 7, 8, 9, or 10;
p is 2, 3, 4, 5, 6, 7, 8, 9, or 10; and
q is 2, 3, 4, 5, 6, 7, 8, 9, or 10.

In some embodiments of a PLC of Formula (I) disclosed herein, A¹ is

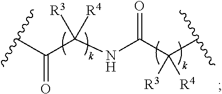

R³ and R⁴ are H; and; k are 2.

In some embodiments described of a PLC of Formula (I) disclosed herein,
A² is selected from a bond,

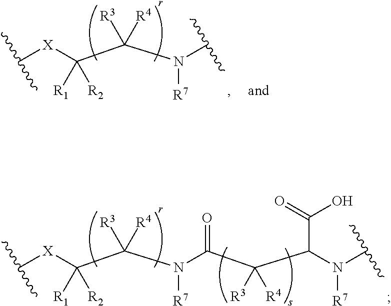

X is a bond, NR⁵, S, or O;
R¹, R², R³, and R⁴ are independently selected from H, halo, CN, —SR⁵, alkyl, cycloalkyl, haloalkyl, —NR⁵R⁵, and —OR⁵;
R⁵ is H, alkyl, haloalkyl, arylalkyl, or heteroalkyl;
each R⁷ is independently selected from H, alkyl, haloalkyl, arylalkyl, and heteroalkyl;
r is 1, 2, 3, 4, 5, 6, 7, 8, 9, or 10;
s is 1, 2, 3, 4, or 5; and
ti s 0, 1, 2, 3, 4, or 5.

In some embodiments described of a PLC of Formula (I) disclosed herein, A² is

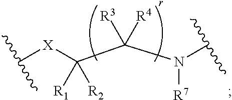

X is O; R¹, R², R³, and R⁴ are H; R⁷ is H; and r is 1;

In some embodiments described of a PLC of Formula (I) disclosed herein,
A² is

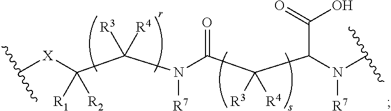

X is O; R¹, R², R³, and R⁴ are H; each R⁷ are H; r is 1; and s is 2. Each of the two amino acids of the PLC of Formula (I) may be independently selected from lysine, ornithine, diaminobutyric acid, diaminopropionic acid, and homolysine. The two amino acid residues may comprise lysine. The two amino acid residues may be at least about 4 amino acid residues apart. The two amino acid residues may be at least about 7 amino acid residues apart. The two amino acid residues may be at least about 11 amino acid residues apart. The PLC of Formula (I) may comprise one or more lipids. The PLC of Formula (I) may comprise one or more staples.

Disclosed herein are methods of producing a PLC of Formula (I), the method comprising reacting one or more staples with A³-P¹-L, wherein A³ is a reactive precursor to form A¹. A³ may be a haloacetamide, maleimide, benzyl halide, alkyl disulfide, or pyridyl disulfide. A³ may be a haloacetamide. A³ may a bromoacetamide. A³ may be an alkyl disulfide.

The peptide lipid conjugates (PLCs) may comprise (a) one or more lipids, the lipids selected from a group consisting of sterols, sterol derivatives, bile acids, vitamin E derivatives, fatty di-acids, fatty acids, fatty amides, and fatty alcohols; and (b) one or more peptide conjugates (PCs), each of the one or more peptide conjugate comprising a peptide region comprising one or more peptide therapeutic agents (TA) and a staple region comprising one or more staples, the one or more staples connect two or more residues in the peptide region by the formation of an amide with an amine-containing sidechain from each of the two or more residues, wherein the one or more lipids are attached to the one or more staples. Each of the two or more residues in the peptide region may independently comprise lysine, ornithine, diaminobutyric acid, diaminopropionic acid, or homolysine. The two amino acid residues may be at least about 4 amino acid residues apart. The two amino acid residues may be at least about 7 amino acid residues apart. The two amino acid residues may be at least about 11 amino acid residues apart.

The peptide lipid conjugates (PLCs) may comprise (a) one or more lipids; and (b) one or more peptide conjugates (PC), wherein the peptide conjugate comprising a peptide region comprising one or more peptide therapeutic agents (TA) and a staple region comprising one or more staples, the one or more staples connect two or more residues in the peptide region by the formation of an amide with an amine-containing sidechain from each of the two or more residues, the one or more peptide therapeutic agents comprising one or more oxyntomodulin, exendin-4, glucagon-like protein-1 (GLP-1), GLP-2, glucagon, GLP-1R/GIPR dual agonist, GLP-1R/GCGR dual agonist, or a derivative thereof, wherein the one or more lipids are attached to the one or more staples. Each of the two or more residues in the peptide region may independently comprise lysine, ornithine, diaminobutyric acid, diaminopropionic acid, or homolysine. The two amino acid residues may be at least about 4 amino acid residues apart. The two amino acid residues may be at least about 7 amino acid residues apart. The two amino acid residues may be at least about 11 amino acid residues apart.

The peptide lipid conjugates (PLCs) may comprise (a) one or more lipids; and (b) one or more peptide conjugates (PC), wherein the peptide conjugate comprising a peptide region comprising one or more peptide therapeutic agents (TA) and a staple region comprising one or more staples, the one or more staples connect two or more residues in the peptide region by the formation of an amide with an amine-containing sidechain from each of the two or more residues, the one or more peptide therapeutic agents comprising GLP-1, glucagon, oxyntomodulin, exendin-4, GLP-2, GIP, GLP-1R/GCGR dual agonist, GLP-1R/GIPR dual agonist, or GLP-1R/GCGR/GIPR tri-agonist, or a derivative thereof, wherein the one or more lipids are attached to the one or more staples. Each of the two or more residues in the peptide region may independently comprise lysine, ornithine, diaminobutyric acid, diaminopropionic acid, or homolysine. The two amino acid residues may be at least about 4 amino acid residues apart. The two amino acid residues may be at least about 7 amino acid residues apart. The two amino acid residues may be at least about 11 amino acid residues apart.

### Lipids

The mTAs or PLCs disclosed herein may comprise one or more lipids. The lipid may be attached to one or more peptide conjugates at a staple. The attachment of the one or more lipids to the one or more peptide conjugates may comprise a covalent attachment. The lipid may be attached to the peptide conjugate or lipid via one or more functional groups. The one or more functional groups may comprise a ketone. The one or more functional groups may comprise a carbonyl. The one or more functional groups may comprise an amide. Attachment of the lipid to the staple may enhance one or more pharmacokinetic properties of the TAs.

The one or more lipids may be fatty acids. Fatty acids may be fatty di-acids, fatty amines, fatty amides or fatty alcohols. Fatty acids may be saturated or unsaturated. Saturated fatty acids include, but are not limited to, lauric acid, myristic acid, palmitic acid, stearic acid, arachidic acid. Unsaturated fatty acids include, but are not limited to palmitoleic acid, oleic acid, linoleic acid, linolenic acid, erucic acid and arachidonic acid. Fatty acids may be short-chain fatty acids, medium chain fatty acids, long chain fatty acids or very long chain fatty acids. Fatty acids may be monounsaturated or polyunsaturated. Fatty acids may be omega fatty acids, essential fatty acids, partially hydrogenated fatty acids, cis-isomer fatty acids, or trans-isomer fatty acids. Fatty acids may be omega-3 fatty acids, omega-6 fatty acids or omega-9 fatty acids.

The fatty acid may comprise a chain of about 6, 7, 8, 9, 10, 11, 12, 13, 14, 15, 16, 17, 18, 19, 20, 21, 22, 23, 24, 25, 26 or more carbon atoms. The fatty acid may comprise a chain of 6-40 carbon atoms. The fatty acid may comprise a chain of 7-40 carbon atoms. The fatty acid may comprise a chain of 8-40 carbon atoms. The fatty acid may comprise a chain of 9-40 carbon atoms. The fatty acid may comprise a chain of 10-40 carbon atoms. The fatty acid may comprise a carbon chain further comprising 1, 2, 3, 4, 5, 6 or more double bonds. The fatty acid may be naturally occurring. The fatty acid may not be naturally occurring. The fatty acid may be synthesized.

The PLCs or mTAs disclosed herein may further comprise one or more fatty acids. The PLCs or mTAs disclosed herein may further comprise two or more fatty acids. The PLCs or mTAs disclosed herein may further comprise three or more fatty acids. The PLCs or mTAs disclosed herein may further comprise four or more fatty acids. The PLCs or mTAs disclosed herein may further comprise five or more fatty acids. The fatty acids may be different. The fatty acids may be the same.

The one or more lipids of the PLC or mTA may be selected from the group consisting of myristic acid, docosahexanoic acid, lithocholic acid ester, cholic acid, palmitic acid, and octadecanedioic acid. The one or more lipids of the PLC or mTA may be myristic acid. The one or more lipids of the PLC or mTA may be docosahexanoic acid. The one or more lipids of the PLC or mTA may be lithocholic acid ester. The one or more lipids of the PLC or mTA may be cholic acid. The one or more lipids of the PLC or mTA may be palmitic acid. The one or more lipids of the PLC or mTA may be myristic acid. The one or more lipids of the PLC or mTA may be octadecanedioic acid.

The PLCs or mTAs may comprise one or more sterols or sterol derivatives. The sterols or sterol derivatives may be selected from a group comprising cholesterol, 7α-OH cholesterol, 7α,25-dihydroxycholesterol, cholic acid, chenodeoxycholic acid, lithocholic acid, deoxycholic acid, glycocholic acid, glycodeoxycholic acid, glycolithocholic acid, and glycochenodeoxycholic acid.

The PLCs or mTAs may comprise one or more bile acids. The bile acids may be selected from a group comprising cholic acid, chenodeoxycholic acid, lithocholic acid, deoxycholic acid, glycocholic acid, glycodeoxycholic acid, glycolithocholic acid, and glycochenodeoxycholic acid.

The PLC or mTA may comprise one or more Vitamin E derivatives. The Vitamin E derivatives may be selected from a group comprising α-tocopherol, β-tocopherol, γ-tocopherol, δ-tocopherol, α-tocotrienol, β-tocotrienol, γ-tocotrienol and δ-tocotrienol.

### Pegylated Lipid

The PLCs disclosed herein may comprise one or more pegylated lipids. The mTAs disclosed herein may comprise one or more pegylated lipids.

The pegylated lipid may be attached to one or more peptide conjugates at one or more staples. The pegylated lipid may be attached to the staple via one or more functional groups. The one or more functional groups may comprise a ketone. The one or more functional groups may comprise a carbonyl. The one or more functional groups may comprise an amide. Attachment of the pegylated lipid to the staple may enhance one or more pharmacokinetic properties of the TAs.

A pegylated lipid may comprise at least one polyethylene glycol subunit. The connection between the lipid and the one or more polyethylene glycol subunits may be a direct bond or a linker (A²). Non-limiting examples of a linker between the lipid and the one or more polyethylene glycol subunits include:

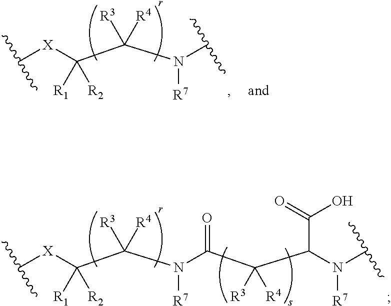

wherein
X is a bond, NR⁵, S, or O;
R¹, R², R³, and R⁴ are independently selected from H, halo, CN, —SR⁵, alkyl, cycloalkyl, haloalkyl, —NR⁵R⁵, and —OR⁵;
R⁵ is H, alkyl, haloalkyl, arylalkyl, or heteroalkyl;
each R⁷ is independently selected from H, alkyl, haloalkyl, arylalkyl, and heteroalkyl;
r is 1, 2, 3, 4, 5, 6, 7, 8, 9, or 10;
s is 1, 2, 3, 4, or 5; and
t is 0, 1, 2, 3, 4, or 5.

A pegylated lipid may have the structure P¹-L, wherein P¹ is-PEG-A²-; PEG is a chemical group comprising one or more polyethylene glycol subunits; A² is a chemical group linking PEG and L; and L is a lipid. PEG may be selected from:

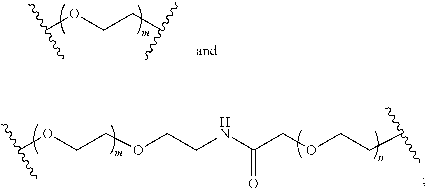

wherein
m and n are independently 1, 2, 3, 4, 5, 6, 7, 8, 9, 10, 11, 12, 13, 14, 15, 16, 17, 18, 19, or 20. A pegylated lipid may be connected to a staple through a linker.

### Lipid Derivatives

The lipid derivatives may be directly attached to a staple. Such attachment of the lipid derivative to the staple of a TA may enhance the pharmacokinetic properties of the TA. The mTAs and PLCs disclosed herein may comprise one or more lipid derivatives.

The lipid derivative may be attached to a peptide conjugate at a staple. The lipid derivative may be attached to one or more ends and/or internal regions of the staple. The lipid derivative may be attached to the staple of a peptide conjugate via one or more functional groups. The one or more functional groups may comprise a ketone. The one or more functional groups may comprise a carbonyl. The one or more functional groups may comprise an amide. Attachment of the lipid derivative to the staple may enhance the pharmacokinetic properties of the TAs.

Lipid derivatives may be pegylated. A pegylated lipid may comprise at least one polyethylene glycol subunit. The lipid derivatives may be not pegylated. Lipids may be broadly defined as hydrophobic or amphiphilic small molecules. Lipids may be naturally occurring or synthetic. Lipids may be eicosanoids, prostaglandins, leukotrienes, thromboxanes, wax esters, coenzyme A derivatives, fatty acid carnitines, fatty acid amides, ethanolamines, bile acids, vitamin E, vitamin A, vitamin D, vitamin K, fat-soluble vitamin derivatives, monoglycerides, diglycerides, triglycerides, phospholipids, phosphatidylcholine, glycerolipids, glycerols, glycerophospholipids, sphingolipids, saccharolipids, polyketides, sterols, sterol derivatives, sterol lipids, steroid hormones, prenol lipids, carotenoids, fatty acids, and fatty alcohols.

In one aspect, disclosed herein are lipid derivatives having the structure of A³-P¹-L, wherein:
A³ is a haloacetamide, maleimide, benzyl halide, alkyl disulfide, or pyridyl disulfide;
P¹ is a bond or -PEG-A²-;
PEG is a chemical group comprising one or more polyethylene glycol subunits;
A² is a chemical group linking PEG and L; and
L is a lipid selected from sterols, sterol derivatives, bile acids, vitamin E derivatives, fatty di-acids, fatty acids, fatty amides, and fatty alcohols.

In some embodiments described herein, PEG is selected from:

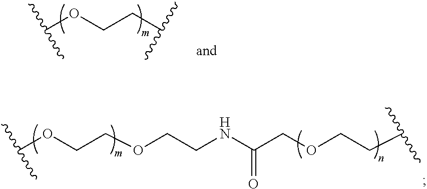

wherein
m and n are independently 1, 2, 3, 4, 5, 6, 7, 8, 9, 10, 11, 12, 13, 14, 15, 16, 17, 18, 19, or 20.

In some embodiments described herein,

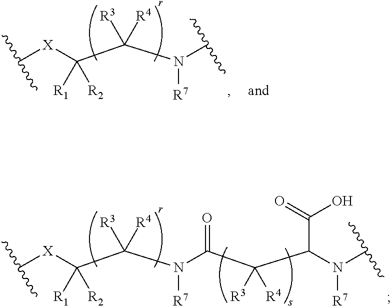

A² is selected from a bond,
X is a bond, NR⁵, S, or O;
R¹, R², R³, and R⁴ are independently selected from H, halo, CN, —SR⁵, alkyl, cycloalkyl, haloalkyl, —NR⁵R⁵, and —OR⁵;
R⁵ is H, alkyl, haloalkyl, arylalkyl, or heteroalkyl;
each R⁷ is independently selected from H, alkyl, haloalkyl, arylalkyl, and heteroalkyl;
r is 1, 2, 3, 4, 5, 6, 7, 8, 9, or 10;
s is 1, 2, 3, 4, or 5; and
t is 0, 1, 2, 3, 4, or 5.

Reaction of a lipid derivative with a staple precursor compound may produce a lipid staple intermediate. The lipid staple intermediate may subsequently be reacted with one or more therapeutic agents to form a PLC or mTA. Alternatively, reaction of a lipid derivative with a derivatizable functional group of the staple already attached to one or more therapeutic agents produces the PLC or mTA.

### Peptide Conjugate

The PLCs or mTAs disclosed herein may comprise one or more peptide conjugates. The peptide conjugates may comprise one or more staples connected to two or more amino acid residues on one or more therapeutic agents by the formation of an amide with an amine-containing sidechain from each of the two or more amino acid residues. The peptide conjugates may further comprise one or more additional staples. The one or more additional staples may be attached to one or more amino acid residues on the one or more therapeutic agents. The one or more amino acid residues may comprise a residue with an amine-containing sidechain on the one or more therapeutic agents. The one or more additional staples may be attached to the one or more staples.

The peptide conjugates may comprise one or more staples connected to one or more therapeutic agents, wherein at least two residues on the one or more therapeutic agents are connected to the one or more staples. The two residues may be on the same therapeutic agent. The two residues may be on different therapeutic agents. The two residues may be connected to the same staple. The two residues may be connected to different staples. Additional therapeutic agents may be attached to the one or more therapeutic agents or one or more staples. Attachment of the additional therapeutic agents to the one or more staples may occur via one or more amino acid residues. Each of the one or more amino acid residues may be independently lysine, ornithine, diaminobutyric acid, diaminopropionic acid, or homolysine. Additional staples may be attached to the one or more therapeutic agents or one or more staples. Attachment of the additional staples to the one or more therapeutic agents may occur via one or more amino acid residues. Each of the one or more amino acid residues may be independently lysine, ornithine, diaminobutyric acid, diaminopropionic acid, or homolysine.

### Staple

The PLCs or mTAs disclosed herein may comprise one or more staples. The PLCs or mTAs disclosed herein may comprise two or more staples. The PLCs or mTAs disclosed herein may comprise three or more staples. The PLCs or mTAs disclosed herein may comprise four or more staples. The PLCs or mTAs disclosed herein may comprise five or more staples. The PLCs or mTAs disclosed herein may comprise 6, 7, 8, 9, 10 or more staples.

The one or more staples may connect two or more amino acid residues in a peptide region of a peptide conjugate. At least one of the two or more amino acid residues may be lysine, ornithine, diaminobutyric acid, diaminopropionic acid, or homolysine. The two or more amino acid residues may both be lysine residues. The one or more staples may connect two or more lysine residues on the same TA. The one or more staples may connect two or more lysine residues on two or more TAs. Two or more staples may connect two or more lysine residues on the same TA. Two or more staples may connect two or more lysine residues on two or more TAs. At least one of the two or more amino acid residues may be a lysine residue. The one or more staples may connect two or more amino acid residues on the same TA. The one or more staples may connect two or more amino acid residues on two or more TAs. Two or more staples may connect two or more amino acid residues on the same TA. Two or more staples may connect two or more amino acid residues on two or more TAs. The two or more TAs may be the same. The two or more TAs may be different.

At least one staple may connect at least two amino acid residues on a therapeutic agent. The two amino acid residues may be adjacent. The two amino acid residues may be non-adjacent. The two amino acid residues may be at least about 1, 2, 3, 4, 5, 6, 7, 8, 9, 10, 11, 12, 13, 14, 15, 16, 17, 18, 19, 20 or more amino acid residues apart. The two amino acid residues may be at least about 4 amino acid residues apart. The two amino acid residues may be at least about 7 amino acid residues apart. The two amino acid residues may be at least about 11 amino acid residues apart. The two amino acid residues may be at least about 15 amino acid residues apart. The two amino acid residues may be at least about 19 amino acid residues apart.

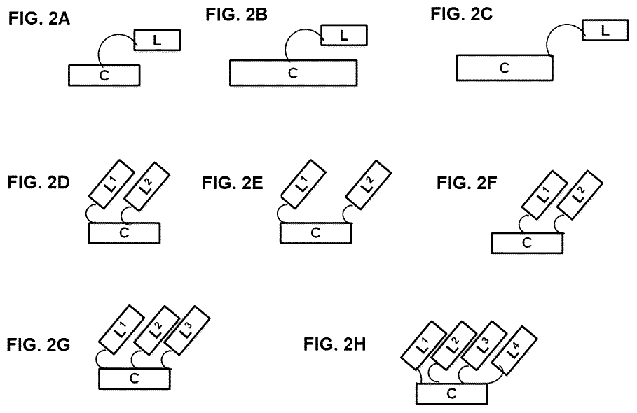

The staple may be conjugated to one or more lipids to produce a staple-lipid construct. Exemplary staple lipid constructs are shown in FIG. 2A-H. FIG. 2A-C depict a staple lipid construct comprising (a) a staple (C); and (b) a lipid (L). The lipid may be attached to any portion of the staple. As shown in FIG. 2A, the lipid (L) is attached to an internal region of the staple (C). As shown in FIGS. 2B and 2C, the lipid (L) is attached to one end of the staple (C). FIG. 2D-F depict a staple lipid construct comprising (a) a staple (C); and (b) two lipids (L¹ and L²). As shown in FIG. 2D, the first lipid (L¹) is attached to one end of the staple (C) and the second lipid (L²) is attached to an internal region of the staple (C). As shown in FIG. 2E, the first lipid (L¹) and the second lipid (L²) are attached opposite ends of the staple (C). As shown in FIG. 2F, the first lipid (L¹) is attached to an internal region of the staple (C) and the second lipid (L²) is attached to one end of the staple (C). The staple lipid construct may comprise (a) a staple (C); and (b) a plurality of lipids (L¹ . . . . Ln). FIG. 2G depicts a staple lipid construct comprising (a) a staple (C); and (b) three lipids (L¹, L² and L³). As shown in FIG. 2G, the three lipids (L¹, L² and L³) are attached to various regions within the staple (C). FIG. 2H depicts a staple lipid construct comprising (a) a staple (C); and (b) four lipids (L¹, L², L² and L³). As shown in FIG. 2G, the four lipids (L¹, L², L² and L³) are attached to various regions within the staple (C). The staple lipid constructs may further comprise one or more additional staples. The one or more additional staples may be attached to the staple, lipid, or a combination thereof. For example, the one or more additional staples may be attached to the staple and the lipid. Alternatively, the one or more additional staples are attached to the staple. The one or more additional staples may be attached to two or more staples. The one or more additional staples may be attached to the lipid. The one or more additional staples may be attached to the two or more lipids.

The staple may be prepared from a precursor compound comprising two or more chemical groups. The two or more chemical groups may each comprise a reactive carboxylic acid derivative. The two or more chemical groups may each comprise a reactive ester. The two or more chemical groups may each comprise an N-hydroxy succinimide ester. The two or more chemical groups may react with a nucleophilic amino acid residue on the same TA. The two or more chemical groups may react with a nucleophilic amino acid residue on two or more TAs. The nucleophilic amino acid residue may be an amino acid with an amine-containing sidechain. The nucleophilic amino acid residue may be lysine, ornithine, diaminobutyric acid, diaminopropionic acid, or homolysine.

The staple may be prepared from a precursor compound with two chemical groups, each of which reacts with a nucleophilic amino acid residue on the same TA.

The precursor may be selected from Table 2.

At least one or more lipids of the PLC of Formula (I) may be attached to the one or more staples to form a lipid staple precursor prior to forming the PLC. Each of the lipids of the PLC of Formula (I) may be attached to the one or more staples to form a lipid staple precursor prior to forming the PLC.

### Therapeutic Agent (TA)

The mTAs disclosed herein may comprise one or more therapeutic agents. The mTAs may comprise one therapeutic agent. The mTAs may comprise two therapeutic agents. The mTAs may comprise 3, 4, 5, 6, 7 or more therapeutic agents. The therapeutic agents may be different. The therapeutic agents may be the same.

The PCs disclosed herein may comprise one or more therapeutic agents. The PCs may comprise two or more therapeutic agents. The PCs may comprise 3, 4, 5, 6, 7 or more therapeutic agents. The two or more therapeutic agents may be different. The two or more therapeutic agents may be the same.

Exemplary TAs are depicted in Tables 3 and 4. Exemplary TAs may comprise a peptide sequence disclosed in Tables 3 and 4.

The TA may be selected from peptides listed Table 4, wherein X is an amino acid with an amine-containing side chain (e.g., lysine, ornithine, diaminobutyric acid, diaminopropionic acid, or homolysine).

The TA may be a hormone. Examples of hormones include, but are not limited to, peptide hormones, lipid and phospholipid-derived hormones, and monoamines. Peptide hormones generally consist of chains of amino acids. Examples of small peptide hormones include, but are not limited to thyrotropin-releasing hormone (TRH) and vasopressin. Peptides composed of scores or hundreds of amino acids are referred to as proteins. Examples of protein hormones include insulin and growth hormone. More complex protein hormones may bear carbohydrate side-chains and may be called glycoprotein hormones. Luteinizing hormone, follicle-stimulating hormone and thyroid-stimulating hormone are examples of glycoprotein hormones. Lipid and phospholipid-derived hormones are generally derived from lipids such as linoleic acid and arachidonic acid and phospholipids. Examples of protein hormones may comprise steroid hormones that may be derived from cholesterol and the eicosanoids. Examples of steroid hormones are testosterone and cortisol. Eicosanoids may comprise prostaglandins. Monoamines may be derived from aromatic amino acids like phenylalanine, tyrosine, and tryptophan by the action of aromatic amino acid decarboxylase enzymes. The TA may be leptin. The TA may be betatrophin. The TA may be a peptide agonist or peptide hormone. The peptide agonist or hormone may be exendin-4, glucagon, glucagon-like protein-1 (GLP-1), or oxyntomodulin. The TA may be exendin-4 (exenatide). The TA may be glucagon. The TA may be glucagon-like protein-1 (GLP-1). The TA may be oxyntomodulin. The TA may be GLP-2. The TA may be a GLP-1R/GIPR dual agonist. The TA may be a GLP-1R/GCGR dual agonist. The TA may be a GLP-1R/GCGR/GIPR tri-agonist. The TA may bind to a receptor. The receptor may be a GLP-1 receptor or glucagon receptor. The TA may be selected from GLP-1, glucagon, oxyntomodulin, exendin-4, GLP-2, GIP, GLP-1R/GCGR dual agonist, GLP-1R/GIPR dual agonist, and GLP-1R/GCGR/GIPR tri-agonist. The TA may be selected from GLP-1, glucagon, oxyntomodulin, exendin-4, GLP-2, and GIP.

The TA may be a growth factor. Growth factors may include, but are not limited to, cytokines and hormones. Examples of growth factors include, but are not limited to, adrenomedullin (AM), angiopoietin (Ang), autocrine motility factor, bone morphogenetic proteins (BMPs), brain-derived neurotrophic factor (BDNF), epidermal growth factor (EGF), erythropoietin (EPO), fibroblast growth factor (FGF), glial cell line-derived neurotrophic factor (GDNF), granulocyte colony-stimulating factor (G-CSF), granulocyte macrophage colony-stimulating factor (GM-CSF), growth differentiation factor-9 (GDF9), hepatocyte growth factor (HGF), hepatoma-derived growth factor (HDGF), insulin-like growth factor (IGF), migration-stimulating factor, myostatin (GDF-8), nerve growth factor (NGF) and other neurotrophins, platelet-derived growth factor (PDGF), thrombopoietin (TPO), transforming growth factor alpha (TGF-α), transforming growth factor beta (TGF-β), tumor necrosis factor-alpha (TNF-α) and vascular endothelial growth factor (VEGF). The TA may be fibroblast growth factor 21 (FGF21).

The TA may be a cell regulatory protein. The TA may be a cell regulatory protein of the transforming growth factor beta superfamily. The TA may be a member of the decapentaplegic-Vg related (DVR) related subfamily. The TA may be a member of the activin/inhibin subfamily. The TA may be a member of the TGF-beta subfamily. The TA may be a growth differentiation factor (GDF). The GDF may be GDF1, GDF2, GDF3, GDF5, GDF6, GFD8, GDF9, GDF10, GDF11, and GDF15. The TA may be growth differentiation factor 11 (GDF11).

The TA may be a protein. The protein may be a member of the angiopoietin-like family of secreted factors. The protein may be an angiopoietin-like protein (ANGPTL). Examples of ANGPTLs include, but are not limited to, ANGPTL1, ANGPTL2, ANGPTL3, ANGPTL4, ANGPTL5, ANGPTL6 and ANGPTL7. The TA may be ANGPTL3.

The TA may comprise an amino acid sequence selected from the group comprising SEQ ID NO: 1-6. The TA may comprise an amino acid sequence that is at least about 50% homologous to an amino acid sequence selected from the group comprising SEQ ID NO: 1-6. The TA may comprise an amino acid sequence that is at least about 55%, 60%, 65%, 70%, 75%, 80%, 85%, 90%, 92%, 95%, 97%, 99%, or 100% homologous to an amino acid sequence selected from the group comprising SEQ ID NO: 1-6. The TA may comprise an amino acid sequence that is at least about 70% homologous to an amino acid sequence selected from the group comprising SEQ ID NO: 1-6. The TA may comprise an amino acid sequence that is at least about 75% homologous to an amino acid sequence selected from the group comprising SEQ ID NO: 1-6. The TA may comprise an amino acid sequence that is at least about 80% homologous to an amino acid sequence selected from the group comprising SEQ ID NO: 1-6. The TA may comprise an amino acid sequence that is at least about 85% homologous to an amino acid sequence selected from the group comprising SEQ ID NO: 1-6. The TA may comprise an amino acid sequence that is at least about 90% homologous to an amino acid sequence selected from the group comprising SEQ ID NO: 1-6. The TA may comprise an amino acid sequence that is at least about 95% homologous to an amino acid sequence selected from the group comprising SEQ ID NO: 1-6.

The TA may comprise 20 or more consecutive amino acids from an amino acid sequence selected from the group comprising SEQ ID NO: 1-6. The TA may comprise 30, 40, 50, 60, 70, 80, 90, 100, 110, 120, 130 or more consecutive amino acids from an amino acid sequence selected from the group comprising SEQ ID NO: 1-6.

The TA may comprise an amino acid sequence selected from the group comprising SEQ ID NO: 7-30. The TA may comprise an amino acid sequence that is at least about 50% homologous to an amino acid sequence selected from the group comprising SEQ ID NO: 7-30. The TA may comprise an amino acid sequence that is at least about 55%, 60%, 65%, 70%, 75%, 80%, 85%, 90%, 92%, 95%, 97%, 99%, or 100% homologous to an amino acid sequence selected from the group comprising SEQ ID NO: 7-30. The TA may comprise an amino acid sequence that is at least about 70% homologous to an amino acid sequence selected from the group comprising SEQ ID NO: 7-30. The TA may comprise an amino acid sequence that is at least about 75% homologous to an amino acid sequence selected from the group comprising SEQ ID NO: 7-30. The TA may comprise an amino acid sequence that is at least about 80% homologous to an amino acid sequence selected from the group comprising SEQ ID NO: 7-30. The TA may comprise an amino acid sequence that is at least about 85% homologous to an amino acid sequence selected from the group comprising SEQ ID NO: 7-30. The TA may comprise an amino acid sequence that is at least about 90% homologous to an amino acid sequence selected from the group comprising SEQ ID NO: 7-30. The TA may comprise an amino acid sequence that is at least about 95% homologous to an amino acid sequence selected from the group comprising SEQ ID NO: 7-30.

The TA may comprise 10 or more consecutive amino acids from an amino acid sequence selected from the group comprising SEQ ID NO: 7-30. The TA may comprise 5, 6, 7, 8, 9, 10 or more consecutive amino acids from an amino acid sequence selected from the group comprising SEQ ID NO: 7-30.

The TA may comprise an amino acid sequence selected from the group consisting of SEQ ID NO: 7-30. The TA may comprise an amino acid sequence that is at least about 50% homologous to an amino acid sequence selected from the group consisting of SEQ ID NO: 7-30. The TA may comprise an amino acid sequence that is at least about 55%, 60%, 65%, 70%, 75%, 80%, 85%, 90%, 92%, 95%, 97%, 99%, or 100% homologous to an amino acid sequence selected from the group consisting of SEQ ID NO: 7-30. The TA may comprise an amino acid sequence that is at least about 70% homologous to an amino acid sequence selected from the group consisting of SEQ ID NO: 7-30. The TA may comprise an amino acid sequence that is at least about 75% homologous to an amino acid sequence selected from the group consisting of SEQ ID NO: 7-30. The TA may comprise an amino acid sequence that is at least about 80% homologous to an amino acid sequence selected from the group consisting of SEQ ID NO: 7-30. The TA may comprise an amino acid sequence that is at least about 85% homologous to an amino acid sequence selected from the group consisting of SEQ ID NO: 7-30. The TA may comprise an amino acid sequence that is at least about 90% homologous to an amino acid sequence selected from the group consisting of SEQ ID NO: 7-30. The TA may comprise an amino acid sequence that is at least about 95% homologous to an amino acid sequence selected from the group consisting of SEQ ID NO: 7-30.

The TA may comprise 10 or more consecutive amino acids from an amino acid sequence selected from the group consisting of SEQ ID NO: 7-30. The TA may comprise 5, 6, 7, 8, 9, 10 or more consecutive amino acids from an amino acid sequence selected from the group consisting of SEQ ID NO: 7-30.

The TA may comprise an amino acid sequence selected from the group comprising SEQ ID NO: 13, 15, and 25. The TA may comprise an amino acid sequence that is at least about 50% homologous to an amino acid sequence selected from the group comprising SEQ ID NO: 13, 15, and 25. The TA may comprise an amino acid sequence that is at least about 55%, 60%, 65%, 70%, 75%, 80%, 85%, 90%, 92%, 95%, 97%, 99%, or 100% homologous to an amino acid sequence selected from the group comprising SEQ ID NO: 13, 15, and 25. The TA may comprise an amino acid sequence that is at least about 70% homologous to an amino acid sequence selected from the group comprising SEQ ID NO: 13, 15, and 25. The TA may comprise an amino acid sequence that is at least about 75% homologous to an amino acid sequence selected from the group comprising SEQ ID NO: 13, 15, and 25. The TA may comprise an amino acid sequence that is at least about 80% homologous to an amino acid sequence selected from the group comprising SEQ ID NO: 13, 15, and 25. The TA may comprise an amino acid sequence that is at least about 85% homologous to an amino acid sequence selected from the group comprising SEQ ID NO: 13, 15, and 25. The TA may comprise an amino acid sequence that is at least about 90% homologous to an amino acid sequence selected from the group comprising SEQ ID NO: 13, 15, and 25. The TA may comprise an amino acid sequence that is at least about 95% homologous to an amino acid sequence selected from the group comprising SEQ ID NO: 13, 15, and 25.

The TA may comprise 10 or more consecutive amino acids from an amino acid sequence selected from the group comprising SEQ ID NO: 13, 15, and 25. The TA may comprise 5, 6, 7, 8, 9, 10 or more consecutive amino acids from an amino acid sequence selected from the group comprising SEQ ID NO: 13, 15, and 25.

The TA may comprise an amino acid sequence selected from the group consisting of SEQ ID NO: 13, 15, and 25. The TA may comprise an amino acid sequence that is at least about 50% homologous to an amino acid sequence selected from the group consisting of SEQ ID NO: 13, 15, and 25. The TA may comprise an amino acid sequence that is at least about 55%, 60%, 65%, 70%, 75%, 80%, 85%, 90%, 92%, 95%, 97%, 99%, or 100% homologous to an amino acid sequence selected from the group consisting of SEQ ID NO: 13, 15, and 25. The TA may comprise an amino acid sequence that is at least about 70% homologous to an amino acid sequence selected from the group consisting of SEQ ID NO: 13, 15, and 25. The TA may comprise an amino acid sequence that is at least about 75% homologous to an amino acid sequence selected from the group consisting of SEQ ID NO: 13, 15, and 25. The TA may comprise an amino acid sequence that is at least about 80% homologous to an amino acid sequence selected from the group consisting of SEQ ID NO: 13, 15, and 25. The TA may comprise an amino acid sequence that is at least about 85% homologous to an amino acid sequence selected from the group consisting of SEQ ID NO: 13, 15, and 25. The TA may comprise an amino acid sequence that is at least about 90% homologous to an amino acid sequence selected from the group consisting of SEQ ID NO: 13, 15, and 25. The TA may comprise an amino acid sequence that is at least about 95% homologous to an amino acid sequence selected from the group consisting of SEQ ID NO: 13, 15, and 25.

The TA may comprise 10 or more consecutive amino acids from an amino acid sequence selected from the group consisting of SEQ ID NO: 13, 15, and 25. The TA may comprise 5, 6, 7, 8, 9, 10 or more consecutive amino acids from an amino acid sequence selected from the group consisting of SEQ ID NO: 13, 15, and 25.

The TA may comprise the amino acid sequence SEQ ID NO: 13. The TA may comprise an amino acid sequence that is at least about 50% homologous to the amino acid sequence SEQ ID NO: 13. The TA may comprise an amino acid sequence that is at least about 55%, 60%, 65%, 70%, 75%, 80%, 85%, 90%, 92%, 95%, 97%, 99%, or 100% homologous to the amino acid sequence SEQ ID NO: 13. The TA may comprise an amino acid sequence that is at least about 70% homologous to the amino acid sequence SEQ ID NO: 13. The TA may comprise an amino acid sequence that is at least about 75% homologous to the amino acid sequence SEQ ID NO: 13. The TA may comprise an amino acid sequence that is at least about 80% homologous to the amino acid sequence SEQ ID NO: 13. The TA may comprise an amino acid sequence that is at least about 85% homologous to the amino acid sequence SEQ ID NO: 13. The TA may comprise an amino acid sequence that is at least about 90% homologous to the amino acid sequence SEQ ID NO: 13. The TA may comprise an amino acid sequence that is at least about 95% homologous to the amino acid sequence SEQ ID: 13.

The TA may comprise 10 or more consecutive amino acids from the amino acid sequence SEQ ID NO: 13. The TA may comprise 5, 6, 7, 8, 9, 10 or more consecutive amino acids from the amino acid sequence SEQ ID NO: 13.

The TA may comprise the amino acid sequence SEQ ID NO: 15. The TA may comprise an amino acid sequence that is at least about 50% homologous to the amino acid sequence SEQ ID NO: 15. The TA may comprise an amino acid sequence that is at least about 55%, 60%, 65%, 70%, 75%, 80%, 85%, 90%, 92%, 95%, 97%, 99%, or 100% homologous to the amino acid sequence SEQ ID NO: 15. The TA may comprise an amino acid sequence that is at least about 70% homologous to the amino acid sequence SEQ ID NO: 15. The TA may comprise an amino acid sequence that is at least about 75% homologous to the amino acid sequence SEQ ID NO: 15. The TA may comprise an amino acid sequence that is at least about 80% homologous to the amino acid sequence SEQ ID NO: 15. The TA may comprise an amino acid sequence that is at least about 85% homologous to the amino acid sequence SEQ ID NO: 15. The TA may comprise an amino acid sequence that is at least about 90% homologous to the amino acid sequence SEQ ID NO: 15. The TA may comprise an amino acid sequence that is at least about 95% homologous to the amino acid sequence SEQ ID: 15.

The TA may comprise 10 or more consecutive amino acids from the amino acid sequence SEQ ID NO: 15. The TA may comprise 5, 6, 7, 8, 9, 10 or more consecutive amino acids from the amino acid sequence SEQ ID NO: 15.

The TA may comprise the amino acid sequence SEQ ID NO: 25. The TA may comprise an amino acid sequence that is at least about 50% homologous to the amino acid sequence SEQ ID NO: 25. The TA may comprise an amino acid sequence that is at least about 55%, 60%, 65%, 70%, 75%, 80%, 85%, 90%, 92%, 95%, 97%, 99%, or 100% homologous to the amino acid sequence SEQ ID NO: 25. The TA may comprise an amino acid sequence that is at least about 70% homologous to the amino acid sequence SEQ ID NO: 25. The TA may comprise an amino acid sequence that is at least about 75% homologous to the amino acid sequence SEQ ID NO: 25. The TA may comprise an amino acid sequence that is at least about 80% homologous to the amino acid sequence SEQ ID NO: 25. The TA may comprise an amino acid sequence that is at least about 85% homologous to the amino acid sequence SEQ ID NO: 25. The TA may comprise an amino acid sequence that is at least about 90% homologous to the amino acid sequence SEQ ID NO: 25. The TA may comprise an amino acid sequence that is at least about 95% homologous to the amino acid sequence SEQ ID: 25.

The TA may comprise 10 or more consecutive amino acids from the amino acid sequence SEQ ID NO: 25. The TA may comprise 5, 6, 7, 8, 9, 10 or more consecutive amino acids from the amino acid sequence SEQ ID NO: 25.

The TA may comprise an amino acid sequence selected from the group comprising SEQ ID NO: 11, 16, 19, 25, and 29. The TA may comprise an amino acid sequence that is at least about 50% homologous to an amino acid sequence selected from the group comprising SEQ ID NO: 11, 16, 19, 25, and 29. The TA may comprise an amino acid sequence that is at least about 55%, 60%, 65%, 70%, 75%, 80%, 85%, 90%, 92%, 95%, 97%, 99%, or 100% homologous to an amino acid sequence selected from the group comprising SEQ ID NO: 11, 16, 19, 25, and 29. The TA may comprise an amino acid sequence that is at least about 70% homologous to an amino acid sequence selected from the group comprising SEQ ID NO: 11, 16, 19, 25, and 29. The TA may comprise an amino acid sequence that is at least about 75% homologous to an amino acid sequence selected from the group comprising SEQ ID NO: 11, 16, 19, 25, and 29. The TA may comprise an amino acid sequence that is at least about 80% homologous to an amino acid sequence selected from the group comprising SEQ ID NO: 11, 16, 19, 25, and 29. The TA may comprise an amino acid sequence that is at least about 85% homologous to an amino acid sequence selected from the group comprising SEQ ID NO: 11, 16, 19, 25, and 29. The TA may comprise an amino acid sequence that is at least about 90% homologous to an amino acid sequence selected from the group comprising SEQ ID NO: 11, 16, 19, 25, and 29. The TA may comprise an amino acid sequence that is at least about 95% homologous to an amino acid sequence selected from the group comprising SEQ ID NO: 11, 16, 19, 25, and 29.

The TA may comprise 10 or more consecutive amino acids from an amino acid sequence selected from the group comprising SEQ ID NO: 11, 16, 19, 25, and 29. The TA may comprise 5, 6, 7, 8, 9, 10 or more consecutive amino acids from an amino acid sequence selected from the group comprising SEQ ID NO: 11, 16, 19, 25, and 29.

The TA may comprise the amino acid sequence SEQ ID NO: 7. The TA may comprise an amino acid sequence that is at least about 50% homologous to the amino acid sequence SEQ ID NO: 7. The TA may comprise an amino acid sequence that is at least about 55%, 60%, 65%, 70%, 75%, 80%, 85%, 90%, 92%, 95%, 97%, 99%, or 100% homologous to the amino acid sequence SEQ ID NO: 7. The TA may comprise an amino acid sequence that is at least about 70% homologous to the amino acid sequence SEQ ID NO: 7. The TA may comprise an amino acid sequence that is at least about 75% homologous to the amino acid sequence SEQ ID NO: 7. The TA may comprise an amino acid sequence that is at least about 80% homologous to the amino acid sequence SEQ ID NO: 7. The TA may comprise an amino acid sequence that is at least about 85% homologous to the amino acid sequence SEQ ID NO: 7. The TA may comprise an amino acid sequence that is at least about 90% homologous to the amino acid sequence SEQ ID NO: 7. The TA may comprise an amino acid sequence that is at least about 95% homologous to the amino acid sequence SEQ ID: 7.

The TA may comprise 10 or more consecutive amino acids from the amino acid sequence SEQ ID NO: 7. The TA may comprise 5, 6, 7, 8, 9, 10 or more consecutive amino acids from the amino acid sequence SEQ ID NO: 7.

The TA may comprise one or more amino acid residues with amine-containing sidechains. The one or more amino acid residues with amine-containing sidechains may be used for connecting the staple to the TA. The one or more amino acid residues with amine-containing sidechains may be used for connecting a first TA to a second TA. The one or more amino acid residues with amine-containing sidechains may be naturally occurring in the TA. Alternatively, the one or more amino acid residues with amine-containing sidechains are introduced into the TA. The one or more amino acid residues with amine-containing sidechains may be inserted into the TA. The one or more amino acid residues with amine-containing sidechains may replace one or more amino acid residues in the TA. Methods for amino acid substitution and/or insertion are known in the art.

The one or more TAs may comprise a polypeptide derivative. The polypeptide derivative may comprise at least a portion of a wild-type polypeptide comprising one or more amino acid mutations. The one or more amino acid mutations may comprise a deletion, substitution, addition or a combination thereof. The one or more amino acid mutations may comprise adding one or more amino acid residues to a wild-type polypeptide. The one or more amino acid mutations may comprise deletion of one or more amino acid residues of the wild-type polypeptide. The one or more amino acid mutations may comprise substitution of one or more amino acid residues of the wild-type polypeptide. The one or more amino acid mutations may comprise substituting one or more amino acid residues of the wild-type polypeptide with one or more lysine residues. The one or more amino acid mutations may comprise substituting one or more amino acid residues of the wild-type polypeptide with one or more D-amino acid residues. The one or more amino acid residues of the wild-type polypeptide may comprise one or more alanines, methionines, arginines, serines, threonines, and tyrosines.

The one or more TAs may comprise a modified therapeutic peptide. Methods of modifying peptides are known in the art (see for example, Gentilucci L et al., 2010, Curr Pharm Des). Examples of peptide modifications include, but are not limited to, acetylation, phosphorylation, and methylation. The peptide modification may comprise a chemical modification. Peptide modifications may occur on the N-terminus of the peptide. Alternatively, or additionally, peptide modifications may occur on the C-terminus of the peptide. Peptide modifications may occur at one or more internal amino acids of the peptide. Peptide modifications may comprise replacing the carboxyl group at the C-terminus of the peptide. Peptide modifications may comprise modifying the carboxyl group at the C-terminus of the peptide. The carboxyl group at the C-terminus of the peptide may be modified to produce an amide group. The carboxyl group at the C-terminus of the peptide may be modified to produce an amine group.

The one or more staples of the PLC or mTA may be attached to two or more residues in the one or more TAs. The TA may comprise a fusion peptide. The two or more residues may be adjacent. The two or more residues may be non-adjacent. The two or more residues may be at least about 1 amino acid residue apart. The two or more residues may be at least about 2, 3, 4, 5 or more amino acid residues apart. The two or more residues may be at least about 4 amino acid residues apart. The two or more residues may be at least about 5 amino acid residues apart. The two or more residues may be at least about 6 amino acid residues apart. The two or more residues may be at least about 7 amino acid residues apart. The two or more residues may be at least about 8 amino acid residues apart. The two or more residues may be at least about 9 amino acid residues apart. The two or more residues may be at least about 10 amino acid residues apart. The two or more residues may be at least about 11 amino acid residues apart. The two or more residues may be at least about 12 amino acid residues apart. The two or more residues may be at least about 13 amino acid residues apart. The two or more residues may be at least about 14 amino acid residues apart. The two or more residues may be at least about 15 amino acid residues apart. The two or more residues may be at least about 4, 7, 11, or 14 amino acid residues apart. The two or more residues may be less than about 100 amino acid residues apart. The two or more residues may be less than about 90, 85, 80, 75, 70, 65, 60, 55 amino acid residues apart. The two or more residues may be less than about 50 amino acid residues apart. The two or more residues may be less than about 30 amino acid residues apart. The two or more residues may be less than about 20 amino acid residues apart.

The TAs may be from a mammal or non-mammal. The TAs may be from a human. Alternatively, the TAs may be from a goat, sheep, cow, rabbit, monkey, dog, cat or a combination thereof. The TAs may be from a reptile. The TAs may be from a snake or lizard. The TAs may be from an amphibian. The TAs may be from a frog or toad. The TAs may be from an avian. The TAs may be recombinant peptide.

### Linker

The PLCs or mTAs disclosed herein may further comprise one or more linkers. The PLCs or mTAs disclosed herein may further comprise two or more linkers. The PLCs or mTAs disclosed herein may further comprise three or more linkers. The PLCs or mTAs disclosed herein may further comprise four or more linkers. The PLCs or mTAs disclosed herein may further comprise five or more linkers.

The one or more linkers may enable attachment of a lipid to a peptide conjugate at a staple. The one or more linkers may enable attachment of a lipid to a staple. The linker may enable attachment of a lipid to another lipid. The linker may enable attachment of a lipid to a chemical group comprising one or more polyethylene glycol subunits. The linker may enable attachment of a PEG to another PEG. The linker may enable attachment of a PEG to a therapeutic agent. The linker may enable attachment of a therapeutic agent to another therapeutic agent. The linker may be an amino acid. The linker may be an amino acid of the therapeutic agent. The linker may be a substituted amino acid of the therapeutic agent. The linker may be a lysine. The linker may be an L-lysine. The linker may be an ether or an amide. The linker may be an amide. The linker may be a thioether. The linker may be a carbamate. The linker may be a Michael reaction adduct. The linker may link a PEG molecule to a lipid.

The linker may be formed by reaction of an amino acid on the peptide region with an electrophilic linker precursor. The linker may be formed by reaction of a lysine on the peptide region with an electrophilic linker precursor. The electrophilic linker precursor may be a staple precursor compound, a lipid staple precursor, or a lipid derivative. The linker may be formed by reaction of a derivatizable functional group on the staple precursor compound with a lipid derivative to produce a lipid staple precursor. The linker may be formed by reaction of a derivatizable functional group on the staple already attached to the therapeutic agent with a lipid derivative.

The linker may be the product of a bioorthogonal reaction. The linker may be an oxime, a tetrazole, a Diels Alder adduct, a hetero Diels Alder adduct, an aromatic substitution reaction product, a nucleophilic substitution reaction product, an ester, an amide, a carbamate, or a Michael reaction product. The linker may be a metathesis reaction product, a metal-mediated cross-coupling reaction product, a radical polymerization product, an oxidative coupling product, an acyl-transfer reaction product, or a photo click reaction product.

### Pharmacokinetics

Mechanisms by which the PLCs or mTAs positively influence pharmacokinetic or pharmacodynamic behavior include, but are not limited to, (i) preventing or mitigating in vivo proteolytic degradation or other activity-diminishing chemical modification of the therapeutic agent; (ii) improving half-life or other pharmacokinetic properties by reducing renal filtration, decreasing receptor-mediated clearance or increasing bioavailability; (iii) reducing toxicity; (iv) improving solubility; and/or (v) increasing biological activity and/or target selectivity of the therapeutic agent or unmodified therapeutic peptide.

The PLCs or mTAs may enhance one or more pharmacokinetic properties of a therapeutic agent (TA) when attached to the TA. The PLCs or mTAs disclosed herein may enhance the one or more pharmacokinetic properties of the TA by at least about 200% as measured by pharmacodynamics when compared to the TA or unmodified therapeutic peptide alone. The PLCs or mTAs disclosed herein may enhance the one or more pharmacokinetic properties of the TA by at least about 300%, 400%, 500%, 600%, 700%, 800%, 900%, 1000% as measured by pharmacodynamics when compared to the TA or unmodified therapeutic peptide alone.

The pharmacokinetic properties may comprise a half-life. The half-life of the PLC or mTA may be at least about two-fold longer compared to the half-life of the TA or unmodified therapeutic peptide alone. The half-life of the PLC or mTA disclosed herein may be at least about 3-fold, 4-fold, or 5-fold longer compared to the half-life of the TA or unmodified therapeutic peptide alone. The half-life of the PLC or mTA disclosed herein may be at least about 6-, 7-, 8-, 9-, 10-, 15-, 20-, 25-, 30-, 35-, 40-, 45-, or 50-fold longer compared to the half-life of the TA or unmodified therapeutic peptide alone.

In addition, the PLCs or mTAs may have positive effects on terms of increasing manufacturability, and/or reducing immunogenicity of the therapeutic agent, compared to an unconjugated form of the therapeutic agent or unmodified therapeutic peptide.

### Therapeutic Use

Further disclosed herein are PLCs or mTAs for treating, alleviating, inhibiting and/or preventing one or more diseases and/or conditions. The disease and/or condition may be a chronic disease or condition. Alternatively, the disease and/or condition is an acute disease or condition. The disease or condition may be recurrent, refractory, accelerated, or in remission. The disease or condition may affect one or more cell types. The one or more diseases and/or conditions may be an autoimmune disease, inflammatory disease, or metabolic disease.

Disclosed herein are methods for treating a disease or condition in a subject in need thereof, the method comprising administering to the subject one or more PLCs, wherein the one or more peptide lipid conjugates (PLCs) comprise (a) one or more lipids; and (b) one or more peptide conjugates (PC), the peptide conjugate (PC) comprising a peptide region comprising one or more peptide therapeutic agents (TA) and a staple region comprising one or more staples, the one or more staples connect two or more residues in the peptide region by the formation of an amide with an amine-containing sidechain from each of the two or more residues, wherein the one or more lipids are attached to the one or more staples. Each of the two or more residues in the peptide region independently may comprise lysine, ornithine, diaminobutyric acid, diaminopropionic acid, or homolysine. The one or more TAs may comprise a GLP-1, glucagon, oxyntomodulin, exendin-4, GLP-2, GIP, GLP-1R/GCGR dual agonist, GLP-1R/GIPR dual agonist, or GLP-1R/GCGR/GIPR tri-agonist, or a derivative thereof. The disease or condition may be a diabetes or obesity, or a medical condition associated with diabetes or obesity. The disease or condition may be non-alcoholic fatty liver disease (NAFLD), nonalcoholic steatohepatitis (NASH), or cardiovascular disease. The disease or condition may be an autoimmune disorder. The disease or condition may be Crohn's disease or ulcerative colitis. The disease or condition may be short bowel syndrome (SBS). The disease or condition may be inflammatory bowel disease (IBD), inflammatory bowel syndrome (IBS), or psoriasis. The disease or condition may be Alzheimer's disease, Parkinson's disease or Huntington's disease. The PLC may be administered with one or more additional therapeutic agents. The additional therapeutic agents may comprise one or more other diabetes drugs, DPP4 inhibitors, SGLT2 inhibitors, hypoglycemic drugs and biguanidine drugs, insulin secretogogues and sulfonyl urea drugs, TZD drugs, insulin and insulin analogs, FGF21 and analogs, leptin or leptin analogs, amylin and amylin analogs, an anti-inflammatory drug, cyclosporine A or FK506, 5-ASA, or a statin, or any combination thereof. The additional therapeutic agent may be aspirin.

Further disclosed herein are methods for treating a disease or condition in a subject in need thereof, the method comprising administering to the subject one or more PLCs, wherein the one or more peptide lipid conjugates (PLCs) comprise (a) one or more lipids, the lipids selected from a group consisting of sterols, sterol derivatives, bile acids, vitamin E derivatives, fatty di-acids, fatty acids, fatty amides, and fatty alcohols; and (b) one or more peptide conjugates (PC), the peptide conjugate comprising a peptide region comprising one or more peptide therapeutic agents (TA) and a staple region comprising one or more staples, the one or more staples connect two or more residues in the peptide region by the formation of an amide with an amine-containing sidechain from each of the two or more residues, wherein the one or more lipids are attached to the one or more staples. Each of the two or more residues in the peptide region independently may comprise lysine, ornithine, diaminobutyric acid, diaminopropionic acid, or homolysine. The one or more TAs may comprise GLP-1, glucagon, oxyntomodulin, exendin-4, GLP-2, GIP, GLP-1R/GCGR dual agonist, GLP-1R/GIPR dual agonist, or GLP-1R/GCGR/GIPR tri-agonist, or a derivative thereof. The disease or condition may be a diabetes or obesity, or a medical condition associated with diabetes or obesity. The disease or condition may be non-alcoholic fatty liver disease (NAFLD), nonalcoholic steatohepatitis (NASH), or cardiovascular disease. The disease or condition may be an autoimmune disorder. The disease or condition may be Crohn's disease or ulcerative colitis. The disease or condition may be short bowel syndrome (SBS). The disease or condition may be inflammatory bowel disease (IBD), inflammatory bowel syndrome (IBS), or psoriasis. The disease or condition may be Alzheimer's disease, Parkinson's disease or Huntington's disease. The PLC may be administered with one or more additional therapeutic agents. The additional therapeutic agents may comprise one or more other diabetes drugs, DPP4 inhibitors, SGLT2 inhibitors, hypoglycemic drugs and biguanidine drugs, insulin secretogogues and sulfonyl urea drugs, TZD drugs, insulin and insulin analogs, FGF21 and analogs, leptin or leptin analogs, amylin and amylin analogs, an anti-inflammatory drug, cyclosporine A or FK506, 5-ASA, or a statin, or any combination thereof. The additional therapeutic agent may be aspirin. The additional therapeutic agent may be aspirin.

Further disclosed herein are methods of treating a disease or condition in a subject in need thereof, the method comprising administering to the subject one or more peptide lipid conjugates (PLCs) of Formula (I). At least one of the two amino acid residues may comprise a lysine, ornithine, diaminobutyric acid, diaminopropionic acid, or homolysine. Each of the two amino acid residues in the peptide region independently may comprise lysine, ornithine, diaminobutyric acid, diaminopropionic acid, or homolysine. In some instances, at least one of the two amino acid residues is lysine. In some instances, at least two of the two amino acid residues are lysine. The one or more TAs may comprise a GLP-1, glucagon, oxyntomodulin, exendin-4, GLP-2, GIP, GLP-1R/GCGR dual agonist, GLP-1R/GIPR dual agonist, or GLP-1R/GCGR/GIPR tri-agonist, or a derivative thereof. The disease or condition may be a diabetes or obesity, or a medical condition associated with diabetes or obesity. The disease or condition may be non-alcoholic fatty liver disease (NAFLD), nonalcoholic steatohepatitis (NASH), or cardiovascular disease. The disease or condition may be an autoimmune disorder. The disease or condition may be Crohn's disease or ulcerative colitis. The disease or condition may be short bowel syndrome (SBS). The disease or condition may be inflammatory bowel disease (IBD), inflammatory bowel syndrome (IBS), or psoriasis. The disease or condition may be Alzheimer's disease, Parkinson's disease or Huntington's disease. The PLC may be administered with one or more additional therapeutic agents. The additional therapeutic agents may comprise one or more other diabetes drugs, DPP4 inhibitors, SGLT2 inhibitors, hypoglycemic drugs and biguanidine drugs, insulin secretogogues and sulfonyl urea drugs, TZD drugs, insulin and insulin analogs, FGF21 and analogs, leptin or leptin analogs, amylin and amylin analogs, an anti-inflammatory drug, cyclosporine A or FK506, 5-ASA, or a statin, or any combination thereof. The additional therapeutic agent may be aspirin.

Provided herein is a method of preventing or treating a metabolic disease or condition in a subject in need thereof comprising administering to the subject one or more PLCs, wherein the one or more peptide lipid conjugates (PLCs) comprise (a) one or more lipids; and (b) one or more peptide conjugates (PC), the peptide conjugate (PC) comprising a peptide region comprising one or more peptide therapeutic agents (TA) and a staple region comprising one or more staples, the one or more staples connect two or more residues in the peptide region by the formation of an amide with an amine-containing sidechain from each of the two or more residues, wherein the one or more lipids are attached to the one or more staples. At least one of the two or more residues may comprise a lysine, ornithine, diaminobutyric acid, diaminopropionic acid, or homolysine. The two or more residues in the peptide region may comprise lysine. In some instances, at least one of the two or more residues is lysine. In some instances, at least two of the two or more residues are lysine. The one or more TAs may comprise GLP-1, GLP-2, Exendin-4, oxyntomodulin, glucagon, FGF21, GLP-1R/GIPR dual agonist, a GLP-1R/GCGR dual agonist, or derivative thereof. The one or more TAs may comprise GLP-1, GLP-2, Exendin-4, oxyntomodulin, glucagon, FGF21, GIP, GLP-1R/GIPR dual agonist, a GLP-1R/GCGR dual agonist, or derivative thereof. The GLP-1 may be a human GLP-1. The FGF21 may be a human FGF21. The one or more lipids may comprise one or more sterols, sterol derivatives, bile acids, vitamin E derivatives, fatty di-acids, fatty acids, fatty amides, and fatty alcohols. The metabolic disease or condition may be diabetes. The metabolic disease or condition may be obesity. The metabolic disease or condition may be glycogen storage disease, phenylketonuria, maple syrup urine disease, glutaric acidemia type 1, Carbamoyl phosphate synthetase I deficiency, alcaptonuria, Medium-chain acyl-coenzyme A dehydrogenase deficiency (MCADD), acute intermittent porphyria, Lesch-Nyhan syndrome, lipoid congenital adrenal hyperplasia, congenital adrenal hyperplasia, Kearns-Sayre syndrome, Zellweger syndrome, Gaucher's disease, or Niemann Pick disease.

Provided herein is a method of preventing or treating a metabolic disease or condition in a subject in need thereof comprising administering to the subject one or more peptide lipid conjugates (PLCs) of Formula (I). At least one of the two amino acid residues may comprise a lysine, ornithine, diaminobutyric acid, diaminopropionic acid, or homolysine. In some instances, at least one of the two amino acid residues is lysine. In some instances, at least two of the two amino acid residues are lysine. The one or more TAs may comprise GLP-1, GLP-2, Exendin-4, oxyntomodulin, glucagon, FGF21, a GLP-1R/GIPR dual agonist, a GLP-1R/GCGR dual agonist, or derivative thereof. The one or more TAs may comprise GLP-1, GLP-2, Exendin-4, oxyntomodulin, glucagon, FGF21, GIP, a GLP-1R/GIPR dual agonist, a GLP-1R/GCGR dual agonist, or derivative thereof. The GLP-1 may be a human GLP-1. The FGF21 may be a human FGF21. The one or more lipids may comprise one or more sterols, sterol derivatives, bile acids, vitamin E derivatives, fatty di-acids, fatty acids, fatty amides, and fatty alcohols. The metabolic disease or condition may be diabetes. The metabolic disease or condition may be obesity. The metabolic disease or condition may be glycogen storage disease, phenylketonuria, maple syrup urine disease, glutaric acidemia type 1, Carbamoyl phosphate synthetase I deficiency, alcaptonuria, Medium-chain acyl-coenzyme A dehydrogenase deficiency (MCADD), acute intermittent porphyria, Lesch-Nyhan syndrome, lipoid congenital adrenal hyperplasia, congenital adrenal hyperplasia, Kearns-Sayre syndrome, Zellweger syndrome, Gaucher's disease, or Niemann Pick disease.

Provided herein is a method of preventing or treating NAFLD, NASH, or cardiovascular disease in a subject in need thereof comprising administering to the subject one or more PLCs, wherein the one or more peptide lipid conjugates (PLCs) comprise (a) one or more lipids; and (b) one or more peptide conjugates (PC), the peptide conjugate (PC) comprising a peptide region comprising one or more peptide therapeutic agents (TA) and a staple region comprising one or more staples, the one or more staples connect two or more residues in the peptide region by the formation of an amide with an amine-containing sidechain from each of the two or more residues, wherein the one or more lipids are attached to the one or more staples. At least one of the two or more residues may comprise a lysine, ornithine, diaminobutyric acid, diaminopropionic acid, or homolysine. The two or more residues in the peptide region may comprise lysine. In some instances, at least one of the two or more residues is lysine. In some instances, at least two of the two or more residues are lysine. The one or more lipids may comprise one or more sterols, sterol derivatives, bile acids, vitamin E derivatives, fatty di-acids, fatty acids, fatty amides, and fatty alcohols. The one or more TAs may comprise GLP-1, GLP-2, Exendin-4, oxyntomodulin, glucagon, a GLP-1R/GIPR dual agonist, a GLP-1R/GCGR dual agonist, or derivative thereof. The one or more TAs may comprise GLP-1, GLP-2, Exendin-4, oxyntomodulin, glucagon, GIP, a GLP-1R/GIPR dual agonist, a GLP-1R/GCGR dual agonist, or derivative thereof. The GLP-1 may be a human GLP-1.

Provided herein is a method of preventing or treating NAFLD, NASH, or cardiovascular disease in a subject in need thereof comprising administering to the subject one or more peptide lipid conjugates (PLCs) of Formula (I). At least one of the two amino acid residues may comprise a lysine, ornithine, diaminobutyric acid, diaminopropionic acid, or homolysine. The two amino acid residues in the peptide region may comprise lysine. In some instances, at least one of the two amino acid residues is lysine. In some instances, at least two of the two or more residues are lysine. The one or more lipids may comprise one or more sterols, sterol derivatives, bile acids, vitamin E derivatives, fatty di-acids, fatty acids, fatty amides, and fatty alcohols. The one or more lipids may comprise one or more sterols, sterol derivatives, bile acids, vitamin E derivatives, fatty di-acids, fatty acids, fatty amides, and fatty alcohols. The one or more TAs may comprise GLP-1, GLP-2, Exendin-4, oxyntomodulin, glucagon, a GLP-1R/GIPR dual agonist, a GLP-1R/GCGR dual agonist, or derivative thereof. The one or more TAs may comprise GLP-1, GLP-2, Exendin-4, oxyntomodulin, glucagon, GIP, a GLP-1R/GIPR dual agonist, a GLP-1R/GCGR dual agonist, or derivative thereof. The GLP-1 may be a human GLP-1.

Provided herein is a method of preventing or treating a disease or condition which benefits from a GLP-1R and/or glucagon receptor (GCGR) agonist in a subject in need thereof comprising administering to the subject one or more PLCs, wherein the one or more peptide lipid conjugates (PLCs) comprise (a) one or more lipids; and (b) one or more peptide conjugates (PC), the peptide conjugate (PC) comprising a peptide region comprising one or more peptide therapeutic agents (TA) and a staple region comprising one or more staples, the one or more staples connect two or more residues in the peptide region by the formation of an amide with an amine-containing sidechain from each of the two or more residues, wherein the one or more lipids are attached to the one or more staples. At least one of the two or more residues may comprise a lysine, ornithine, diaminobutyric acid, diaminopropionic acid, or homolysine. In some instances, at least one of the two or more residues is lysine. In some instances, at least two of the two or more residues are lysine. The one or more lipids may comprise one or more sterols, sterol derivatives, bile acids, vitamin E derivatives, fatty di-acids, fatty acids, fatty amides, and fatty alcohols. The one or more TAs may comprise GLP-1, GLP-2, Exendin-4, oxyntomodulin, glucagon, a GLP-1R/GIPR dual agonist, a GLP-1R/GCGR dual agonist, or derivative thereof. The one or more TAs may comprise GLP-1, GLP-2, Exendin-4, oxyntomodulin, glucagon, GIP, a GLP-1R/GIPR dual agonist, a GLP-1R/GCGR dual agonist, or derivative thereof. The GLP-1 may be a human GLP-1. The disease or condition may be a metabolic disease or disorder. The disease or condition may be diabetes. The disease or condition may be obesity. Additional diseases and/or conditions which benefit from a GLP-1R and/or GCGR agonist include, but are not limited to, dyslipidemia, cardiovascular and fatty liver diseases.

Provided herein is a method of preventing or treating a disease or condition which benefits from a GLP-1R and/or glucagon receptor (GCGR) agonist in a subject in need thereof comprising administering to the subject one or more peptide lipid conjugates (PLCs) of Formula (I). At least one of the two amino acid residues may comprise a lysine, ornithine, diaminobutyric acid, diaminopropionic acid, or homolysine. In some instances, at least one of the two amino acid residues is lysine. In some instances, at least two of the two amino acid residues are lysine. The one or more lipids may comprise one or more sterols, sterol derivatives, bile acids, vitamin E derivatives, fatty di-acids, fatty acids, fatty amides, and fatty alcohols. The one or more lipids may comprise one or more sterols, sterol derivatives, bile acids, vitamin E derivatives, fatty di-acids, fatty acids, fatty amides, and fatty alcohols. The one or more TAs may comprise GLP-1, GLP-2, Exendin-4, oxyntomodulin, glucagon, GLP-1R/GIPR dual agonist, GLP-1R/GCGR dual agonist, or derivative thereof. The one or more TAs may comprise GLP-1, GLP-2, Exendin-4, oxyntomodulin, glucagon, GIP, GLP-1R/GIPR dual agonist, GLP-1R/GCGR dual agonist, or derivative thereof. The GLP-1 may be a human GLP-1. The disease or condition may be a metabolic disease or disorder. The disease or condition may be diabetes. The disease or condition may be obesity. Additional diseases and/or conditions which benefit from a GLP-1R and/or GCGR agonist include, but are not limited to, dyslipidemia, cardiovascular and fatty liver diseases.

Further disclosed herein are methods of treating a disease or condition in a subject in need thereof, the method comprising administering to the subject one or more mTAs, wherein each of the one or more mTAs comprises a therapeutic agent (TA), a first staple, and a first half-life extending molecule (HEM); wherein the therapeutic agent is a modified or unmodified therapeutic peptide that is covalently attached to the first staple via two amino acid residues on the modified or unmodified therapeutic peptide, each of the two amino acid residues has an amine-containing sidechain for attachment to the first staple through the formation of an amide, and the first HEM is covalently attached to the first staple. The disease or condition may be a diabetes or obesity, or a medical condition associated with diabetes or obesity. The disease or condition may be non-alcoholic fatty liver disease (NAFLD), nonalcoholic steatohepatitis (NASH), or cardiovascular disease. The disease or condition may be short bowel syndrome (SBS). The disease or condition may be inflammatory bowel disease (IBD), inflammatory bowel syndrome (IBS), or psoriasis. The disease or condition may be Crohn's disease or ulcerative colitis. The disease or condition may be Alzheimer's disease, Parkinson's disease or Huntington's disease. The PLC may be administered with one or more additional therapeutic agents. The additional therapeutic agents may comprise one or more other diabetes drugs, DPP4 inhibitors, SGLT2 inhibitors, hypoglycemic drugs and biguanidine drugs, insulin secretogogues and sulfonyl urea drugs, TZD drugs, insulin and insulin analogs, FGF21 and analogs, leptin or leptin analogs, amylin and amylin analogs, an anti-inflammatory drug, cyclosporine A or FK506, 5-ASA, or a statin, or any combination thereof. The additional therapeutic agent may be aspirin.

Provided herein is a method of preventing or treating a metabolic disease or condition in a subject in need thereof comprising administering to the subject one or more mTAs, wherein each of the one or more mTAs comprises a therapeutic agent (TA), a first staple, and a first half-life extending molecule (HEM); wherein the therapeutic agent is a modified or unmodified therapeutic peptide that is covalently attached to the first staple via two amino acid residues on the modified or unmodified therapeutic peptide, each of the two amino acid residues has an amine-containing sidechain for attachment to the first staple through the formation of an amide, and the first HEM is covalently attached to the first staple. The metabolic disease or condition may be diabetes. The metabolic disease or condition may be obesity. The metabolic disease or condition may be glycogen storage disease, phenylketonuria, maple syrup urine disease, glutaric acidemia type 1, Carbamoyl phosphate synthetase I deficiency, alcaptonuria, Medium-chain acyl-coenzyme A dehydrogenase deficiency (MCADD), acute intermittent porphyria, Lesch-Nyhan syndrome, lipoid congenital adrenal hyperplasia, congenital adrenal hyperplasia, Kearns-Sayre syndrome, Zellweger syndrome, Gaucher's disease, or Niemann Pick disease.

Provided herein is a method of preventing or treating NAFLD, NASH, or cardiovascular disease in a subject in need thereof comprising administering to the subject one or more mTAs, wherein each of the one or more mTAs comprises a therapeutic agent (TA), a first staple, and a first half-life extending molecule (HEM); wherein the therapeutic agent is a modified or unmodified therapeutic peptide that is covalently attached to the first staple via two amino acid residues on the modified or unmodified therapeutic peptide, each of the two amino acid residues has an amine-containing sidechain for attachment to the first staple through the formation of an amide, and the first HEM is covalently attached to the first staple.

Provided herein is a method of preventing or treating short bowel syndrome (SBS) in a subject in need thereof comprising administering to the subject one or more mTAs, wherein each of the one or more mTAs comprises a therapeutic agent (TA), a first staple, and a first half-life extending molecule (HEM); wherein the therapeutic agent is a modified or unmodified therapeutic peptide that is covalently attached to the first staple via two amino acid residues on the modified or unmodified therapeutic peptide, each of the two amino acid residues has an amine-containing sidechain for attachment to the first staple through the formation of an amide, and the first HEM is covalently attached to the first staple.

Provided herein is a method of preventing or treating inflammatory bowel disease (IBD), inflammatory bowel syndrome (IBS), or psoriasis in a subject in need thereof comprising administering to the subject one or more mTAs, wherein each of the one or more mTAs comprises a therapeutic agent (TA), a first staple, and a first half-life extending molecule (HEM); wherein the therapeutic agent is a modified or unmodified therapeutic peptide that is covalently attached to the first staple via two amino acid residues on the modified or unmodified therapeutic peptide, each of the two amino acid residues has an amine-containing sidechain for attachment to the first staple through the formation of an amide, and the first HEM is covalently attached to the first staple.

Provided herein is a method of preventing or treating Crohn's disease or ulcerative colitis in a subject in need thereof comprising administering to the subject one or more mTAs, wherein each of the one or more mTAs comprises a therapeutic agent (TA), a first staple, and a first half-life extending molecule (HEM); wherein the therapeutic agent is a modified or unmodified therapeutic peptide that is covalently attached to the first staple via two amino acid residues on the modified or unmodified therapeutic peptide, each of the two amino acid residues has an amine-containing sidechain for attachment to the first staple through the formation of an amide, and the first HEM is covalently attached to the first staple.

Provided herein is a method of preventing or treating Alzheimer's disease, Parkinson's disease or Huntington's disease in a subject in need thereof comprising administering to the subject one or more mTAs, wherein each of the one or more mTAs comprises a therapeutic agent (TA), a first staple, and a first half-life extending molecule (HEM); wherein the therapeutic agent is a modified or unmodified therapeutic peptide that is covalently attached to the first staple via two amino acid residues on the modified or unmodified therapeutic peptide, each of the two amino acid residues has an amine-containing sidechain for attachment to the first staple through the formation of an amide, and the first HEM is covalently attached to the first staple.

Provided herein is a method of preventing or treating a disease or condition which benefits from a GLP-1R and/or glucagon receptor (GCGR) agonist in a subject in need thereof comprising administering to the subject one or more mTAs, wherein each of the one or more mTAs comprises a therapeutic agent (TA), a first staple, and a first half-life extending molecule (HEM); wherein the therapeutic agent is a modified or unmodified therapeutic peptide that is covalently attached to the first staple via two amino acid residues on the modified or unmodified therapeutic peptide, each of the two amino acid residues has an amine-containing sidechain for attachment to the first staple through the formation of an amide, and the first HEM is covalently attached to the first staple. The disease or condition may be a metabolic disease or disorder. The disease or condition may be diabetes. The disease or condition may be obesity. Additional diseases and/or conditions which benefit from a GLP-1R and/or GCGR agonist include, but are not limited to, dyslipidemia, cardiovascular and fatty liver diseases.

### Compositions

Disclosed herein are pharmaceutical compositions comprising a PLC disclosed herein. The compositions may comprise 1, 2, 3, 4, 5, 6, 7, 8, 9, 10 or more PLCs. The PLCs may be different. Alternatively, the PLCs may be the same or similar. The compositions may comprise 1, 2, 3, 4, 5, 6, 7, 8, 9, 10 or more PLCs. The PLCs may be different. The PLCs may comprise different therapeutic agents, different lipids, or a combination thereof. The PLCs may be the same or similar.

Disclosed herein are pharmaceutical compositions comprising an mTA disclosed herein. The compositions may comprise 1, 2, 3, 4, 5, 6, 7, 8, 9, 10 or more mTAs. The mTAs may be different. Alternatively, the mTAs may be the same or similar. The compositions may comprise 1, 2, 3, 4, 5, 6, 7, 8, 9, 10 or more mTAs. The mTAs may be different. The mTAs may comprise different therapeutic agents, different HEMs, or a combination thereof. The mTAs may be the same or similar.

Further disclosed herein are compositions comprising one or more mTAs, wherein each of the one or more mTAs comprises a therapeutic agent (TA), a staple, and a half-life extending molecule (HEM). The TA may be a modified or unmodified therapeutic peptide. The TA may be covalently attached to the staple via two amino acid residues on the modified or unmodified therapeutic peptide, each of the two amino acid residues has an amine-containing sidechain for attachment to the first staple through the formation of an amide. At least one of the two amino acid residues may comprise a lysine, ornithine, diaminobutyric acid, diaminopropionic acid, or homolysine. At least one of the two amino acid residues may comprise a lysine. The composition may further comprise one or more pharmaceutically acceptable salts, excipients or vehicles.

Further disclosed herein are compositions comprising one or more mTAs, wherein each of the one or more mTAs comprises a therapeutic agent (TA), a first staple, and a first half-life extending molecule (HEM); wherein the therapeutic agent is a modified or unmodified therapeutic peptide that is covalently attached to the first staple via two amino acid residues on the modified or unmodified therapeutic peptide, and each of the two amino acid residues has an amine-containing sidechain for attachment to the first staple through the formation of an amide. At least one of the two amino acid residues may comprise a lysine, ornithine, diaminobutyric acid, diaminopropionic acid, or homolysine. At least one of the two amino acid residues may comprise a lysine. The first HEM may be covalently attached to the first staple. The composition may further comprise one or more pharmaceutically acceptable salts, excipients or vehicles.

Further disclosed herein are compositions comprising one or more mTAs, wherein each of the one or more mTAs comprises a therapeutic agent (TA), a first staple, and a first half-life extending molecule (HEM); wherein the therapeutic agent is a modified or unmodified therapeutic peptide that is covalently attached to the first staple via two amino acid residues on the modified or unmodified therapeutic peptide, and each of the two amino acid residues has an amine-containing sidechain for attachment to the first staple through the formation of an amide, and the first HEM is covalently attached to the first staple. At least one of the two amino acid residues may comprise a lysine, ornithine, diaminobutyric acid, diaminopropionic acid, or homolysine. At least one of the two amino acid residues may comprise a lysine. The composition may further comprise one or more pharmaceutically acceptable salts, excipients or vehicles.

Further disclosed herein are compositions comprising one or more mTAs, wherein each of the one or more mTAs comprises a therapeutic agent (TA), a first staple, and a first half-life extending molecule (HEM); wherein the therapeutic agent is a modified or unmodified therapeutic peptide that is covalently attached to the first staple via two amino acid residues on the modified or unmodified therapeutic peptide; each of the two amino acid residues has an amine-containing sidechain for attachment to the first staple through the formation of an amide; the unmodified therapeutic peptide is selected from GLP-1, glucagon, oxyntomodulin, exendin-4, GLP-2, and GIP; the modified therapeutic peptide is a derivative of a peptide selected from GLP-1, glucagon, oxyntomodulin, exendin-4, GLP-2, and GIP, the derivative being a peptide comprising one or more amino acid additions, deletions, or substitutions, or a combination thereof; and the first HEM is covalently attached to the first staple. At least one of the two amino acid residues may comprise a lysine, ornithine, diaminobutyric acid, diaminopropionic acid, or homolysine. At least one of the two amino acid residues may comprise a lysine. The composition may further comprise one or more pharmaceutically acceptable salts, excipients or vehicles.

Further disclosed herein are compositions comprising one or more mTAs, wherein each of the one or more mTAs comprises a therapeutic agent (TA), a first staple, and a first half-life extending molecule (HEM); wherein the therapeutic agent is a modified or unmodified therapeutic peptide that is covalently attached to the first staple via two amino acid residues on the modified or unmodified therapeutic peptide; each of the two amino acid residues has an amine-containing sidechain for attachment to the first staple through the formation of an amide; the first HEM is covalently attached to the first staple; and the first HEM comprises a lipid, a polyglycol region, or a combination thereof. At least one of the two amino acid residues may comprise a lysine, ornithine, diaminobutyric acid, diaminopropionic acid, or homolysine. At least one of the two amino acid residues may comprise a lysine. The composition may further comprise one or more pharmaceutically acceptable salts, excipients or vehicles.

Further disclosed herein are compositions comprising one or more mTAs, wherein each of the one or more mTAs comprises a therapeutic agent (TA), a first staple, and a first half-life extending molecule (HEM); wherein the therapeutic agent is a modified or unmodified therapeutic peptide that is covalently attached to the first staple via two amino acid residues on the modified or unmodified therapeutic peptide; each of the two amino acid residues has an amine-containing sidechain for attachment to the first staple through the formation of an amide; the unmodified therapeutic peptide is selected from GLP-1, glucagon, oxyntomodulin, exendin-4, GLP-2, and GIP; the modified therapeutic peptide is a derivative of a peptide selected from GLP-1, glucagon, oxyntomodulin, exendin-4, GLP-2, and GIP, the derivative being a peptide comprising one or more amino acid additions, deletions, or substitutions, or a combination thereof; the first HEM is covalently attached to the first staple; and the first HEM comprises a lipid, a poly glycol region, or a combination thereof. At least one of the two amino acid residues may comprise a lysine, ornithine, diaminobutyric acid, diaminopropionic acid, or homolysine. At least one of the two amino acid residues may comprise a lysine. The composition may further comprise one or more pharmaceutically acceptable salts, excipients or vehicles.

Disclosed herein are compositions comprising one or more PLCs, wherein the one or more peptide lipid conjugates (PLCs) comprise (a) one or more lipids; and (b) one or more peptide conjugates (PC), the peptide conjugate (PC) comprising a peptide region comprising one or more peptide therapeutic agents (TA) and a staple region comprising one or more staples, the one or more staples connect two or more residues in the peptide region by the formation of an amide with an amine-containing sidechain from each of the two or more residues, wherein the one or more lipids are attached to the one or more staples. At least one of the two or more residues may comprise a lysine, ornithine, diaminobutyric acid, diaminopropionic acid, or homolysine. The two or more residues in the peptide region may comprise lysine. In some instances, at least one of the two or more residues is lysine. In some instances, at least two of the two or more residues are lysine. The composition may further comprise one or more pharmaceutically acceptable salts, excipients or vehicles.

Further disclosed herein are compositions comprising one or more PLCs, wherein the one or more peptide lipid conjugates (PLCs) comprise (a) one or more lipids, the lipids selected from a group consisting of sterols, sterol derivatives, bile acids, vitamin E derivatives, fatty di-acids, fatty acids, fatty amides, and fatty alcohols; and (b) one or more peptide conjugates (PC), the peptide conjugate comprising a peptide region comprising one or more peptide therapeutic agents (TA) and a staple region comprising one or more staples, the one or more staples connect two or more residues in the peptide region by the formation of an amide with an amine-containing sidechain from each of the two or more residues, wherein the one or more lipids are attached to the one or more staples. At least one of the two or more residues may comprise a lysine, ornithine, diaminobutyric acid, diaminopropionic acid, or homolysine. The two or more residues in the peptide region may comprise lysine. In some instances, at least one of the two or more residues is lysine. In some instances, at least two of the two or more residues are lysine. The composition may further comprise one or more pharmaceutically acceptable salts, excipients or vehicles.

Further disclosed herein are compositions comprising one or more PLCs, wherein the one or more peptide lipid conjugates (PLCs) comprise (a) one or more lipids; and (b) one or more peptide conjugates (PC), wherein the peptide conjugate comprising a peptide region comprising one or more peptide therapeutic agents (TA) and a staple region comprising one or more staples, the one or more staples connect two or more residues in the peptide region by the formation of an amide with an amine-containing sidechain from each of the two or more residues, the one or more peptide therapeutic agents comprising one or more oxyntomodulin, exendin-4, glucagon-like protein-1 (GLP-1), GLP-2, GIP, GLP-1R/GIPR dual agonist, a GLP-1R/GCGR dual agonist, or glucagon, wherein the one or more lipids are attached to the one or more staples. At least one of the two or more residues may comprise a lysine, ornithine, diaminobutyric acid, diaminopropionic acid, or homolysine. The two or more residues in the peptide region may comprise lysine. In some instances, at least one of the two or more residues is lysine. In some instances, at least two of the two or more residues are lysine. The composition may further comprise one or more pharmaceutically acceptable salts, excipients or vehicles.

Further disclosed herein are compositions comprising one or more peptide lipid conjugates (PLCs) of Formula (I). At least one of the two amino acid residues may comprise a lysine, ornithine, diaminobutyric acid, diaminopropionic acid, or homolysine. At least one of the two amino acid residues may comprise a lysine. The two amino acid residues in the peptide region may comprise lysine. The compositions disclosed herein may further comprise one or more pharmaceutically acceptable salts, excipients or vehicles. Pharmaceutically acceptable salts, excipients, or vehicles may include carriers, excipients, diluents, antioxidants, preservatives, coloring, flavoring and diluting agents, emulsifying agents, suspending agents, solvents, fillers, bulking agents, buffers, delivery vehicles, tonicity agents, cosolvents, wetting agents, complexing agents, buffering agents, antimicrobials, and surfactants.

Neutral buffered saline or saline mixed with serum albumin are exemplary appropriate carriers. The pharmaceutical compositions may include antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics, or polyethylene glycol (PEG). Also by way of example, suitable tonicity enhancing agents include alkali metal halides (preferably sodium or potassium chloride), mannitol, sorbitol, and the like. Suitable preservatives include benzalkonium chloride, thimerosal, phenethyl alcohol, methylparaben, propylparaben, chlorhexidine, sorbic acid and the like. Hydrogen peroxide also may be used as preservative. Suitable cosolvents include glycerin, propylene glycol, and PEG. Suitable complexing agents include caffeine, polyvinylpyrrolidone, beta-cyclodextrin or hydroxy-propyl-beta-cyclodextrin. Suitable surfactants or wetting agents include sorbitan esters, polysorbates such as polysorbate 80, tromethamine, lecithin, cholesterol, tyloxapal, and the like. The buffers may be conventional buffers such as acetate, borate, citrate, phosphate, bicarbonate, or Tris-HCl. Acetate buffer may be about pH 4-5.5, and Tris buffer can be about pH 7-8.5. Additional pharmaceutical agents are set forth in Remington's Pharmaceutical Sciences, 18th Edition, A. R. Gennaro, ed., Mack Publishing Company, 1990.

The composition may be in liquid form or in a lyophilized or freeze-dried form and may include one or more lyoprotectants, excipients, surfactants, high molecular weight structural additives and/or bulking agents (see, for example, U.S. Pat. Nos. 6,685,940, 6,566,329, and 6,372,716). In one embodiment, a lyoprotectant is included, which is a non-reducing sugar such as sucrose, lactose or trehalose. The amount of lyoprotectant generally included is such that, upon reconstitution, the resulting formulation will be isotonic, although hypertonic or slightly hypotonic formulations also may be suitable. In addition, the amount of lyoprotectant should be sufficient to prevent an unacceptable amount of degradation and/or aggregation of the protein upon lyophilization. Exemplary lyoprotectant concentrations for sugars (e.g., sucrose, lactose, trehalose) in the pre-lyophilized formulation are from about 10 mM to about 400 mM. In another embodiment, a surfactant is included, such as for example, nonionic surfactants and ionic surfactants such as polysorbates (e.g., polysorbate 20, polysorbate 80); poloxamers (e.g., poloxamer 188); poly(ethylene glycol) phenyl ethers (e.g., Triton); sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl ofeyl-taurate; and the MONAQUAT™. series (Mona Industries, Inc., Paterson, N.J.), polyethyl glycol, polypropyl glycol, and copolymers of ethylene and propylene glycol (e.g., Pluronics, PF68 etc). Exemplary amounts of surfactant that may be present in the pre-lyophilized formulation are from about 0.001-0.5%. High molecular weight structural additives (e.g., fillers, binders) may include for example, acacia, albumin, alginic acid, calcium phosphate (dibasic), cellulose, carboxymethylcellulose, carboxymethylcellulose sodium, hydroxyethylcellulose, hydroxypropylcellulose, hydroxypropylmethylcellulose, microcrystalline cellulose, dextran, dextrin, dextrates, sucrose, tylose, pregelatinized starch, calcium sulfate, amylose, glycine, bentonite, maltose, sorbitol, ethylcellulose, disodium hydrogen phosphate, disodium phosphate, disodium pyrosulfite, polyvinyl alcohol, gelatin, glucose, guar gum, liquid glucose, compressible sugar, magnesium aluminum silicate, maltodextrin, polyethylene oxide, polymethacrylates, povidone, sodium alginate, tragacanth microcrystalline cellulose, starch, and zein. Exemplary concentrations of high molecular weight structural additives are from 0.1% to 10% by weight. In other embodiments, a bulking agent (e.g., mannitol, glycine) may be included.

Compositions may be suitable for parenteral administration. Exemplary compositions are suitable for injection or infusion into an animal by any route available to the skilled worker, such as intraarticular, subcutaneous, intravenous, intramuscular, intraperitoneal, intracerebral (intraparenchymal), intracerebroventricular, intramuscular, intraocular, intraarterial, or intralesional routes. A parenteral formulation typically may be a sterile, pyrogen-free, isotonic aqueous solution, optionally containing pharmaceutically acceptable preservatives.

Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringers' dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present, such as, for example, anti-microbials, anti-oxidants, chelating agents, inert gases and the like. See generally, Remington's Pharmaceutical Science, 16th Ed., Mack Eds., 1980.

Pharmaceutical compositions described herein may be formulated for controlled or sustained delivery in a manner that provides local concentration of the product (e.g., bolus, depot effect) and/or increased stability or half-life in a particular local environment. The compositions can include the formulation of mTAs, PLCs, polypeptides, nucleic acids, or vectors disclosed herein with particulate preparations of polymeric compounds such as polylactic acid, polyglycolic acid, etc., as well as agents such as a biodegradable matrix, injectable microspheres, microcapsular particles, microcapsules, bioerodible particles beads, liposomes, and implantable delivery devices that provide for the controlled or sustained release of the active agent which then can be delivered as a depot injection. Techniques for formulating such sustained- or controlled-delivery means are known and a variety of polymers have been developed and used for the controlled release and delivery of drugs. Such polymers are typically biodegradable and biocompatible. Polymer hydrogels, including those formed by complexation of enantiomeric polymer or polypeptide segments, and hydrogels with temperature or pH sensitive properties, may be desirable for providing drug depot effect because of the mild and aqueous conditions involved in trapping bioactive protein agents (e.g., antibodies comprising an ultralong CDR3). See, for example, the description of controlled release porous polymeric microparticles for the delivery of pharmaceutical compositions in WO 93/15722.

Suitable materials for this purpose include polylactides (see, e.g., U.S. Pat. No. 3,773,919), polymers of poly-(α-hydroxy carboxylic acids), such as poly-D-(−)-3-hydroxy butyric acid (EP 133,988A), copolymers of L-glutamic acid and gamma ethyl-L-glutamate (Sidman et al., Biopolymers, 22:547-556 (1983)), poly(2-hydroxyethyl-methacrylate) (Langer et al., J. Biomed. Mater. Res., 15:167-277 (1981), and Langer, Chem. Tech., 12:98-105 (1982)), ethylene vinyl acetate, or poly-D (−)-3-hydroxybutyric acid. Other biodegradable polymers include poly(lactones), poly(acetals), poly(orthoesters), and poly(orthocarbonates). Sustained-release compositions also may include liposomes, which can be prepared by any of several methods known in the art (see, e.g., Eppstein et al., Proc. Natl. Acad. Sci. USA, 82:3688-92 (1985)). The carrier itself, or its degradation products, should be nontoxic in the target tissue and should not further aggravate the condition. This can be determined by routine screening in animal models of the target disorder or, if such models are unavailable, in normal animals.

Microencapsulation of recombinant proteins for sustained release has been performed successfully with human growth hormone (rhGH), interferon-(rhIFN-), interleukin-2, and MN rgp 120. Johnson et al., Nat. Med., 2:795-799 (1996); Yasuda, Biomed. Ther., 27:1221-1223 (1993); Hora et al., Bio/Technology. 8:755-758 (1990); Cleland, “Design and Production of Single Immunization Vaccines Using Polylactide Polyglycolide Microsphere Systems,” in Vaccine Design: The Subunit and Adjuvant Approach, (Powell and Newman, eds, Plenum Press: New York, 1995, pp. 439-462); WO 97/03692, WO 96/40072, WO 96/07399; and U.S. Pat. No. 5,654,010. The sustained-release formulations of these proteins were developed using poly-lactic-coglycolic acid (PLGA) polymer due to its biocompatibility and wide range of biodegradable properties. The degradation products of PLGA, lactic and glycolic acids can be cleared quickly within the human body. Moreover, the degradability of this polymer can be depending on its molecular weight and composition. Lewis, “Controlled release of bioactive agents from lactide/glycolide polymer,” in: M. Chasin and R. Langer (Eds.), Biodegradable Polymers as Drug Delivery Systems (Marcel Dekker: New York, 1990, pp. 1-41). Additional examples of sustained release compositions include, for example, EP 58,481A, U.S. Pat. No. 3,887,699, EP 158,277A, Canadian Patent No. 1176565, U. Sidman et al., Biopolymers 22, 547 (1983), R. Langer et al., Chem. Tech. 12, 98 (1982), Sinha et al., J. Control. Release 90, 261 (2003), Zhu et al., Nat. Biotechnol. 18, 24 (2000), and Dai et al., Colloids Surf B Biointerfaces 41, 117 (2005).

Bioadhesive polymers are also contemplated for use in or with compositions of the present disclosure. Bioadhesives are synthetic and naturally occurring materials able to adhere to biological substrates for extended time periods. For example, Carbopol and polycarbophil are both synthetic cross-linked derivatives of poly(acrylic acid). Bioadhesive delivery systems based on naturally occurring substances include for example hyaluronic acid, also known as hyaluronan. Hyaluronic acid is a naturally occurring mucopolysaccharide consisting of residues of D-glucuronic and N-acetyl-D-glucosamine. Hyaluronic acid is found in the extracellular tissue matrix of vertebrates, including in connective tissues, as well as in synovial fluid and in the vitreous and aqueous humor of the eye. Esterified derivatives of hyaluronic acid have been used to produce microspheres for use in delivery that are biocompatible and biodegradable (see, for example, Cortivo et al., Biomaterials (1991) 12:727-730; EP 517,565; WO 96/29998; Illum et al., J. Controlled Rel. (1994) 29:133-141).

Both biodegradable and non-biodegradable polymeric matrices may be used to deliver compositions of the present disclosure, and such polymeric matrices may comprise natural or synthetic polymers. Biodegradable matrices are preferred. The period of time over which release occurs is based on selection of the polymer. Typically, release over a period ranging from between a few hours and three to twelve months is most desirable. Exemplary synthetic polymers which may be used to form the biodegradable delivery system include: polymers of lactic acid and glycolic acid, polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyanhydrides, polyurethanes and co-polymers thereof, poly(butic acid), poly(valeric acid), alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxyethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone. Exemplary natural polymers include alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The polymer optionally is in the form of a hydrogel (see, for example, WO 04/009664, WO 05/087201, Sawhney, et al., Macromolecules, 1993, 26, 581-587) that can absorb up to about 90% of its weight in water and further, optionally is cross-linked with multi-valent ions or other polymers.

Delivery systems also include non-polymer systems that are lipids including sterols such as cholesterol, cholesterol esters and fatty acids or neutral fats such as mono-di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which the product is contained in a form within a matrix such as those described in U.S. Pat. Nos. 4,452,775, 4,675,189 and 5,736,152 and (b) diffusional systems in which a product permeates at a controlled rate from a polymer such as described in U.S. Pat. Nos. 3,854,480, 5,133,974 and 5,407,686. Liposomes containing the product may be prepared by methods known methods, such as for example (DE 3,218,121; Epstein et al., Proc. Natl. Acad. Sci. USA, 82:3688-3692 (1985); Hwang et al., Proc. Natl. Acad. Sci. USA, 77:4030-4034 (1980); EP 52,322; EP 36,676; EP 88,046; EP 143,949; EP 142,641; JP 83-118008; U.S. Pat. Nos. 4,485,045 and 4,544,545; and EP 102,324).

Alternatively or additionally, the compositions may be administered locally via implantation into the affected area of a membrane, sponge, or other appropriate material on to which a PLC, mTA, nucleic acid, or vector disclosed herein has been absorbed or encapsulated. Where an implantation device is used, the device may be implanted into any suitable tissue or organ, and delivery of an antibody comprising an ultralong CDR3 antibody fragment, nucleic acid, or vector disclosed herein can be directly through the device via bolus, or via continuous administration, or via catheter using continuous infusion.

A pharmaceutical composition comprising a PLC, mTA, nucleic acid, or vector disclosed herein may be formulated for inhalation, such as for example, as a dry powder. Inhalation solutions also may be formulated in a liquefied propellant for aerosol delivery. In yet another formulation, solutions may be nebulized. Additional pharmaceutical composition for pulmonary administration include, those described, for example, in WO 94/20069, which discloses pulmonary delivery of chemically modified proteins. For pulmonary delivery, the particle size should be suitable for delivery to the distal lung. For example, the particle size may be from 1 μm to 5 μm; however, larger particles may be used, for example, if each particle is fairly porous.

Certain formulations containing antibodies comprising a PLC, mTA, nucleic acid, or vector disclosed herein may be administered orally. Formulations administered in this fashion may be formulated with or without those carriers customarily used in the compounding of solid dosage forms such as tablets and capsules. For example, a capsule can be designed to release the active portion of the formulation at the point in the gastrointestinal tract when bioavailability is maximized and pre-systemic degradation is minimized. Additional agents may be included to facilitate absorption of a selective binding agent. Diluents, flavorings, low melting point waxes, vegetable oils, lubricants, suspending agents, tablet disintegrating agents, and binders also can be employed.

Another preparation may involve an effective quantity of an antibody comprising a PLC, mTA, nucleic acid, or vector disclosed herein in a mixture with non-toxic excipients which are suitable for the manufacture of tablets. By dissolving the tablets in sterile water, or another appropriate vehicle, solutions may be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Suitable and/or preferred pharmaceutical formulations may be determined in view of the present disclosure and general knowledge of formulation technology, depending upon the intended route of administration, delivery format, and desired dosage. Regardless of the manner of administration, an effective dose may be calculated according to patient body weight, body surface area, or organ size. Further refinement of the calculations for determining the appropriate dosage for treatment involving each of the formulations described herein are routinely made in the art and is within the ambit of tasks routinely performed in the art. Appropriate dosages may be ascertained through use of appropriate dose-response data.

### Vectors, Host Cells and Recombinant Methods

A TA, as disclosed herein, may be expressed by recombinant methods. Generally, a nucleic acid encoding a TA may be isolated and inserted into a replicable vector for further cloning (amplification of the DNA) or for expression. DNA encoding the TA may be prepared by PCR amplification and sequenced using conventional procedures (e.g., by using oligonucleotide probes that are capable of binding specifically to nucleotides encoding a TA). In an exemplary embodiment, nucleic acid encoding a TA is PCR amplified, restriction enzyme digested and gel purified. The digested DNA may be inserted into a replicable vector. The replicable vector containing the digested DNA insertion may be transformed or transduced into a host cell for further cloning (amplification of the DNA) or for expression. Host cells may be prokaryotic or eukaryotic cells.

Polynucleotide sequences encoding polypeptide components of the mTAs or PLCs disclosed herein may be obtained by PCR amplification with overlapping oligonucleotide primers. Polynucleotide sequences may be isolated and sequenced from TA producing cells. Alternatively, polynucleotides may be synthesized using nucleotide synthesizer or PCR techniques. Once obtained, sequences encoding the polypeptides are inserted into a recombinant vector capable of replicating and expressing heterologous polynucleotides in prokaryotic and/or eukaryotic hosts.

In addition, phage vectors containing replicon and control sequences that are compatible with the host microorganism may be used as transforming vectors in connection with these hosts. For example, bacteriophage such as AGEM™-11 may be utilized in making a recombinant vector which can be used to transform susceptible host cells such as E. coli LE392.

TAs may be expressed in intracellularly (e.g., cytoplasm) or extracellularly (e.g., secretion). For extracellular expression, the vector may comprise a secretion signal which enables translocation of the TA to the outside of the cell.

Suitable host cells for cloning or expression of TA-encoding vectors include prokaryotic or eukaryotic cells. The host cell may be a eukaryotic. Examples of eukaryotic cells include, but are not limited to, Human Embryonic Kidney (HEK) cell, Chinese Hamster Ovary (CHO) cell, fungi, yeasts, invertebrate cells (e.g., plant cells and insect cells), lymphoid cell (e.g., YO, NSO, Sp20 cell). Other examples of suitable mammalian host cell lines are monkey kidney CV1 line transformed by SV40 (COS-7); baby hamster kidney cells (BHK); mouse Sertoli cells; monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (HepG2); mouse mammary tumor (MMT 060562); TRI cells; MRC 5 cells; and FS4 cells. The host cell may be a prokaryotic cell (e.g., E. coli).

Host cells may be transformed with vectors containing nucleotides encoding a TA. Transformed host cells may be cultured in media. The media may be supplemented with one or more agents for inducing promoters, selecting transformants, or amplifying or expressing the genes encoding the desired sequences. Methods for transforming host cells are known in the art and may include electroporation, calcium chloride, or polyethylene glycol/DMSO.

Alternatively, host cells may be transfected or transduced with vectors containing nucleotides encoding a TA. Transfected or transduced host cells may be cultured in media. The media may be supplemented with one or more agents for inducing promoters, selecting transfected or transduced cells, or expressing genes encoding the desired sequences.

The expressed TAs may be secreted into and recovered from the periplasm of the host cells or transported into the culture media. Protein recovery from the periplasm may involve disrupting the host cell. Disruption of the host cell may comprise osmotic shock, sonication or lysis. Centrifugation or filtration may be used to remove cell debris or whole cells. The TAs may be further purified, for example, by affinity resin chromatography.

Alternatively, TAs that are secreted into the culture media may be isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

TA production may be conducted in large quantity by a fermentation process. Various large-scale fed-batch fermentation procedures are available for production of recombinant proteins. Large-scale fermentations have at least 1000 liters of capacity, preferably about 1,000 to 100,000 liters of capacity. These fermentors use agitator impellers to distribute oxygen and nutrients, especially glucose (a preferred carbon/energy source). Small scale fermentation refers generally to fermentation in a fermentor that is no more than approximately 100 liters in volumetric capacity, and can range from about 1 liter to about 100 liters.

In a fermentation process, induction of protein expression is typically initiated after the cells have been grown under suitable conditions to a desired density, e.g., an OD550 of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction time may be used.

To improve the production yield and quality of the TAs disclosed herein, various fermentation conditions can be modified. For example, to improve the proper assembly and folding of the secreted TA polypeptides, additional vectors overexpressing chaperone proteins, such as Dsb proteins (DsbA, DsbB, DsbC, DsbD and or DsbG) or FkpA (a peptidylprolyl cis, trans-isomerase with chaperone activity) may be used to co-transform the host prokaryotic cells. The chaperone proteins have been demonstrated to facilitate the proper folding and solubility of heterologous proteins produced in bacterial host cells.

To minimize proteolysis of expressed heterologous proteins (especially those that are proteolytically sensitive), certain host strains deficient for proteolytic enzymes can be used for the present disclosure. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof. Some E. coli protease-deficient strains are available.

Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, hydroxylapatite chromatography, gel electrophoresis, dialysis, and affinity chromatography and gel filtration using, for example, Sephadex G-75.

TAs may be concentrated using a commercially available protein concentration filter, for example, an Amicon or Millipore Pellicon® ultrafiltration unit.

Protease inhibitors or protease inhibitor cocktails may be included in any of the foregoing steps to inhibit proteolysis of the TA.

In some cases, a TA or fragment thereof may not be biologically active upon isolation. Various methods for “refolding” or converting a polypeptide to its tertiary structure and generating disulfide linkages, can be used to restore biological activity. Such methods include exposing the solubilized polypeptide to a pH usually above 7 and in the presence of a particular concentration of a chaotrope. The selection of chaotrope is very similar to the choices used for inclusion body solubilization, but usually the chaotrope is used at a lower concentration and is not necessarily the same as chaotropes used for the solubilization. In most cases the refolding/oxidation solution will also contain a reducing agent or the reducing agent plus its oxidized form in a specific ratio to generate a particular redox potential allowing for disulfide shuffling to occur in the formation of the protein's cysteine bridge(s). Some of the commonly used redox couples include cystein/cystamine, glutathione (GSH)/dithiobis GSH, cupric chloride, dithiothreitol (DTT)/dithiane DTT, and 2-mercaptoethanol (bME)/di-thio-b (ME). In many instances, a cosolvent may be used to increase the efficiency of the refolding, and common reagents used for this purpose include glycerol, polyethylene glycol of various molecular weights, arginine and the like.

### Kits/Articles of Manufacture

As an additional aspect, the present disclosure includes kits which comprise one or more compounds or compositions packaged in a manner which facilitates their use to practice methods of the present disclosure. In one embodiment, such a kit includes a compound or composition described herein (e.g. a PLC or mTA alone or in combination with a second agent), packaged in a container with a label affixed to the container or a package insert that describes use of the compound or composition in practicing the method. Suitable containers include, for example, bottles, vials, syringes, etc. The containers may be formed from a variety of materials such as glass or plastic. The container may have a sterile access port (for example the container may be an intravenous solution bag or a vial having a stopper pierceable by a hypodermic injection needle). The article of manufacture may comprise (a) a first container with a composition contained therein, wherein the composition comprises a PLC or mTA as disclosed herein; and (b) a second container with a composition contained therein, wherein the composition comprises a further therapeutic agent. The article of manufacture in this embodiment disclosed herein may further comprise a package insert indicating that the first and second compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes. Preferably, the compound or composition is packaged in a unit dosage form. The kit may further include a device suitable for administering the composition according to a specific route of administration or for practicing a screening assay. Preferably, the kit contains a label that describes use of the PLC or mTA composition.

In certain embodiments, the composition comprising the antibody is formulated in accordance with routine procedures as a pharmaceutical composition adapted for intravenous administration to mammals, such as humans, bovines, felines, canines, and murines. Typically, compositions for intravenous administration are solutions in sterile isotonic aqueous buffer. Where necessary, the composition may also include a solubilising agent and a local anaesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The amount of the composition described herein which will be effective in the treatment, inhibition and prevention of a disease or disorder associated with aberrant expression and/or activity of a Therapeutic protein can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses are extrapolated from dose-response curves derived from in vitro or animal model test systems.

### Definitions

The terms below, as used herein, have the following meanings, unless indicated otherwise:

“Amino” refers to the —NH₂ radical.

“Hydroxy” or “hydroxyl” refers to the —OH radical.

“Nitro” refers to the —NO₂ radical.

“Oxo” refers to the ═O substituent.

“Oxime” refers to the ═N—OH substituent.

“Alkyl” refers to a straight or branched hydrocarbon chain radical, has from one to thirty carbon atoms, and is attached to the rest of the molecule by a single bond. Alkyls comprising any number of carbon atoms from 1 to 30 are included. An alkyl comprising up to 30 carbon atoms is referred to as a C₁-C₃₀ alkyl, likewise, for example, an alkyl comprising up to 12 carbon atoms is a C₁-C₁₂ alkyl. Alkyls (and other moieties defined herein) comprising other numbers of carbon atoms are represented similarly. Alkyl groups include, but are not limited to, C₁-C₃₀ alkyl, C₁-C₂₀ alkyl, C₁-C₁₅ alkyl, C₁-C₁₀ alkyl, C₁-C₈ alkyl, C₁-C₆ alkyl, C₁-C₄ alkyl, C₁-C₃ alkyl, C₁-C₂ alkyl, C₂-C₅ alkyl, C₃-C₈ alkyl and C₄-C₈ alkyl. Representative alkyl groups include, but are not limited to, methyl, ethyl, n-propyl, 1-methylethyl (iso-propyl), n-butyl, i-butyl, s-butyl, n-pentyl, 1,1-dimethylethyl (t-butyl), 3-methylhexyl, 2-methylhexyl, vinyl, allyl, propynyl, and the like. Alkyl comprising unsaturations include alkenyl and alkynyl groups. Unless stated otherwise specifically in the specification, an alkyl group may be optionally substituted as described below.

“Alkylene” or “alkylene chain” refers to a straight or branched divalent hydrocarbon chain, as described for alkyl above. Unless stated otherwise specifically in the specification, an alkylene group may be optionally substituted as described below.

“Alkoxy” refers to a radical of the formula —ORₐ where Rₐ is an alkyl radical as defined. Unless stated otherwise specifically in the specification, an alkoxy group may be optionally substituted as described below.

“Aryl” refers to a radical derived from a hydrocarbon ring system comprising hydrogen, 6 to 30 carbon atoms and at least one aromatic ring. The aryl radical may be a monocyclic, bicyclic, tricyclic or tetracyclic ring system, which may include fused or bridged ring systems. Aryl radicals include, but are not limited to, aryl radicals derived from the hydrocarbon ring systems of aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals that are optionally substituted.

“Cycloalkyl” or “carbocycle” refers to a stable, non-aromatic, monocyclic or polycyclic carbocyclic ring, which may include fused or bridged ring systems, which is saturated or unsaturated. Representative cycloalkyls or carbocycles include, but are not limited to, cycloalkyls having from three to fifteen carbon atoms, from three to ten carbon atoms, from three to eight carbon atoms, from three to six carbon atoms, from three to five carbon atoms, or three to four carbon atoms. Monocyclic cycloalkyls or carbocycles include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Unless otherwise stated specifically in the specification, a cycloalkyl or carbocycle group may be optionally substituted.

“Fused” refers to any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo, chloro, fluoro or iodo.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Haloalkoxy” similarly refers to a radical of the formula —ORₐ where Rₐ is a haloalkyl radical as defined. Unless stated otherwise specifically in the specification, a haloalkoxy group may be optionally substituted as described below.

“Heterocycloalkyl” or “heterocyclyl” or “heterocyclic ring” or “heterocycle” refers to a stable 3- to 24-membered non-aromatic ring radical comprising 2 to 23 carbon atoms and from one to 8 heteroatoms selected from the group consisting of nitrogen, oxygen, phosphorous and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 12-crown-4, 15-crown-5, 18-crown-6, 21-crown-7, aza-18-crown-6, diaza-18-crown-6, aza-21-crown-7, and diaza-21-crown-7. Unless stated otherwise specifically in the specification, a heterocyclyl group may be optionally substituted. The term heterocycloalkyl also includes all ring forms of the carbohydrates, including but not limited to the monosaccharides, the disaccharides and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

The term “heteroaryl” as used herein, alone or in combination, refers to optionally substituted aromatic monoradicals containing from about five to about twenty skeletal ring atoms, where one or more of the ring atoms is a heteroatom independently selected from among oxygen, nitrogen, sulfur, phosphorous, silicon, selenium and tin but not limited to these atoms and with the proviso that the ring of said group does not contain two adjacent O or S atoms. In embodiments in which two or more heteroatoms are present in the ring, the two or more heteroatoms can be the same as each another, or some or all of the two or more heteroatoms can each be different from the others. The term heteroaryl includes optionally substituted fused and non-fused heteroaryl radicals having at least one heteroatom. The term heteroaryl also includes fused and non-fused heteroaryls having from five to about twelve skeletal ring atoms, as well as those having from five to about ten skeletal ring atoms. Bonding to a heteroaryl group can be via a carbon atom or a heteroatom. Thus, as a non-limiting example, an imidiazole group may be attached to a parent molecule via any of its carbon atoms (imidazol-2-yl, imidazol-4-yl or imidazol-5-yl), or its nitrogen atoms (imidazol-1-yl or imidazol-3-yl). Likewise, a heteroaryl group may be further substituted via any or all of its carbon atoms, and/or any or all of its heteroatoms. A fused heteroaryl radical may contain from two to four fused rings where the ring of attachment is a heteroaromatic ring and the other individual rings may be alicyclic, heterocyclic, aromatic, heteroaromatic or any combination thereof. A non-limiting example of a single ring heteroaryl group includes pyridyl; fused ring heteroaryl groups include benzimidazolyl, quinolinyl, acridinyl; and a non-fused bi-heteroaryl group includes bipyridinyl. Further examples of heteroaryls include, without limitation, furanyl, thienyl, oxazolyl, acridinyl, azepinyl, phenazinyl, benzimidazolyl, benzindolyl, benzofuranyl, benzofuranonyl, benzoxazolyl, benzothiazolyl, benzothiadiazolyl, benzothiophenyl, benzoxadiazolyl, benzodioxolyl, benzo[b][1,4]dioxepinyl, 1,4-benzodioxanyl, benzonaphthofuranyl, benzotriazolyl, benzodioxolyl, benzodioxinyl, benzopyranyl, benzopyranonyl, benzothienyl (benzothiophenyl), benzo[4,6]imidazo[1,2-a]pyridinyl, carbazolyl, cinnolinyl, dibenzofuranyl, dibenzothiophenyl, furanonyl, imidazolyl, indolyl, isoxazolyl, isoquinolinyl, indolizinyl, indazolyl, isoindolyl, indolinyl, isoindolinyl, indolizinyl, isothiazolyl, isoindolyloxadiazolyl, indazolyl, naphthyridinyl, oxadiazolyl, 2-oxoazepinyl, oxiranyl, 1-oxidopyridinyl, 1-oxidopyrimidinyl, 1-oxidopyrazinyl, 1-oxidopyridazinyl, 1-phenyl-1H-pyrrolyl, phenothiazinyl, phenoxazinyl, pyridinyl, pyridazinyl, pyrimidinyl, pyrazinyl, pyrrolyl, pyrazinyl, pyrazolyl, purinyl, phthalazinyl, pteridinyl, quinolinyl, quinazolinyl, quinoxalinyl, quinuclidinyl, triazolyl, tetrazolyl, thiazolyl, triazinyl, thiadiazolyl, tetrahydroquinolinyl, thiazolyl, and thiophenyl and the like, and their oxides, such as for example pyridyl-N-oxide.

All the above groups may be either substituted or unsubstituted. The term “substituted” as used herein means any of the above groups (e.g., alkyl, alkylene, alkoxy, aryl, cycloalkyl, haloalkyl, heterocyclyl and/or heteroaryl) may be further functionalized wherein at least one hydrogen atom is replaced by a bond to a non-hydrogen atom substituent. Unless stated specifically in the specification, a substituted group may include one or more substituents selected from: oxo, amino, —CO₂H, nitrile, nitro, hydroxyl, thiooxy, alkyl, alkylene, alkoxy, aryl, cycloalkyl, heterocyclyl, heteroaryl, dialkylamines, arylamines, alkylarylamines, diarylamines, trialkylammonium (—N⁺R₃), N-oxides, imides, and enamines; a silicon atom in groups such as trialkylsilyl groups, dialkylarylsilyl groups, alkyldiarylsilyl groups, triarylsilyl groups, perfluoroalkyl or perfluoroalkoxy, for example, trifluoromethyl or trifluoromethoxy. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NH₂, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. In the foregoing, R_{g} and Rₕ are the same or different and independently hydrogen, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl. In addition, each of the foregoing substituents may also be optionally substituted with one or more of the above substituents. Furthermore, any of the above groups may be substituted to include one or more internal oxygen, sulfur, or nitrogen atoms. For example, an alkyl group may be substituted with one or more internal oxygen atoms to form an ether or polyether group. Similarly, an alkyl group may be substituted with one or more internal sulfur atoms to form a thio ether, disulfide, etc.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons.

A “tautomer” refers to a proton shift from one atom of a molecule to another atom of the same molecule. The compounds presented herein may exist as tautomers. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH. Some examples of tautomeric interconversions include:

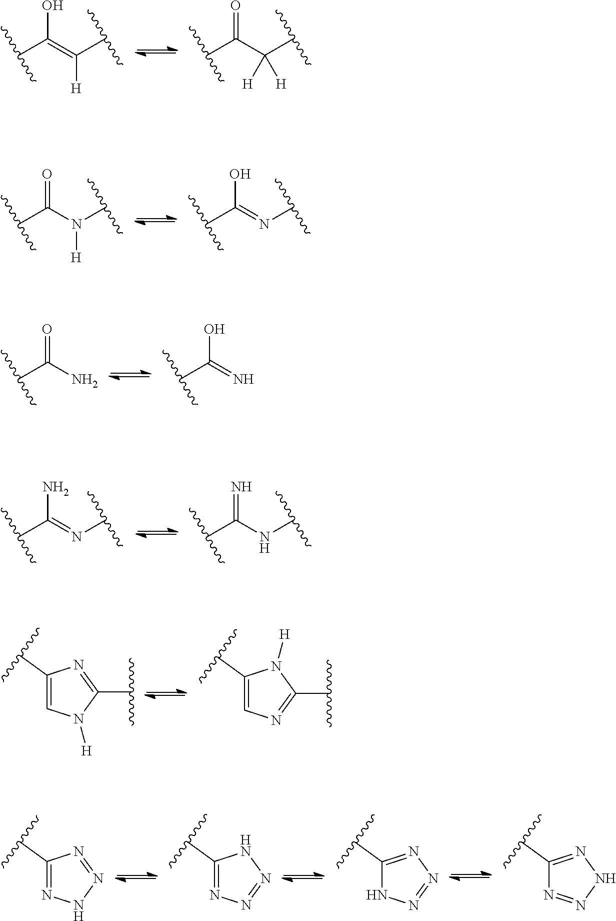

A “metabolite” of a compound disclosed herein is a derivative of that compound that is formed when the compound is metabolized. The term “active metabolite” refers to a biologically active derivative of a compound that is formed when the compound is metabo lized. The term “metabolized,” as used herein, refers to the sum of the processes (including, but not limited to, hydrolysis reactions and reactions catalyzed by enzymes, such as, oxidation reactions) by which a particular substance is changed by an organism. Thus, enzymes may produce specific structural alterations to a compound. For example, cytochrome P450 catalyzes a variety of oxidative and reductive reactions while uridine diphosphate glucuronyl transferases catalyze the transfer of an activated glucuronic-acid molecule to aromatic alcohols, aliphatic alcohols, carboxylic acids, amines and free sulfhydryl groups. Further information on metabolism may be obtained from The Pharmacological Basis of Therapeutics, 9th Edition, McGraw-Hill (1996). Metabolites of the compounds disclosed herein can be identified either by administration of compounds to a host and analysis of tissue samples from the host, or by incubation of compounds with hepatic cells in vitro and analysis of the resulting compounds. Both methods are well known in the art. Metabolites of a compound may be formed by oxidative processes and correspond to the corresponding hydroxy-containing compound. A compound may be metabolized to one or more pharmacologically active metabolites.

As used herein, a derivative of a peptide refers to, but is not limited to, a modified peptide that allows for staple and/or HEM attachment (such as one or more amino acid residue replacements or L-vs D-amino acid replacements), a fragment, an analog with one or more additional amino acids, a complex and/or an aggregate of the peptide. A derivative of a peptide may be a homolog that has at least 50% homology with respect to the peptide. A derivative of a peptide may be a homolog that has at least 60% homology with respect to the peptide. A derivative of a peptide may be a homolog that has at least 70% homology with respect to the peptide. A derivative of a peptide may be a homolog that has at least 80% homology with respect to the peptide. A derivative of a peptide may be a homolog that has at least 90% homology with respect to the peptide.

“Pharmaceutically acceptable” refers to approved or approvable by a regulatory agency of the Federal or a state government or listed in the U.S. Pharmacopeia or other generally recognized pharmacopeia for use in animals, including humans.

“Pharmaceutically acceptable salt” refers to a salt of a compound that is pharmaceutically acceptable and that possesses the desired pharmacological activity of the parent compound.

“Pharmaceutically acceptable excipient, carrier or adjuvant” refers to an excipient, carrier or adjuvant that may be administered to a subject, together with at least one antibody of the present disclosure, and which does not destroy the pharmacological activity thereof and is nontoxic when administered in doses sufficient to deliver a therapeutic amount of the compound.

“Pharmaceutically acceptable vehicle” refers to a diluent, adjuvant, excipient, or carrier with which at least one antibody of the present disclosure is administered.

Terms such as “treating” or “treatment” or “to treat” or “alleviating” or “to alleviate” may refer to: 1) therapeutic measures that cure, slow down, lessen symptoms of, and/or halt progression of a diagnosed pathologic condition or disorder; and/or 2) prophylactic or preventative measures that prevent and/or slow the development of a targeted pathologic condition or disorder. “Treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. Thus those in need of treatment may include those already with the disorder; those prone to have the disorder; and those in whom the disorder is to be prevented.

“Amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxy glutamate, and O-phosphoserine. Amino acid analogs refers to compounds that have the same basic chemical structure as a naturally occurring amino acid, e.g., an alpha carbon that is bound to a hydrogen, a carboxyl group, an amino group, and an R group, e.g., homoserine, norleucine, methionine sulfoxide, methionine methyl sulfonium. Such analogs can have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

As used herein, the term “therapeutic agent” or “peptide therapeutic agent” or “therapeutic peptide” refers to a protein or peptide that modulates the activity of another protein, peptide, cell or tissue. Modulating the activity can comprise increasing, decreasing, stimulating, or preventing the activity or expression of the protein, peptide, cell or tissue. Therapeutic agents may modulate the activity of proteins or peptides involved in the etiology of a disease or disorder. Exemplary TAs may include, but are not limited to, at least a portion of a hormone, kinase, receptor, ligand, growth factor, regulatory protein, metabolic protein, cytokine, chemokine, interferon, phosphatase, antibody or any combination thereof.

“Disorder” or “disease” refers to a condition that would benefit from treatment with a substance/molecule (e.g., an mTA or PLC as disclosed herein) or method disclosed herein. This includes chronic and acute disorders or diseases including those pathological conditions which predispose the mammal to the disorder in question.

“Mammal” for purposes of treatment refers to any animal classified as a mammal, including humans, rodents (e.g., mice and rats), and monkeys; domestic and farm animals; and zoo, sports, laboratory, or pet animals, such as dogs, cats, cattle, horses, sheep, pigs, goats, rabbits, etc. In some embodiments, the mammal is selected from a human, rodent, or monkey.

### EXAMPLES

### Example 1. Synthesis of P1, P2, and P3

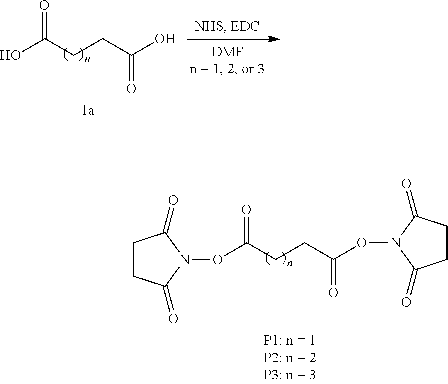

To a solution of diacid 1a (2 mmol) and hydroxysuccinimide (0.55 g, 4.8 mmol) in 30 mL of DMF was added EDC (0.75 g, 4.8 mmol) over 4 min. The reaction mixture was stirred for 22 h and the solvent was removed under reduced pressure. The residue was taken up in EtOAc (150 mL) and transferred to a separatory funnel. The organic phase was washed (1 N HCl, brine, sat. NaHCO₃, brine), dried (Na₂SO₄), filtered, and concentrated to afford the product as a white solid, which was used without additional purification or characterization.

### Example 2. Synthesis of P4

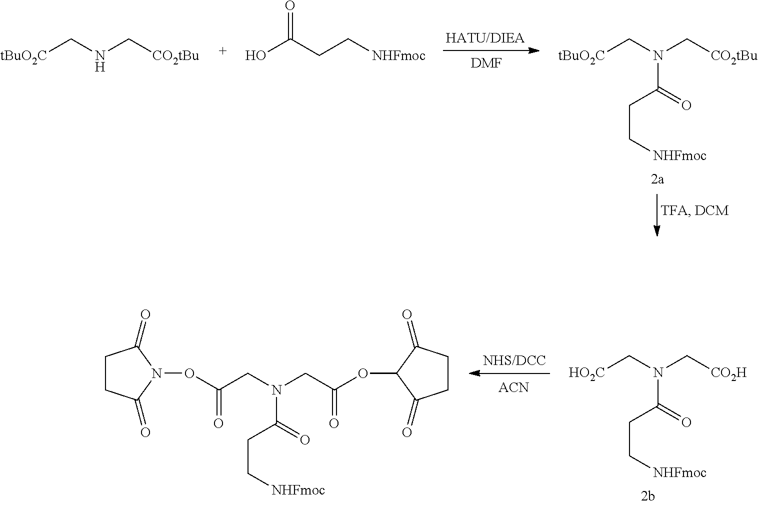

### Step 1.

To a solution of Fmoc-β-Ala-OH (3.12 g, 10 mmol), di-tert-butyl iminodiacetate (2.45 g, 10 mmol) and DIEA (1.8 mL, 10 mmol) in 100 mL of DMF at 0° C. was added HATU (3.8 g, 10 mmol) over 5 min. The reaction mixture was allowed to warm to room temperature and was stirred for 4 h. The solvent was removed under reduced pressure. The residue was taken up in EtOAc (250 mL) and transferred to a separatory funnel. The organic phase was washed (sat. NaHCO₃, brine, 1 N HCl, brine), dried (Na₂SO₄), filtered, concentrated and purified by flash column chromatography on silica gel to yield 4.7 g of intermediate 2a as a white solid (87% yield).

### Step 2.

The diester 2a (2.7 g, 5 mmol) was taken up in 15 mL of DCM at 0° C. and 30 mL of TFA was added. The reaction mixture was allowed to warm to room temperature and was stirred for 2 h. The solvent was removed under reduced pressure. The residue was washed with cold ether twice, dried to yield 3.9 g of intermediate 2b as a pale yellow foam (91% yield).

### Step 3.

To a solution of diacid 2b (0.85 g, 2 mmol) and hydroxysuccinimide (0.55 g, 4.8 mmol) in 50 mL of CH₃CN was added DCC (1.0 g, 4.8 mmol) over 5 min. A white precipitate formed immediately. The reaction mixture was stirred for 22 h and was filtered to remove the DCC urea. The solvent was removed under reduced pressure and the residue was taken up in EtOAc (150 mL) and transferred to a separatory funnel. The organic phase was washed (sat. NaHCO₃, brine, 1 N HCl, brine), dried (Na₂SO₄), filtered, and concentrated to afford a white solid. The solid was taken up in 50 mL of CH₃CN, filtered, and concentrated to yield 0.88 g of P4 as a white solid (71% yield).

### Example 3. Synthesis of mTA 4

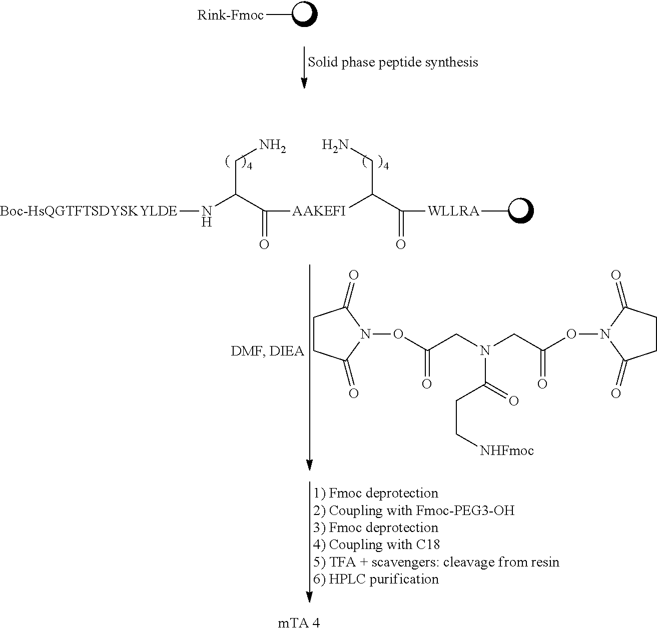

On-resin peptides were synthesized using standard fluorenylmethyloxycarbonyl (Fmoc) based solid phase peptide synthesis (SPPS) techniques using an automated peptide synthesizer.

### Peptide Cyclization Via Lactam Formation:

Pre-swollen resin containing a 29-mer peptide (0.1 mmol, 1 equiv) was added to a 25 mL round-bottomed flask. To this resin was added linker L⁴ (124 mg, 0.2 mmol, 2 equiv) dissolved in DMF (5 mL) and the flask was sparged with N₂ for 5 min. DIEA (87 μL, 0.5 mmol, 5 equiv) was added and the solution was stirred at room temperature with a stir bar for 12-16 h. The resin was filtered and washed with DMF (5×5 mL). Standard Fmoc-based solid phase peptide synthesis (SPPS) techniques were used to attach a short PEG (Fmoc-PEG3-OH) and octadecanedioic acid, then cleaved with TFA to yield the crude product. The crude peptide was dissolved in CH₃CN (5 mL), and this solution was diluted to a final volume of 20 mL with water and filtered. The filtered solution (10 mL) was loaded onto a preparative HPLC column (Phenomenex, Prep C18, 300A, 50×250 mm) equilibrated with 35% CH₃CN (0.05% TFA) in water (0.05% TFA). The composition of the eluent then was ramped to 35% CH₃CN-water (0.05% TFA) over 1 min, and a linear gradient was initiated at a rate of 1%/min of CH₃CN (0.05% TFA) into water (0.05% TFA) and run for 40 min. Eluted fractions were checked for purity on an analytical reversed phase C₁₈ column (Phenomenex, C18, 120A, 4.6×50 mm) and fractions containing the product in >95% purity were combined and freeze-dried to afford mTA4 as a white solid (11% yield).

mTA1-mTA3 and mTA5 in table 5 were synthesized as described in Example 3 using the staple precursors P1, P2, and P3. The resin cleavage step was done right after the reaction of the staple precursor with Exendin-4 (A).

mTA6 and mTA7 in table 5 and table 6 were synthesized as described in Example 3 by reacting the peptide with the P4 staple precursor and further derivatizing the compounds obtained with the appropriate acid (myristic acid or octadecanedioic acid) to arrive at the final peptide/staple/HEM.

mTA9, mTA12-mTA16 in table 7 are synthesized as described in Example 3 by reacting the peptide with the P4 staple precursor and further derivatizing the compounds obtained with the appropriate acid (myristic acid or octadecanedioic acid) to arrive at the final peptide/staple/HEM.

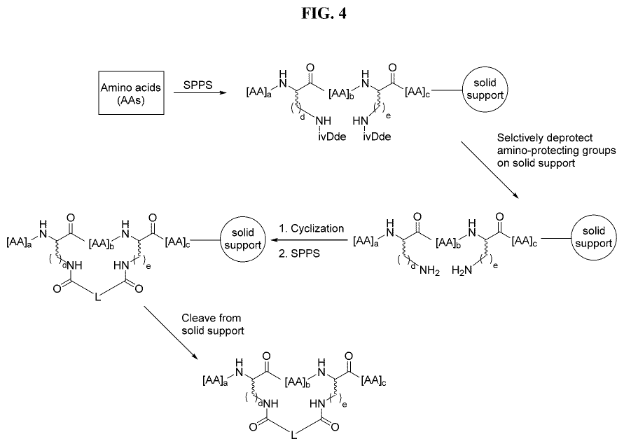

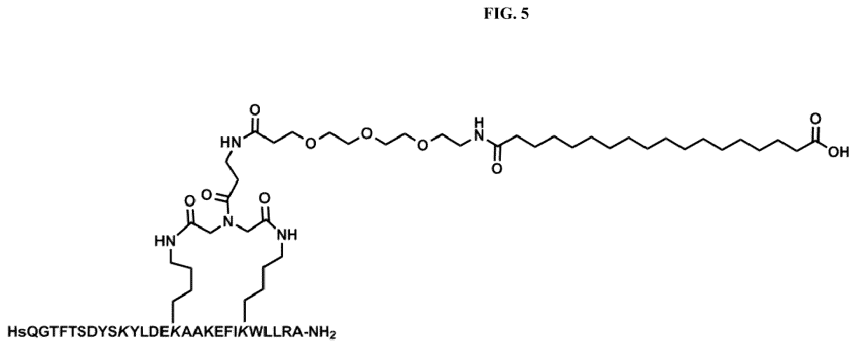

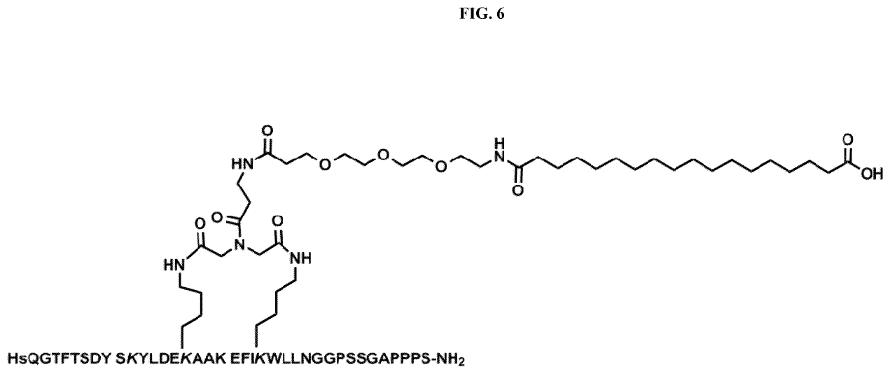

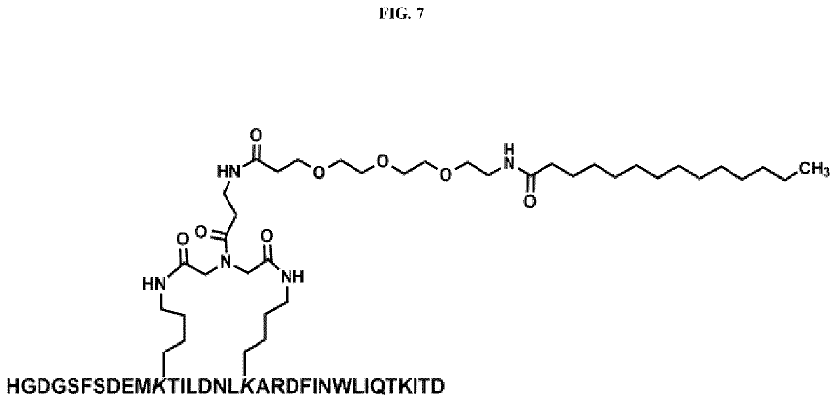

FIG. 4 depicts a general macrocyclization route to obtain the desired mTAs. The staple, with one or more attached HEMS, is depicted as —C(O)LC(O)—.

Additional compounds in Table 1 may be prepared using analogous procedures.

TABLE 1

| Entry | Structure* |
|---|---|
| 1 | 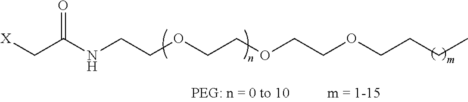 |
| 2 | 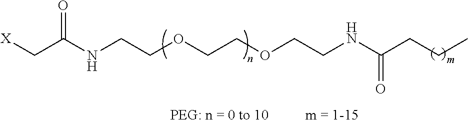 |
| 3 | 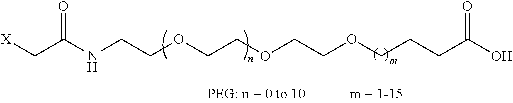 |
| 4 | 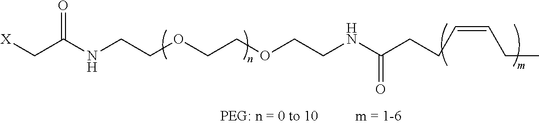 |
| 5 | 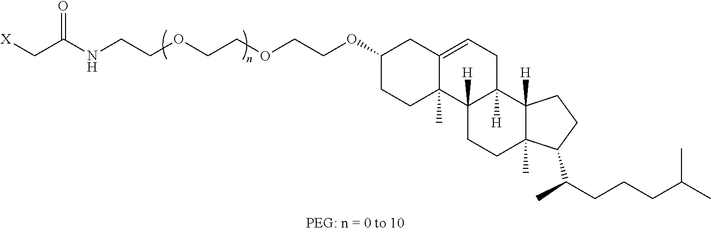 |
| 6 | 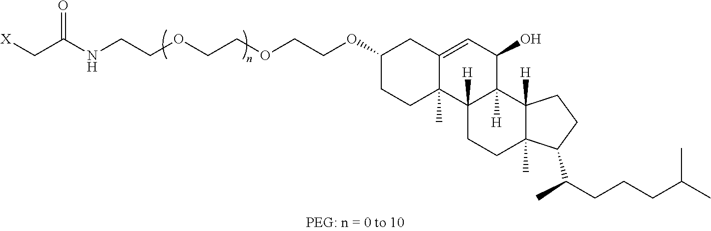 |
| 7 | 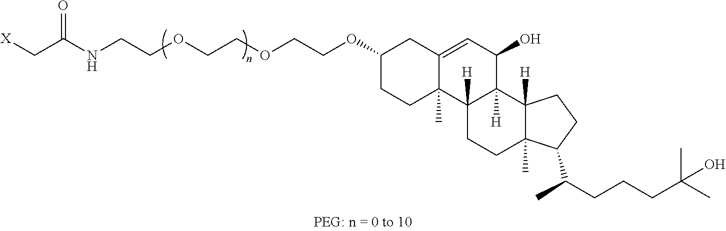 |
| 8 | 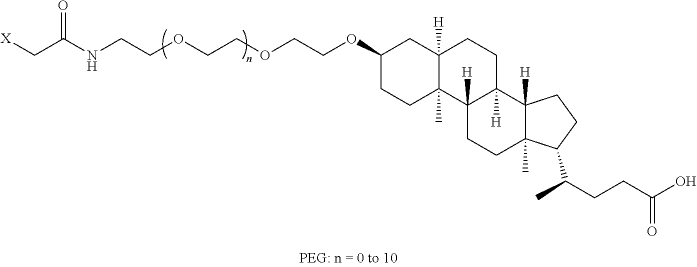 |
| 9 | 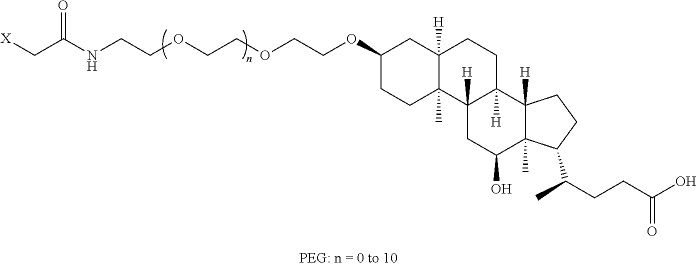 |
| 10 | 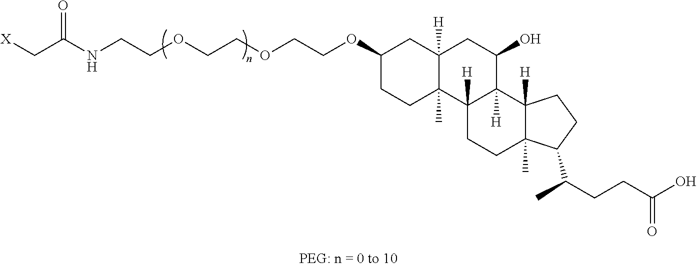 |
| 11 | 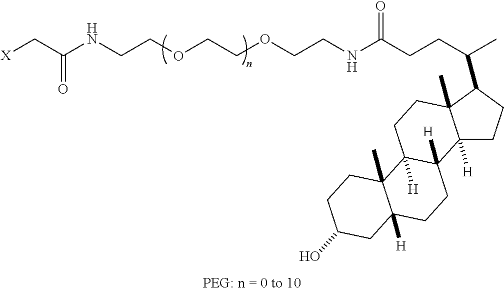 |
| 12 | 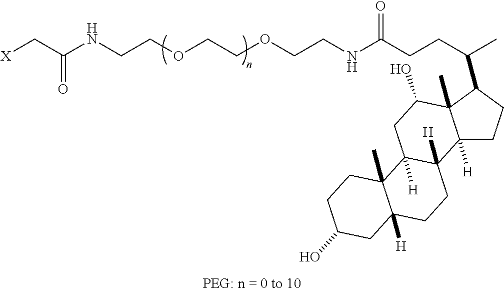 |
| 13 | 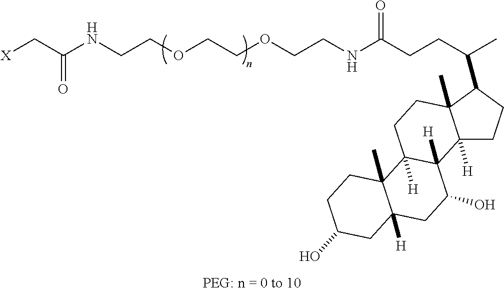 |
| 14 | 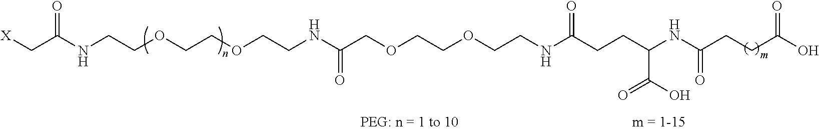 |
| 15 | 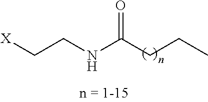 |
| 16 | 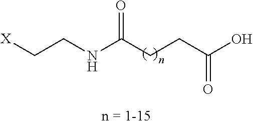 |
| 17 | 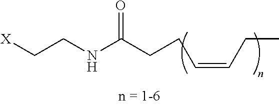 |
| 18 | 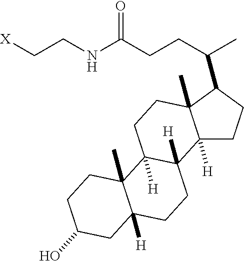 |
| 19 | 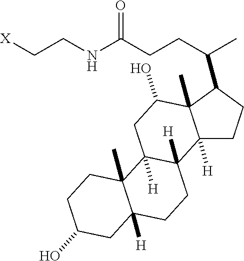 |
| 20 | 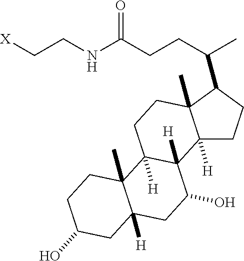 |
| 21 | 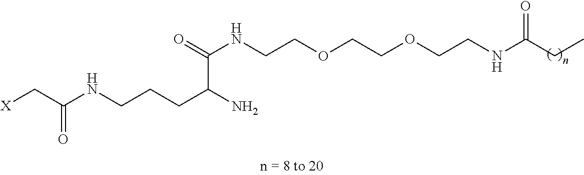 |
| 22 | 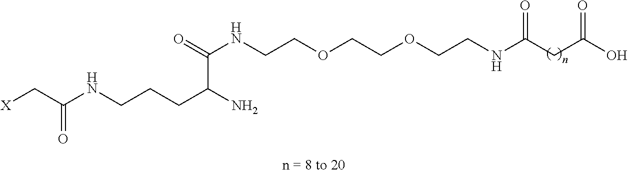 |
| 23 | 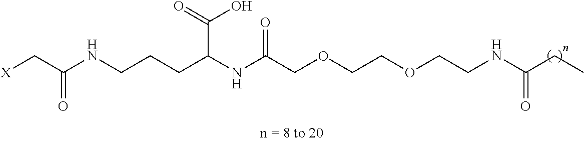 |
| 24 | 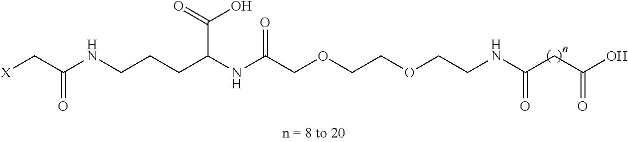 |
| 25 | 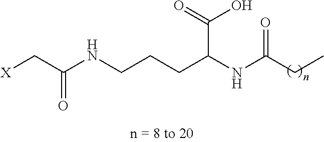 |
| 26 | 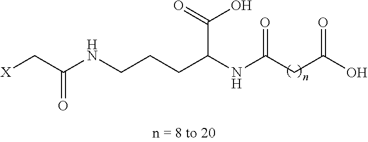 |
| 27 | 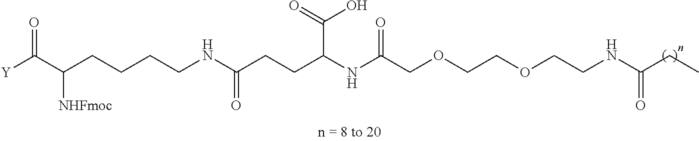 |
| 28 | 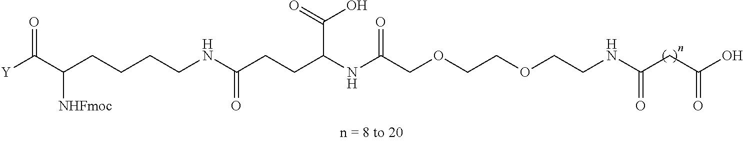 |
| 29 | 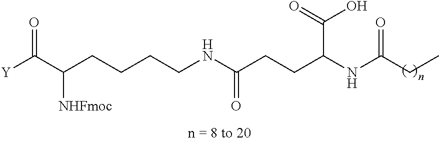 |
| 30 | 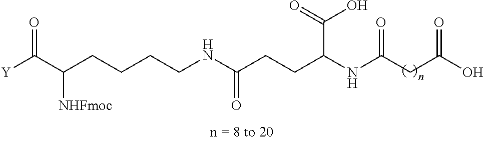 |
| 31 | 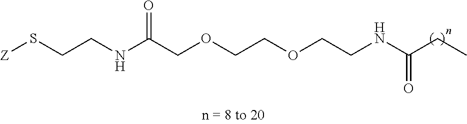 |
| 32 | 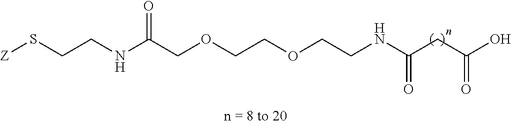 |
| 33 | 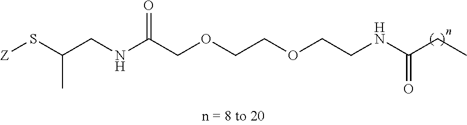 |
| 34 | 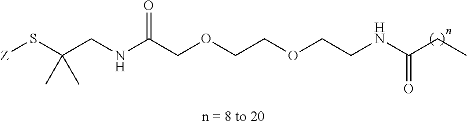 |

*X and Y can be a staple or a staple attached to a therapeutic agent; Z can be S which is part of a staple or part of a staple attached to a therapeutic agent.

TABLE 2
Staple and Staple/HEM

| Staple | Structure |
|---|---|
| L1 | 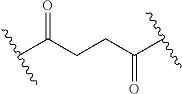 [SMILES: *C(=O)CCC(*)=O] |
| L2 | 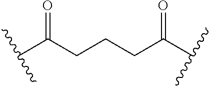 [SMILES: *C(=O)CCCC(*)=O] |
| L3 | 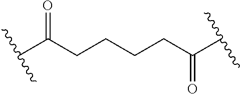 [SMILES: *C(=O)CCCCC(*)=O] |
| L4 | 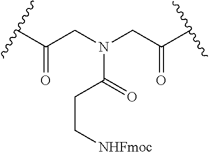 |
| L5 | 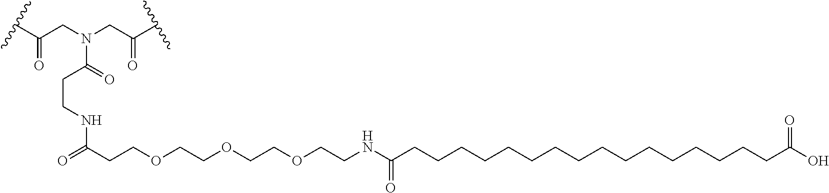 [SMILES: *C(=O)CN(CC(*)=O)C(=O)CCNC(=O)CCOCCOCCOCCNC(=O)CCCCCCCCCCCCCCCCC(=O)O] |
| L6 | 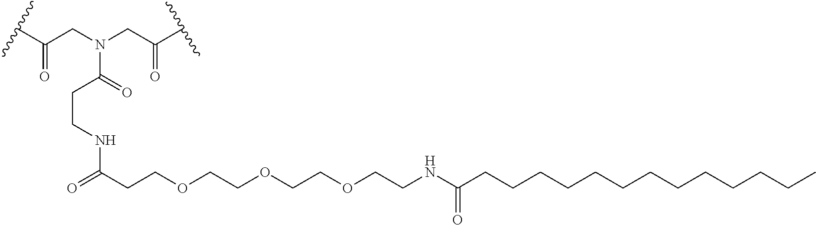 [SMILES: *C(=O)CN(CC(*)=O)C(=O)CCNC(=O)CCOCCOCCOCCNC(=O)CCCCCCCCCCCCC] |
| L7 | 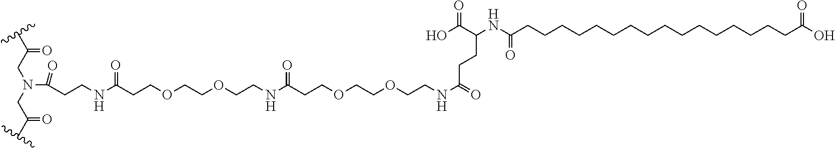 [SMILES: *C(=O)CN(CC(*)=O)C(=O)CCNC(=O)CCOCCOCCNC(=O)CCOCCOCCNC(=O)CCC(NC(=O)CCCCCCCCCCCCCCCCC(=O)O)C(=O)O] |

### Example 4. Generation of CREB Responsive Luciferase Stable Cell Lines Overexpressing Glucagon, GLP-1, GIP, or GLP-2 Receptor

CREB responsive luciferase stable HEK 293 cell line overexpressing human glucagon receptor (GCGR), glucagon-like peptide 1 receptor (GLP-1R), glucose-dependent insulinotropic polypeptide receptor (GIPR), glucagon-like peptide 2 receptor (GLP-2R) was generated as follows.

HEK293 cells were infected with lent virus encoding firefly luciferase gene under the control of CRE promoter, as described in the manual (Qiagen, Netherlands) and then were selected using 1 μg/mL puromycin (Life technologies, Carlsbad) for 1 week. The survived cells were named as CRE-HEK293, expanded and then transfected with a G418 selective mammalian expression plasmid encoding human GCGR, GLP-1R, GIPR or GLP-2R. In brief, GCGR, GLP-1R, GIPR, or GLP-2R plasmid was transfected into CRE-HEK293 cells using Lipofectamine 2000 and selected with 400 μg/mL geneticin (Life technologies, Carlsbad, CA). Single colony stable cell lines overexpressing both CRE-luciferase and GCGR, GLP-1R, GIPR, or GLP-2R were then established for in vitro activity assays. These four stable cell lines were named as HEK293-GCGR-CRE, HEK293-GLP-1R-CRE, HEK293-GIPR-CRE, and HEK293-GLP-2R-CRE.

### Example 5. In Vitro Activity Assays (Receptor-Mediated cAMP Synthesis)

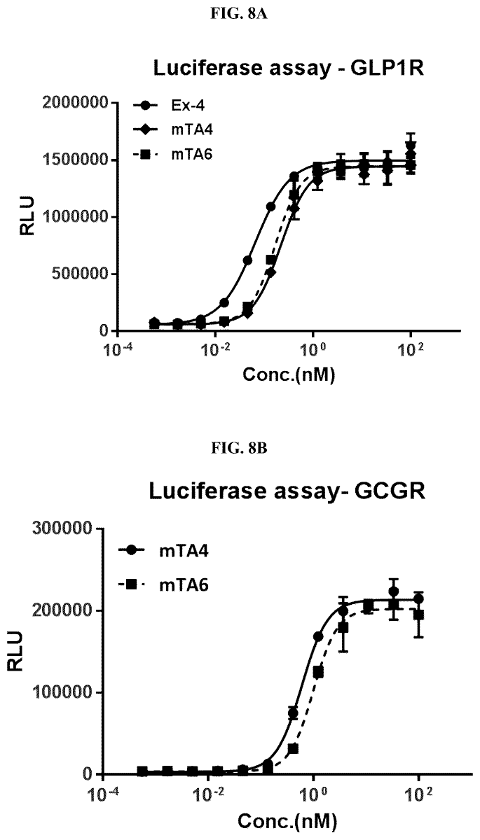

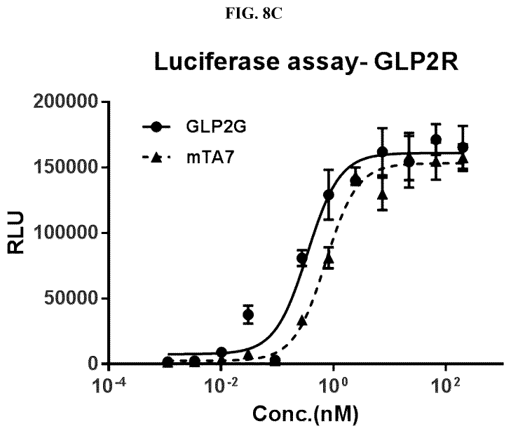

HEK293-GCGR-CRE, HEK293-GLP-1R-CRE, HEK293-GIPR-CRE, and HEK293-GLP-2R-CRE cells were seeded in a white 384-well plate at a density of 5,000 cells per well and cultured for 24 hours. Cells were treated with different peptides in a dose dependent manner. On the next day, 10 μl of Bright-Glo reagent (Promega, Madison, WI) were added into each well and firefly luminescence was determined using an Envision multilabel plate reader (PerkinElmer, Waltham, MA). EC50 of each peptide was calculated using GraphPad Prism 6 software (GraphPad, San Diego, CA). Results are shown in FIG. 8A, FIG. 8B, and FIG. 8C.

### Example 6. In Vivo Pharmacokinetic (PK) Studies

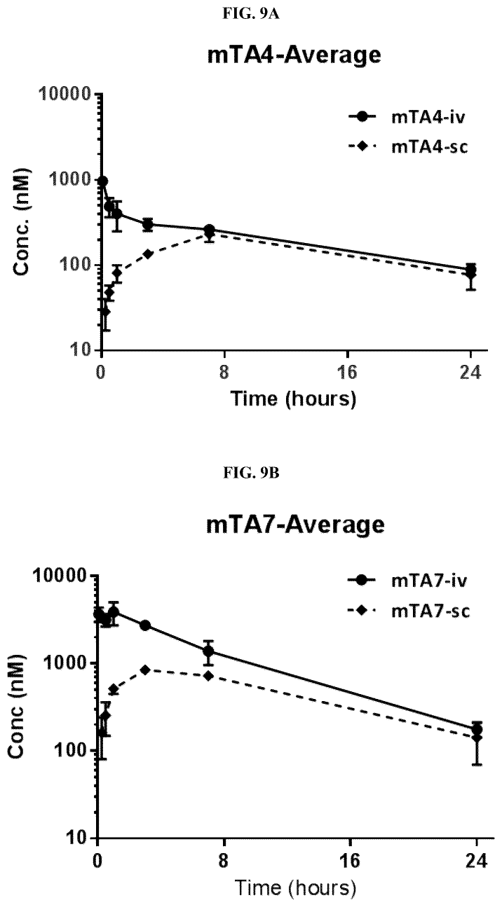

Female CD-1 mice obtained from Charles River Limited were used after overnight food deprivation for in vivo PK study. Peptides were dissolved in pH adjusted Phosphate buffered saline (PBS). 100 μL of each peptide (0.3 mg/kg) was administrated into each mouse through either i.v. or s.c route. Food was provided to mice immediately after bleeding at 30 minute. Blood were extracted into heparinized tubes and centrifuged at 3,000×g for 15 min. The resulting supernatant plasma were then stored at −80° C. for activity assays. The concentrations of peptides in plasma at each time point were determined by activity assay described above and in vivo half-life of each peptide was calculated by using Winnonlin Phoenix software (Pharsight Corp, St. Louis, MO). Results are shown in FIG. 9A and FIG. 9B for mTA4 and mTA7 respectively.

### Example 7. Oral Glucose Tolerance Test (OGTT) and Intraperitoneal Glucose Tolerance Test (IPGTT)

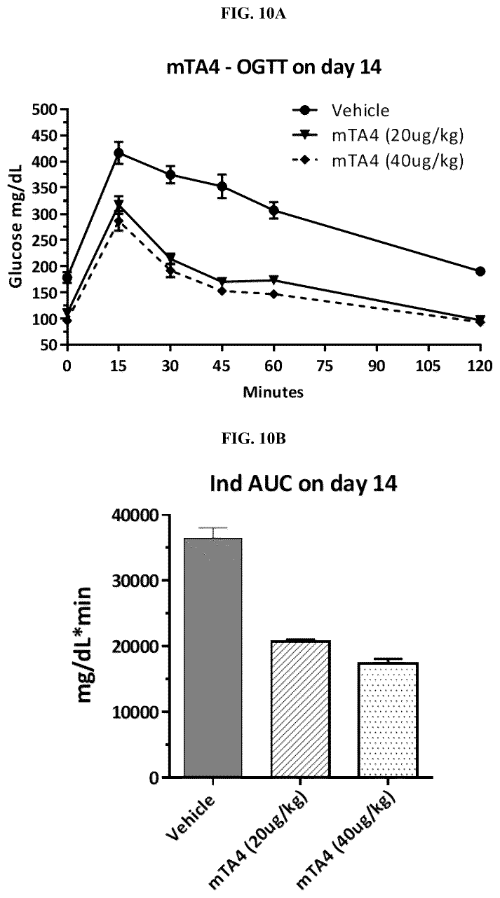

Diet Induced Obese (DIO) mice were fasted overnight and then administered with certain amount of peptides through either i.v. or s.c. route. After 6 hours, mice were orally or intraperitoneally administered with bolus dose of glucose solution at 2 g/kg body mass at concentration of 100 mg/mL and their tail blood glucose levels are measured before (0 min) and after glucose challenge for 2 to 3 hours. Results after a 14 day treatment with mTA4 (s.c.) are shown in FIG. 10A and FIG. 10B.

### Example 8. Body Weight, Food Intake, and Visceral Fat Mass Measurement

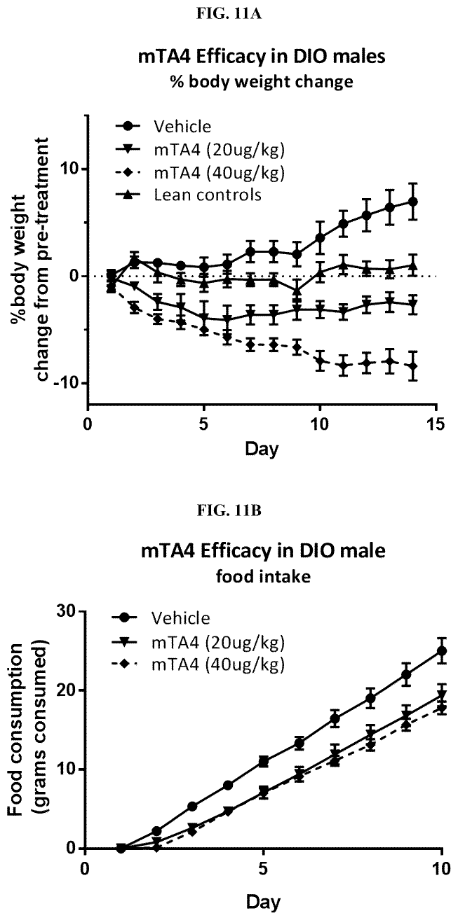

Diet Induced Obese (DIO) mice were purchased from Charles River and a GLP-1R agonist or a GLP-1R/GCGR dual agonist was administered by s.c. injection. The mouse body weight and food intake were monitored daily for 2 weeks, and followed before (5 days in total) and during treatment (5-weeks in total). After 5 weeks, mice were sacrificed and visceral fat mass were taken out and weighed. Results on body weight and food intake after a 2 week treatment are shown in FIG. 11A and FIG. 11B respectively.

### Example 9. Cholesterol Level Determination

Collected plasma is used for cholesterol level determination according to the manufacturer's guide (cholesterol assay kit, Abcam, Cambridge, England). Briefly, plasma is diluted using cholesterol assay buffer and then reacted with same volume of reaction mix containing cholesterol assay buffer, cholesterol probe, enzyme mix and cholesterol esterase. After incubation at 37° C. for 1 hour, the absorbance at 560 nm is measured using an Envision multilabel plate reader (PerkinElmer, Waltham, MA). Subsequently, the concentration of cholesterol in plasma is calculated using a standard curve.

### Example 10. Triglyceride Level Measurement

Collected plasma is again used for triglyceride level determination using a triglyceride colorimetric assay kit (Cayman chemical, Ann Arbor, Michigan). 5 μL of plasma samples or standard are added into a 384 well plate and followed by adding 75 μL of diluted enzyme buffer to each well. The plate is incubated at room temperature for 15 min, and the absorbance is read at 560 nm using an Envision multilabel plate reader (PerkinElmer, Waltham, MA). Again, the concentration of triglyceride in plasma is calculated using a standard curve.

### Example 11. Oil Red O Staining (Lipid Droplet Staining)

Frozen tissue sections of liver are cut at 10 μm and air dried to the slides. After fixation in 10% formalin for 5 min, the slides are briefly washed with running tap water for 10 min, followed by rinse with 60% isopropanol. Subsequently, oil red O working solution (0.3% oil red O) is used for lipid staining for 15 min. Slides are again rinsed with 60% isopropanol and then nuclei are lightly stained with alum haematoxylin, followed by rinse with distilled water and mounted in glycerine jelly. After half an hour, pictures are taken under microscopy.

### Example 12. Application of Micro-Needle Based Patch on Guinea Pigs

Microneedle patch based transdermal drug delivery is performed using guinea pigs. Guinea pigs are shaved one day before patch application. On the second day, micro-needle based patches are applied onto the animal skin for 5 min and blood is extracted at different time points (5 min, 30 min, 1 h, 2 h, 3 h, 5 h, 8 h, 24 h, 32 h, 48 h, 72 h). Peptide concentration at each time point is determined using the same method (functional activity assay) as described above. Transdermal bioavailability (F) is calculated as the ratio of area under the curve (AUC) between microneedle patch application and i.v. injection groups.

### Example 13. Acute Murine Model of Ulcerative Colitis (Acute DSS Model)

Colitis in mouse was induced by adding 3% DSS (dextran sulfate sodium) in the drinking water for 5 consecutive days. Besides DSS placement, mice were treated twice daily with GLP-2 analogs, cyclosporine A (20 mg/kg/day) was used for the positive control group and PBS for the negative control group. During the experiment period, body weight was measured every day.

### Example 14. Chronic Murine Model of Ulcerative Colitis (Chronic DSS Model)

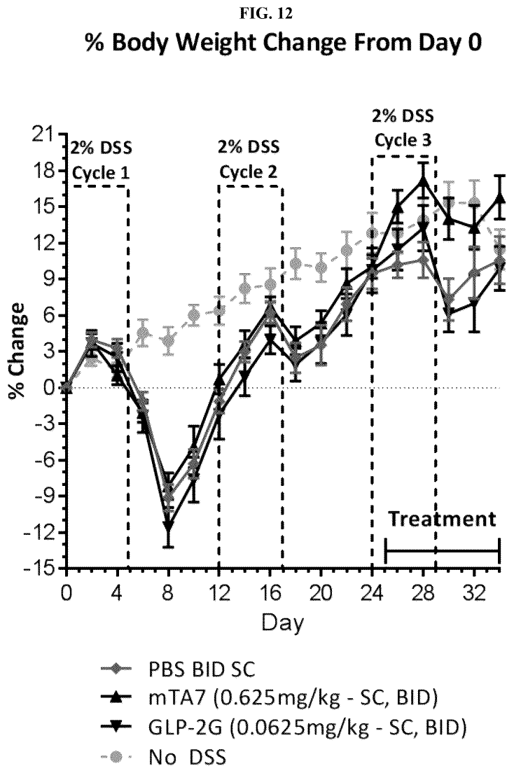

Colitis in 9 weeks old B6 male mice (n=12/group) was induced by adding 2% DSS (dextran sulfate sodium) in the drinking water for 5 consecutive days and then changed into normal drinking water for 7 days, followed by two more cycles of DSS (5 days on, 7 days off) in drinking water ad libitum, body weight monitoring every the other day. At the beginning of third cycle of DSS treatment, mice were treated twice daily with GLP-2 and mTA7, cyclosporine A (20 mg/kg/day) is used for the positive control group and PBS for the negative control group. Results are shown in FIG. 12.

### Example 15. Intestine/Colon Weight/Length Measurement

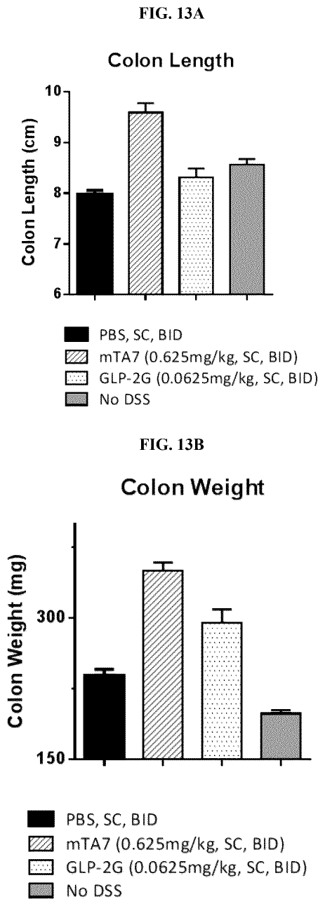

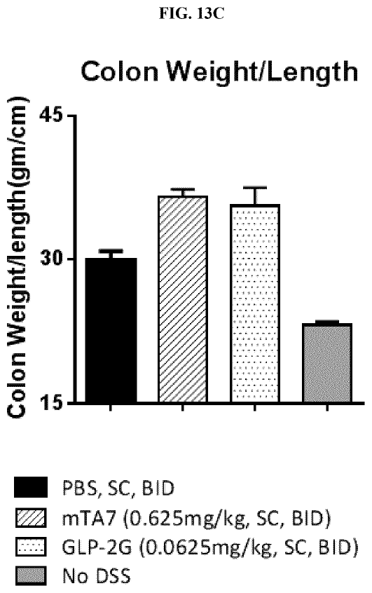

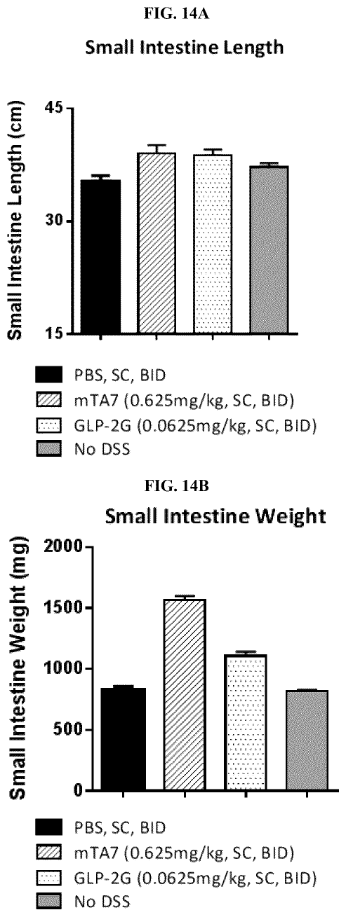

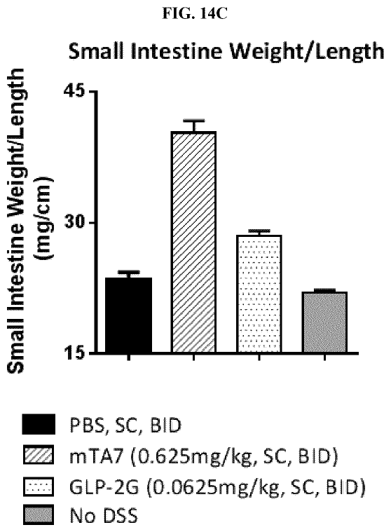

After the treatment, animals were euthanized by CO₂ inhalation, a midline incision was made to provide access to the gastrointestinal tract. The small intestine length was determined as the distance from the pylorus to the cecum. Similarly, the colonic length was recorded as distance from the ileocecal junction to the rectum. Fetal material was removed from both small intestine and colonic segments and their respective wet weights recorded. After measurement of the colon weight and length, large colon were divided into 3 fragments (proximal, middle and distal), fixed half of each fragment individually and sent out for histology, flesh freezed another half using dry ice and keep at −80 degree for qPCR and other assays. Results are shown in FIG. 13A-13C and FIG. 14A-14C.

### Example 16. Efficacy and Safety of mTA for the Treatment of Diabetes and Obesity

Purpose: Different doses of mTA are compared to placebo to determine efficacy and safety for the treatment of patients with diabetes and obesity.

| Condition | Intervention | Phase |
|---|---|---|
| Diabetes and | Drug: single agonist or dual agonist mTA | Phase 1 |
| Obesity | Drug: Placebo | Phase 1 |

Study Type: Interventional
Study Design: Allocation: Randomized
Endpoint Classification: Safety/Efficacy Study
Intervention Model: Parallel Assignment
Masking: Double Blind (Subject, Caregiver, Investigator, Outcomes Assessor)
Primary Purpose: Treatment
Primary Outcome Measures: reduced blood glucose, reduced body weight and HbA1C [Time Frame: Up to 52 weeks][Designated as safety issue: No]
Secondary Outcome Measures: improved metabolic profiles
[Time Frame: Up to day 52 weeks][Designated as safety issue: No]

| Arms | Assigned Intervention |
|---|---|
| Placebo Comparator: Placebo | Drug: Placebo |
| subcutaneous or microneedle | subcutaneous or microneedle |
| patch delivery | patch delivery |
| Experimental: single and dual | Drug: single and dual agonist |
| agonist mTA through | mTA through subcutaneous or |
| subcutaneous or microneedle | microneedle patch delivery |
| patch delivery | |

This is an international, randomized, double-blind, placebo-controlled, Phase II/III trial of single and dual agonist mTA for the treatment of diabetes and obesity.

TABLE 3
Therapeutic Agents (TAs)- Amino acid sequences

| | SEQ ID | |
|---|---|---|
| NAME | NO | SEQUENCE |
| Oxyntomodulin | 1 | HSQGTFTSDYSKYLDSRRAQDFVQWLMNTKRNRNNIA |
| Exendin-4 | 2 | HGEGTFTSDLSKQMEEEAVRLFIEWLKNGGPSSGAPPPS |
| hGLP-1 | 3 | HAEGTFTSDVSSYLEGQAAKEFIAWLVKGR |
| Glucagon | 4 | HSQGTFTSDYSKYLDSRRAQDFVQWLMNT |
| hGLP-2 | 5 | HADGSFSDEMNTILDNLAARDFINWLIQTKITD |
| hGIP | 6 | YAEGTFISDYSIAMDKIHQQDFVNWLLAQKGKKNDWKH |
| | | NITQ |

TABLE 4
Therapeutic Agents: Modified Therapeutic Peptides

| | SEQ ID | |
|---|---|---|
| NAME | NO | SEQUENCE^{a,b,c} |
| Exendin-4 (A) | 7 | HGEGTFTSDLSKQMEEXAVRLFIXWLKNGGPSSGAPPPS |
| Exendin-4 (B) | 8 | HGEGTFTSDLSKXMEEEAVRLFIXWLKNGGPSSGAPPPS |
| Exendin-4 (C) | 9 | HGEGTFTSDXSKQMEEEAVRLFIXWLKNGGPSSGAPPPS |
| OXM (A) | 10 | HsQGTFTSDYSKYLDSXRAQDFVXWLMNTKRNRNNIA |
| OXM (B) | 11 | HsQGTFTSDYSKYLDEXAAKEFIXWLMNTKRNRNNIA |
| GLP-1/Glucagon | 12 | HsQGTFTSDYSKYLDSXAAHDFVXWLLRA |
| (A) | | |
| GLP-1/Glucagon | 13 | HsQGTFTSDYSKYLDEXAAKEFIXWLLRA |
| (B) | | |
| GLP-1/Glucagon | 14 | HsQGTFTSDYSKYLDEXAAKEFIXWLLRAGPSSGAPPPS |
| (C) | | |
| GLP-1/Glucagon | 15 | HsQGTFTSDYSKYLDEXAAKEFIXWLLNGGPSSGAPPPS |
| (D) | | |
| GLP-1/GIP (A) | 16 | YaEGTFTSDYSIYLDKXAAKEFVXWLLAGGPSSGAPPPS |
| GLP-1/GIP/ | 17 | YaEGTFISDYSKYLDEXAAKEFIXWLMNTKRNRNNIA |
| Glucagon (A) | | |
| GLP-1/GIP/ | 18 | H-Aib-QGTFTSDKSKYLDEXAAQDFVXWLLDGGPSSGAPPPS |
| Glucagon (B) | | |
| GLP-2 (A) | 19 | HGDGSFSDEMNTILDNXAARDFIXWLIQTKITD |
| GLP-2 (B) | 20 | HGDGSFSDELLTILDLXAARDFIXWLIQTKITD |
| GLP-2 (C) | 21 | HGDGSFSDEMNTILDXLAARDFIXWLIQTKITD |
| GLP-2 (D) | 22 | HGDGSFSXEMNTILXALAARDFINWLIQTKITD |
| GLP-2 (E) | 23 | HGDGSFSDEMNTILDALAARXFINWLIXTKITD |
| GLP-2 (F) | 24 | HGDGSFSDXMNTILDXLAARDFINWLIQTKITD |
| GLP-2 (G) | 25 | HGDGSFSDEMXTILDNLXARDFINWLIQTKITD |
| GLP-2 (H) | 26 | HGDGSFSDEMNTILDNXAARDFIXWLIQTKITDPSSGAPPPS |
| GLP-2 (I) | 27 | HGDGSFSDXMNTILDXLAARDFINWLIQTKITDPSSGAPPPS |
| GLP-2 (J) | 28 | HGDGSFSDEMXTILDNLXARDFINWLIQTKITDPSSGAPPPS |
| GLP-1 (A) | 29 | HGEGTFTSDVSSYLEGXAAKEFIXWLVKGR |
| GLP-1 (B) | 30 | H-Aib-EGTFTSDVSSYLEGXAAKEFIXWLVKGR |

^{a}X is an amino acid with an amine-containing sidechain (e.g., lysine, ornithine, diaminobutyric acid, diaminopropionic acid, or homolysine).
^{b}Lower case represent D-amino acids.
^{c}Aib = 2-Aminoisobutyric acid.

TABLE 5
Modified Therapeutic Agents

| | | Mass | Mass | GLP-1R | GCGR | t_{1/2} | |
|---|---|---|---|---|---|---|---|
| mTA | Structure^{a} | Expected | Found | (EC₅₀, nM) | (EC₅₀, nM) | (h) | |
| 1 | Exendin-4 (A) and L1 | 4266.8 | 1423.6 | 0.03 | ND | ND | |
| | | | ([M + 3H]³⁺), | | | | |
| | | | 1068.0 | | | | |
| | | | ([M + 4H]⁴⁺) | | | | |
| 2 | Exendin-4 (A) and L2 | 4280.8 | 1427.6 | 0.02 | ND | ND | |
| | | | ([M + 3H]³⁺), | | | | |
| | | | 1071.0 | | | | |
| | | | ([M + 4H]⁴⁺) | | | | |
| 3 | Exendin-4 (A) and L3 | 4294.8 | 1432.5 | 0.03 | ND | ND | |
| | | | ([M + 3H]³⁺), | | | | |
| | | | 1074.3 | | | | |
| | | | ([M + 4H]⁴⁺) | | | | |
| 4 | GLP-1/Glucagon (B) | 4100.7 | 1444.7 | 0.03 | 0.15 | T_{1/2} = | 5.7 h (iv); |
| | and L5 | | ([M + 3H]³⁺), | | | Tₘₐₓ = | 8 h (sc) |

| | | | | | | |
|---|---|---|---|---|---|---|
| | | | 1083.8 | | | |
| | | | ([M + 4H]⁴⁺) | | | |
| 5 | GLP-1/Glucagon (D) | 4307.7 | 1436.6 | 0.02 | 0.07 | ND |
| | and L2 | | ([M + 3H]³⁺), | | | |
| | | | 1077.7 | | | |
| | | | ([M + 4H]⁴⁺) | | | |
| 6 | GLP-1/Glucagon (D) | 4923.5 | 1641.9 | 0.03 | 0.62 | ND |
| | and L5 | | ([M + 3H]³⁺), | | | |
| | | | 1231.7 | | | |
| | | | ([M + 4H]⁴⁺) | | | |

^{a}Structure is described with X = lysine.

TABLE 6
Modified Therapeutic Agents

| | | | | GLP-2R | |
|---|---|---|---|---|---|
| | | Mass | | (EC₅₀, | |
| mTA | Structure^{a} | Expected | Mass Found | nM) | t_{1/2} (h) |
| 7 | GLP-2 | 4404.1 | 1641.9 | 0.068 | T_{1/2} = 3 h (iv); |
| | (G) and | | ([M + 3H]³⁺), | | Tₘₐₓ = 4 h (sc) |
| | L6 | | 1231.7 | | |
| | | | ([M + 4H]⁴⁺) | | |

^{a}Structure is described with X = lysine.

TABLE 7
Modified Therapeutic Agents

| mTA | Structure^{a} | Mass Expected | Mass Found |
|---|---|---|---|
| 9 | GLP-2 (G) and L5 | 4490.1 | ND |
| 12 | GLP-2 (A) and L6 | 4361.9 | ND |
| 13 | GLP-2 (A) and L5 | 4448.1 | ND |
| 14 | OXM (B) and L6 | 4987.9 | ND |
| 15 | GLP-1/GIP (A) | 4731.6 | ND |
| | and L6 | | |
| 16 | GLP-1 (A) and L5 | 4008.8 | ND |

^{a}Structure is described with X = lysine.

1-41. (canceled)

42. A modified therapeutic agent (mTA) of Formula (I): 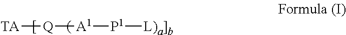
wherein:
TA is a therapeutic agent, wherein the therapeutic agent is a single peptide comprising an amino acid sequence that is at least 95% homologous to any one of SEQ ID NO: 7 and 12-30;
Q is 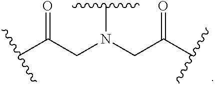 [SMILES: *C(=O)CN(*)CC(*)=O]
A¹ is 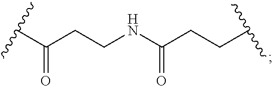 [SMILES: *CCC(=O)NCCC(*)=O]
P¹ is PEG-A²; wherein:
PEG is 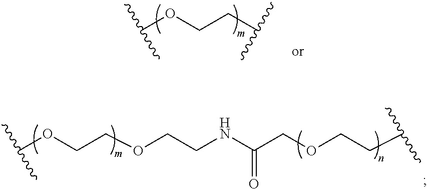
m and n are each 1, 2, 3, or 4; and
A² is 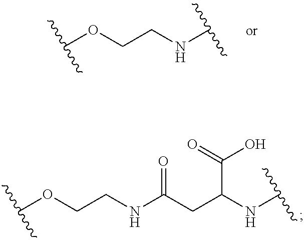 [SMILES: *NCCO* ; *NC(CC(=O)NCCO*)C(=O)O]
L is a lipid derivative selected from a group consisting of sterols, bile acids, vitamin E, fatty di-acids, fatty acids, fatty amides, fatty amines, and fatty alcohols;
a is 1; and
b is 1;
wherein the TA is covalently attached to Q via two amine-containing sidechain amino acids on the single peptide; and wherein the two amine-containing sidechain amino acids are at least two amino acids apart, and less than 20 amino acids apart.

43. The mTA of claim 42, wherein the two amine-containing sidechain amino acids are 6 amino acids apart.

44. The mTA of claim 42, wherein each of the two amine-containing sidechain amino acids is lysine.

45. The mTA of claim 42, wherein-Q-A¹-P¹-L is:

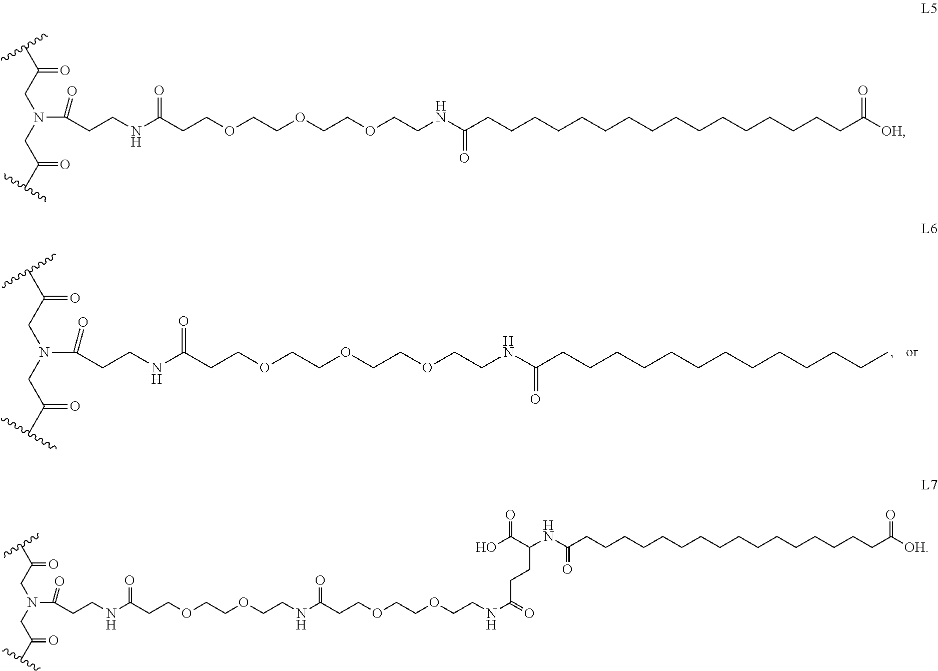 [SMILES: *C(=O)CN(CC(*)=O)C(=O)CCNC(=O)CCOCCOCCOCCNC(=O)CCCCCCCCCCCCCCCCC(=O)O ; *C(=O)CN(CC(*)=O)C(=O)CCNC(=O)CCOCCOCCOCCNC(=O)CCCCCCCCCCCCC ; *C(=O)CN(CC(*)=O)C(=O)CCNC(=O)CCOCCOCCNC(=O)CCOCCOCCNC(=O)CCC(NC(=O)CCCCCCCCCCCCCCCCC(=O)O)C(=O)O]

46. The mTA of claim 42, wherein the single peptide is an exendin-4 agonist, GLP-1R agonist, GLP-2R agonist, GLP-1R/GCGR dual agonist, GLP-1R/GIPR dual agonist, or GLP-1R/GCGR/GIPR tri-agonist.

47. The mTA of claim 42, wherein the single peptide is an exendin-4 agonist.

48. The mTA of claim 42, wherein the single peptide has an amino acid sequence that is at least 95% homologous to SEQ ID NO: 7.

49. The mTA of claim 42, wherein the single peptide is an GLP-1R/GCGR dual agonist.

50. The mTA of claim 42, wherein the single peptide has an amino acid sequence that is at least 95% homologous to SEQ ID NO: 15.

51. The mTA of claim 42, wherein the single peptide is a GLP-2R agonist.

52. The mTA of claim 42, wherein the single peptide has an amino acid sequence that is at least 95% homologous to SEQ ID NO: 26.

53. A pharmaceutical composition comprising the mTA of claim 42, and a pharmaceutically acceptable excipient.

54. A method for treating a disease or condition in a subject in need thereof, the method comprising administering to the subject a composition comprising a therapeutically effective amount of the mTA of claim 42, wherein the disease or condition is diabetes, obesity, or a medical condition associated with diabetes or obesity,

55. A method for treating a disease or condition in a subject in need thereof, the method comprising administering to the subject a composition comprising a therapeutically effective amount of the mTA of claim 42, wherein the disease or condition is cardiovascular disease, psoriasis, non-alcoholic fatty liver disease, nonalcoholic steatohepatitis, ulcerative colitis, Crohn's disease, inflammatory bowel disease, inflammatory bowel syndrome, or short bowel syndrome.

56. A modified therapeutic agent (mTA) comprising (i) a therapeutic agent, (ii) a half-life extending molecule, and (iii) a staple, wherein:
(i) the therapeutic agent is a single peptide chain having an amino acid sequence that is at least 95% homologousto any one of SEQ ID NOs: 7 and 12-30 that is covalently attached to the staple via two amine-containing sidechain amino acids on the single peptide chain; and
wherein the two amine-containing sidechain amino acids are at least two amino acids apart, and less than 20 amino acids apart;
(ii) the half-life extending molecule is covalently attached to the first staple; and
(iii) the staple has the following structure:

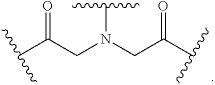 [SMILES: *C(=O)CN(*)CC(*)=O]

57. The mTA of claim 56, wherein the staple and the half-life extending molecule have a structure according to L⁴, L⁵, L⁶, or L⁷:

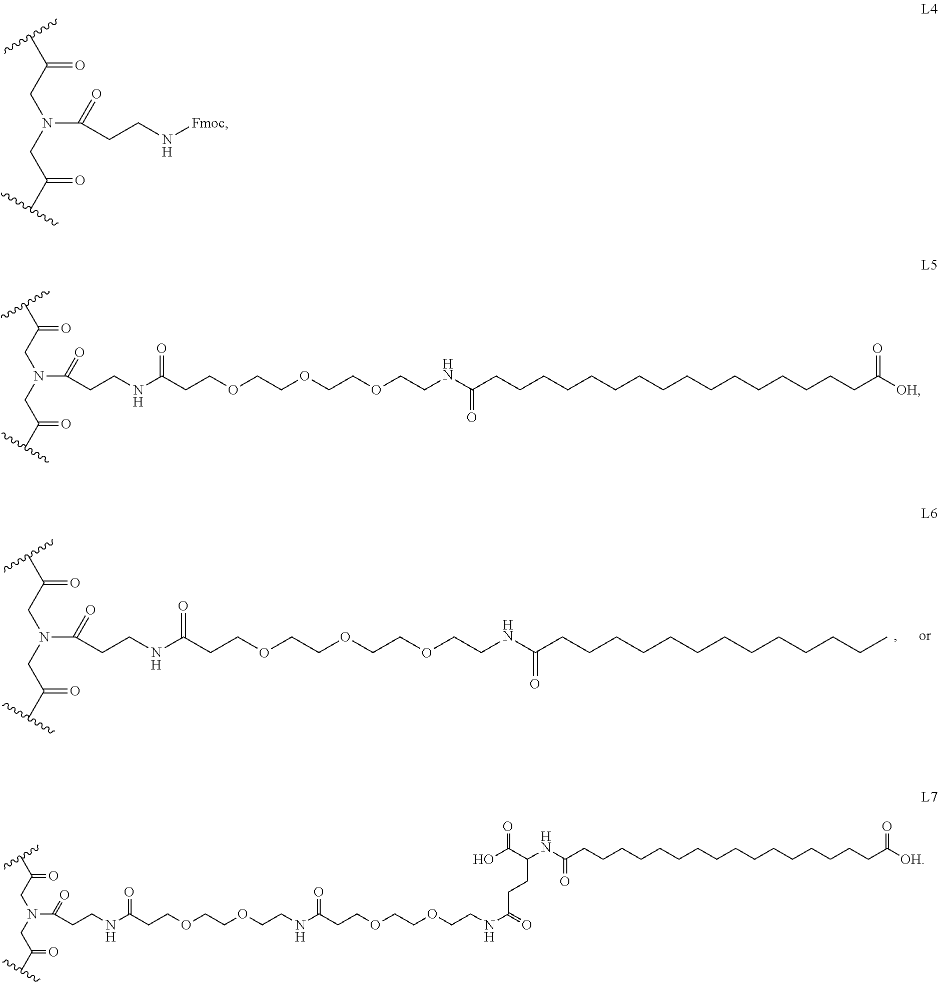

58. A modified therapeutic agent (mTA) comprising (i) a therapeutic agent, (ii) a half-life extending molecule, and (iii) a staple, wherein:
(i) the therapeutic agent is a single peptide GLP-1R/GCGR/GIPR tri-agonist that is covalently attached to the staple via two amine-containing sidechain amino acids on the single peptide; wherein the two amine-containing sidechain amino acids are at least two amino acids apart, and less than 20 amino acids apart;
(ii) the half-life extending molecule is covalently attached to the first staple; and
(iii) the staple has the following structure:

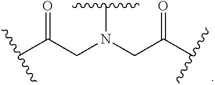 [SMILES: *C(=O)CN(*)CC(*)=O]

59. The mTA of claim 58, wherein the half-life extending molecule comprises a lipid, a polyglycol, a peptide, or a combination thereof.

60. The mTA of claim 59, wherein the lipid is a fatty diacid, fatty acid, fatty amide, fatty amine, or fatty alcohol having 10 to 40 carbon atoms.

61. The mTA of claim 59, wherein the lipid is selected from dodecanoic acid, dodecanedioic acid, tridecanoic acid, tridecanedioic acid, tetradecanoic acid, tetradecanedioic acid, myristic acid, pentadecanoic acid, pentadecanedioic acid, hexadecanoic acid, hexadecanedioic acid, heptadecanoic acid, heptadecanedioic acid, octadecanoic acid, octadecanedioic acid, nonadecanoic acid, nonadecanedioic acid, eicosanoic acid, or eicosanedioic acid.


SEQUENCE LISTING (33 sequences)

SEQ ID NO: 1 - protein, 37 aa, unidentified
  REGION 1..37: note = Description of Unknown: Oxyntomodulin polypeptide
HSQGTFTSDYSKYLDSRRAQDFVQWLMNTKRNRNNIA

SEQ ID NO: 2 - protein, 39 aa, unidentified
  REGION 1..39: note = Description of Unknown: Exendin-4 polypeptide
HGEGTFTSDLSKQMEEEAVRLFIEWLKNGGPSSGAPPPS

SEQ ID NO: 3 - protein, 30 aa, unidentified
  REGION 1..30: note = Description of Unknown: hGLP-1 polypeptide
HAEGTFTSDVSSYLEGQAAKEFIAWLVKGR

SEQ ID NO: 4 - protein, 29 aa, unidentified
  REGION 1..29: note = Description of Unknown: Glucagon peptide
HSQGTFTSDYSKYLDSRRAQDFVQWLMNT

SEQ ID NO: 5 - protein, 33 aa, unidentified
  REGION 1..33: note = Description of Unknown: hGLP-2 polypeptide
HADGSFSDEMNTILDNLAARDFINWLIQTKITD

SEQ ID NO: 6 - protein, 42 aa, unidentified
  REGION 1..42: note = Description of Unknown: hGIP polypeptide
YAEGTFISDYSIAMDKIHQQDFVNWLLAQKGKKNDWKHNITQ

SEQ ID NO: 7 - protein, 39 aa, synthetic construct
  REGION 1..39: note = Description of Artificial Sequence: Synthetic polypeptide
  VARIANT 17: note = Any amino acid with an amine-containing side chain
  VARIANT 24
HGEGTFTSDLSKQMEEXAVRLFIXWLKNGGPSSGAPPPS

SEQ ID NO: 8 - protein, 39 aa, synthetic construct
  REGION 1..39: note = Description of Artificial Sequence: Synthetic polypeptide
  VARIANT 13: note = Any amino acid with an amine-containing side chain
  VARIANT 24: note = Any amino acid with an amine-containing side chain
HGEGTFTSDLSKXMEEEAVRLFIXWLKNGGPSSGAPPPS

SEQ ID NO: 9 - protein, 39 aa, synthetic construct
  REGION 1..39: note = Description of Artificial Sequence: Synthetic polypeptide
  VARIANT 10: note = Any amino acid with an amine-containing side chain
  VARIANT 24: note = Any amino acid with an amine-containing side chain
HGEGTFTSDXSKQMEEEAVRLFIXWLKNGGPSSGAPPPS

SEQ ID NO: 10 - protein, 37 aa, synthetic construct
  REGION 1..37: note = Description of Artificial Sequence: Synthetic polypeptide
  MOD_RES 2: note = D-Ser
  VARIANT 17: note = Any amino acid with an amine-containing side chain
  VARIANT 24: note = Any amino acid with an amine-containing side chain
HSQGTFTSDYSKYLDSXRAQDFVXWLMNTKRNRNNIA

SEQ ID NO: 11 - protein, 37 aa, synthetic construct
  REGION 1..37: note = Description of Artificial Sequence: Synthetic polypeptide
  MOD_RES 2: note = D-Ser
  VARIANT 17: note = Any amino acid with an amine-containing side chain
  VARIANT 24: note = Any amino acid with an amine-containing side chain
HSQGTFTSDYSKYLDEXAAKEFIXWLMNTKRNRNNIA

SEQ ID NO: 12 - protein, 29 aa, synthetic construct
  REGION 1..29: note = Description of Artificial Sequence: Synthetic peptide
  MOD_RES 2: note = D-Ser
  VARIANT 17: note = Any amino acid with an amine-containing side chain
  VARIANT 24: note = Any amino acid with an amine-containing side chain
HSQGTFTSDYSKYLDSXAAHDFVXWLLRA

SEQ ID NO: 13 - protein, 29 aa, synthetic construct
  REGION 1..29: note = Description of Artificial Sequence: Synthetic peptide
  MOD_RES 2: note = D-Ser
  VARIANT 17: note = Any amino acid with an amine-containing side chain
  VARIANT 24: note = Any amino acid with an amine-containing side chain
HSQGTFTSDYSKYLDEXAAKEFIXWLLRA

SEQ ID NO: 14 - protein, 39 aa, synthetic construct
  REGION 1..39: note = Description of Artificial Sequence: Synthetic polypeptide
  MOD_RES 2: note = D-Ser
  VARIANT 17: note = Any amino acid with an amine-containing side chain
  VARIANT 24: note = Any amino acid with an amine-containing side chain
HSQGTFTSDYSKYLDEXAAKEFIXWLLRAGPSSGAPPPS

SEQ ID NO: 15 - protein, 39 aa, synthetic construct
  REGION 1..39: note = Description of Artificial Sequence: Synthetic polypeptide
  MOD_RES 2: note = D-Ser
  VARIANT 17: note = Any amino acid with an amine-containing side chain
  VARIANT 24: note = Any amino acid with an amine-containing side chain
HSQGTFTSDYSKYLDEXAAKEFIXWLLNGGPSSGAPPPS

SEQ ID NO: 16 - protein, 39 aa, synthetic construct
  REGION 1..39: note = Description of Artificial Sequence: Synthetic polypeptide
  MOD_RES 2: note = D-Ala
  VARIANT 17: note = Any amino acid with an amine-containing side chain
  VARIANT 24: note = Any amino acid with an amine-containing side chain
YAEGTFTSDYSIYLDKXAAKEFVXWLLAGGPSSGAPPPS

SEQ ID NO: 17 - protein, 37 aa, synthetic construct
  REGION 1..37: note = Description of Artificial Sequence: Synthetic polypeptide
  MOD_RES 2: note = D-Ala
  VARIANT 17: note = Any amino acid with an amine-containing side chain
  VARIANT 24: note = Any amino acid with an amine-containing side chain
YAEGTFISDYSKYLDEXAAKEFIXWLMNTKRNRNNIA

SEQ ID NO: 18 - protein, 39 aa, synthetic construct
  REGION 1..39: note = Description of Artificial Sequence: Synthetic polypeptide
  MOD_RES 2: note = Aib
  VARIANT 17: note = Any amino acid with an amine-containing side chain
  VARIANT 24: note = Any amino acid with an amine-containing side chain
HXQGTFTSDKSKYLDEXAAQDFVXWLLDGGPSSGAPPPS

SEQ ID NO: 19 - protein, 33 aa, synthetic construct
  REGION 1..33: note = Description of Artificial Sequence: Synthetic polypeptide
  VARIANT 17: note = Any amino acid with an amine-containing side chain
  VARIANT 24: note = Any amino acid with an amine-containing side chain
HGDGSFSDEMNTILDNXAARDFIXWLIQTKITD

SEQ ID NO: 20 - protein, 33 aa, synthetic construct
  REGION 1..33: note = Description of Artificial Sequence: Synthetic polypeptide
  VARIANT 17: note = Any amino acid with an amine-containing side chain
  VARIANT 24: note = Any amino acid with an amine-containing side chain
HGDGSFSDELLTILDLXAARDFIXWLIQTKITD

SEQ ID NO: 21 - protein, 33 aa, synthetic construct
  REGION 1..33: note = Description of Artificial Sequence: Synthetic polypeptide
  VARIANT 16: note = Any amino acid with an amine-containing side chain
  VARIANT 24: note = Any amino acid with an amine-containing side chain
HGDGSFSDEMNTILDXLAARDFIXWLIQTKITD

SEQ ID NO: 22 - protein, 33 aa, synthetic construct
  REGION 1..33: note = Description of Artificial Sequence: Synthetic polypeptide
  VARIANT 8: note = Any amino acid with an amine-containing side chain
  VARIANT 15: note = Any amino acid with an amine-containing side chain
HGDGSFSXEMNTILXALAARDFINWLIQTKITD

SEQ ID NO: 23 - protein, 33 aa, synthetic construct
  REGION 1..33: note = Description of Artificial Sequence: Synthetic polypeptide
  VARIANT 21: note = Any amino acid with an amine-containing side chain
  VARIANT 28: note = Any amino acid with an amine-containing side chain
HGDGSFSDEMNTILDALAARXFINWLIXTKITD

SEQ ID NO: 24 - protein, 33 aa, synthetic construct
  REGION 1..33: note = Description of Artificial Sequence: Synthetic polypeptide
  VARIANT 9: note = Any amino acid with an amine-containing side chain
  VARIANT 16: note = Any amino acid with an amine-containing side chain
HGDGSFSDXMNTILDXLAARDFINWLIQTKITD

SEQ ID NO: 25 - protein, 33 aa, synthetic construct
  REGION 1..33: note = Description of Artificial Sequence: Synthetic polypeptide
  VARIANT 11: note = Any amino acid with an amine-containing side chain
  VARIANT 18: note = Any amino acid with an amine-containing side chain
HGDGSFSDEMXTILDNLXARDFINWLIQTKITD

SEQ ID NO: 26 - protein, 42 aa, synthetic construct
  REGION 1..42: note = Description of Artificial Sequence: Synthetic polypeptide
  VARIANT 17: note = Any amino acid with an amine-containing side chain
  VARIANT 24: note = Any amino acid with an amine-containing side chain
HGDGSFSDEMNTILDNXAARDFIXWLIQTKITDPSSGAPPPS

SEQ ID NO: 27 - protein, 42 aa, synthetic construct
  REGION 1..42: note = Description of Artificial Sequence: Synthetic polypeptide
  VARIANT 9: note = Any amino acid with an amine-containing side chain
  VARIANT 16: note = Any amino acid with an amine-containing side chain
HGDGSFSDXMNTILDXLAARDFINWLIQTKITDPSSGAPPPS

SEQ ID NO: 28 - protein, 42 aa, synthetic construct
  REGION 1..42: note = Description of Artificial Sequence: Synthetic polypeptide
  VARIANT 11: note = Any amino acid with an amine-containing side chain
  VARIANT 18: note = Any amino acid with an amine-containing side chain
HGDGSFSDEMXTILDNLXARDFINWLIQTKITDPSSGAPPPS

SEQ ID NO: 29 - protein, 30 aa, synthetic construct
  REGION 1..30: note = Description of Artificial Sequence: Synthetic polypeptide
  VARIANT 17: note = Any amino acid with an amine-containing side chain
  VARIANT 24: note = Any amino acid with an amine-containing side chain
HGEGTFTSDVSSYLEGXAAKEFIXWLVKGR

SEQ ID NO: 30 - protein, 30 aa, synthetic construct
  REGION 1..30: note = Description of Artificial Sequence: Synthetic polypeptide
  MOD_RES 2: note = Aib
  VARIANT 17: note = Any amino acid with an amine-containing side chain
  VARIANT 24: note = Any amino acid with an amine-containing side chain
HXEGTFTSDVSSYLEGXAAKEFIXWLVKGR

SEQ ID NO: 31 - protein, 29 aa, synthetic construct
  REGION 1..29: note = Description of Artificial Sequence: Synthetic peptide
  MOD_RES 2: note = D-Ser
HSQGTFTSDYSKYLDEKAAKEFIKWLLRA

SEQ ID NO: 32 - protein, 39 aa, synthetic construct
  REGION 1..39: note = Description of Artificial Sequence: Synthetic polypeptide
  MOD_RES 2: note = D-Ser
HSQGTFTSDYSKYLDEKAAKEFIKWLLNGGPSSGAPPPS

SEQ ID NO: 33 - protein, 33 aa, synthetic construct
  REGION 1..33: note = Description of Artificial Sequence: Synthetic polypeptide
HGDGSFSDEMKTILDNLKARDFINWLIQTKITD